(19) United States
(12) Patent Application Publication
(10) Pub. No.: US 2025/0161025 A1
(43) Pub. Date: May 22, 2025

(54) COMPOSITIONS, DEVICES, AND METHODS FOR PREVENTING POSTERIOR CAPSULE OPACIFICATION (PCO)

(71) Applicant: OHIO STATE INNOVATION FOUNDATION, Columbus, OH (US)

(72) Inventors: Hamid HAMEDI, Columbus, OH (US); Derek HANSFORD, Columbus, OH (US); Heather CHANDLER, Columbus, OH (US); Katelyn REILLY, Columbus, OH (US)

(21) Appl. No.: 18/957,393

(22) Filed: Nov. 22, 2024

Related U.S. Application Data
(60) Provisional application No. 63/601,832, filed on Nov. 22, 2023.

(51) Int. Cl. A61F 2/16; A61F 2/00

(52) U.S. Cl. CPC A61F 2/1613; A61F 2002/009; A61F 2002/1681; A61F 2002/1699

(57) ABSTRACT

Combinations of currently used polymer systems (e.g. PMMA, TRIS, HEMA, etc.) in intraocular lenses and contact lenses in addition to other polymers (e.g., PVA) were used to investigate the effects of surface chemistry, mechanical properties, curvature, and micropatterning on LEC migration, attachment, and/or subsequent epithelial-mesenchymal transition (EMT). As described herein, by modifying and/or patterning the edges of IOLs to be less stiff/more viscous and/or have defined curvature/topography, cell migration or response or control cell attachment/migration can be controlled, reducing or preventing posterior capsule opacification (PCO).

### CROSS-REFERENCE TO RELATED APPLICATIONS

This application claims benefit of priority of U.S. Provisional Application No. 63/601,832, filed Nov. 22, 2023, which is hereby incorporated herein by reference in its entirety.

### STATEMENT REGARDING FEDERALLY SPONSORED RESEARCH OR DEVELOPMENT

This invention was made with government support under R21 EY032226 awarded by the National Institutes of Health. The government has certain rights in the invention.

### BACKGROUND

Visually impairing cataracts are the leading cause of preventable blindness in the world. Presently, the treatment for cataracts involves the surgical removal of the opacified lens of the affected eye and replacement with an artificial intraocular lens, typically including an intraocular lens optic and haptics (“IOL”). Technological advances in cataract surgery with IOL implantation have made cataract surgery among the most effective surgical procedures.

The most frequent complication of cataract surgery is the opacification of the posterior capsule. Posterior capsule opacification (PCO) results from the migration of residual lens epithelial cells (“LEC”) between the IOL and the surface of the posterior capsule subsequent to cataract surgery. Once located between the IOL and the surface of the posterior capsule, the LECs can proliferate, leading to clouding of the normally clear posterior capsule. Clouding of the posterior capsule can decrease visual acuity if the opacification occurs within the visual axis.

Visually significant PCO requires an additional surgery to clear the visual axis of the eye. Presently, the most widely utilized procedure to clear the visual axis of PCO may be Neodymium:Yttrium-Aluminum-Garnet (“Nd:YAG”) laser capsulotomy. However, there may be substantial problems with this procedure such as IOL damage, postoperative intraocular pressure spikes, vitreous floaters, cystoid macular edema, retinal detachment, and IOL subluxation, or the like. Additionally, pediatric patients can be difficult to treat and a delay in treatment can lead to irreversible amblyopia. Many underdeveloped countries do not have access to a Nd:YAG laser and the cost can be prohibitive.

Prevention or inhibition of PCO fall into two broad categories: mechanical and pharmacological. Mechanical mechanisms to inhibit PCO have primarily focused on configuration of the IOL. Specifically, configuring the IOL to include a sharp posterior edge may provide a structural barrier to the migration of residual LECs between the IOL and the surface of the posterior capsule. However, while introduction of square edged IOLs appears to have reduced incidence of PCO, a review of Medicare claims data from 1993 to 2003 evidences that the number of laser capsulotomies performed in the United States to treat PCO in recipients of square edged IOL remains substantial.

Pharmacological mechanisms have also been proposed as a way to inhibit or prevent PCO. The effect of topical treatment with nonsteroidal anti-inflammatory drugs (“NSAIDs”) such as diclofenac and indomethacin after phacoemulsification do not appear to inhibit PCO. Additionally, the majority of pharmacological agents tested in-vitro for inhibition of migration and proliferation of LECs are antimetabolites and antimitotics which have not been used clinically because of their toxic side effects.

Also, sealed capsule irrigation devices have been investigates which function to allow selective irrigation of the lens capsule with LEC inhibiting pharmacologic agents. It is not clear, however, that use of such devices can be reduced to routine practice. Problems relating to incomplete seal of the lens capsule resulting in leakage of potentially toxic chemicals into the anterior chamber of the eye, rupture of the lens capsule during manipulation of the irrigation device, difficulty in assessing kill of LECs within the lens capsule, and an increase in the duration of routine cataract surgery limit the usefulness of such irrigation devices.

Accordingly, there remains a need for compositions, devices, and methods for preventing PCO in patients following IOL implantation.

### SUMMARY

Provided herein are improved polymers for use in intraocular lenses, including those currently used to treat cataracts. The polymer design, particularly viscoelastic properties and micropatterning, can prevent posterior capsular opacification (PCO), which occurs in up to 50% of surgical patients, and has an almost 100% incidence in pediatric patients. PCO occurs when remaining lens epithelial cells in the lens capsule after surgery migrate from the front of the lens capsule to the back or onto the implant and undergo epithelial to mesenchymal transition (EMT), causing scarring and impaired vision.

As described herein, combinations of currently used polymer systems (e.g. PMMA, TRIS, HEMA, etc.) in intraocular lenses and contact lenses in addition to other polymers (e.g., PVA) were used to investigate the effects of surface chemistry, mechanical properties, curvature, and micropatterning on LEC migration, attachment, and/or subsequent epithelial-mesenchymal transition (EMT). As described herein, by modifying and/or patterning the edges of IOLs to be less stiff/more viscous and/or have defined curvature/topography, cell migration or response or control cell attachment/migration can be controlled, reducing or preventing PCO.

Accordingly, provided herein are methods for decreasing the rate of posterior capsular opacification (PCO) observed in a population of subjects following implantation of an intraocular lens, wherein the intraocular lens comprises an optic disposed about an optical axis comprising an anterior surface and an opposing posterior surface, the surfaces configured to focus light when implanted within a capsular bag of an eye, and a support structure coupled to the optic. These methods can comprise: modifying an edge region of the optic to exhibit a surface chemistry, a mechanical property, a curvature, a micropattern, or a combination thereof that reduces the rate of lens epithelial cell (LEC) attachment, prevents LEC cells from undergoing an epithelial-mesenchymal transition (EMT), or a combination thereof following implantation in a subject in need thereof.

In some embodiments, modifying the edge region of the optic comprises increasing the hydrophobicity of the edge region of the optic. In certain embodiments, the edge region of the optic exhibits a water contact angle of from 80° to 110°.

In some embodiments, modifying the edge region of the optic comprises reducing the stiffness of the edge region of the optic. In certain embodiments, the edge region of the optic exhibits a Young's modulus of from 0.1 MPa to 6500 MPa.

In some embodiments, modifying the edge region of the optic comprises increasing the elasticity of the edge region of the optic. In certain embodiments, the edge region of the optic exhibits a storage modulus of from 10 Pa to 25,000 Pa.

In some embodiments, modifying the edge region of the optic comprises patterning the edge region of the optic to increase the root mean square (RMS) roughness of the edge region to from 0.1 nm to 50.0 nm.

In some embodiments, modifying the edge region of the optic comprises patterning the edge region of the optic to introduce a plurality of protrusions within the edge region. In certain embodiments, the plurality of protrusions comprise an array of microdots or micropillars. In certain embodiments, the microdots or micropillars have a largest average cross-sectional dimension of from 1 micron to 10 microns. In certain embodiments, the array of microdots or micropillars exhibits an average spacing of from 500 nm to 10 microns.

In some embodiments, modifying the edge region of the optic comprises modifying the curvature of the edge region of the optic. In certain embodiments, modifying the curvature of the edge region comprises introducing convex curvature within the edge region of the optic.

Also provided herein are intraocular lenses provided by these methods. For example, provided herein are intraocular lenses that comprise: an optic disposed about an optical axis comprising an anterior surface and an opposing posterior surface, the surfaces configured to focus light when implanted within a capsular bag of an eye; and a support structure coupled to the optic; wherein the optic comprises an edge region circumferentially disposed about a surface (e.g., the anterior surface) of the optic.

In some embodiments, the edge region can exhibit one or more of the following characteristics: a Young's modulus of from 0.1 MPa to 6500 MPa, a water contact angle of from 80° to 110°, a storage modulus of from 10 Pa to 25,000 Pa, a root mean square (RMS) roughness of the edge region to from 0.1 nm to 50.0 nm, or a combination thereof.

In some embodiments, the edge region can comprise an array of microdots or micropillars. In certain embodiments, the microdots or micropillars have a largest average cross-sectional dimension of from 1 micron to 10 microns. In certain embodiments, the array of microdots or micropillars exhibits an average spacing of from 500 nm to 10 microns.

### BRIEF DESCRIPTION OF THE DRAWINGS

FIG. 1 shows the chemical structures of (A) 2-hydroxyethyl methacrylate (HEMA), (B) 3-methacryloyloxypropyltris(trimethylsiloxy)silane (TRIS, also known as 3-[tris(trimethylsiloxoy)silyl]propyl methacrylate), and (C) 1,3-bis(3-(methacryloxy)propyl)-1,1,3,3-tetrakis(trimethylsiloxy)disiloxane (TRIS DIMER). The hydroxyl group of HEMA provided hydrophilicity (circle), trimethylsiloxy provided a hydrophobic functional group on TRIS (squares), and TRIS DIMER served as a crosslinker.

FIGS. 2A-2E illustrate Fourier transform infrared spectroscopy (FTIR) results for (2A and 2B) copolymer of TRIS:HEMA 1:1, (2C and 2D) crosslinking between polyTRIS chains, and (2E) attenuated total reflectance (ATR)-FTIR of TRIS:HEMA 1:1 and TRIS:HEMA 1:3.

FIG. 3 shows polymer stiffness data for synthesized polymers. Different ratios of monomers (TRIS:HEMA) and crosslinker (DIMER) were used to tune stiffness. Atomic force microscopy (AFM) imaging and force indentation theory were applied to measure stiffness.

FIG. 4 shows atomic force microscopy (AFM) height data indicating surface morphology of (panel A) PolyHEMA, (panel B) PolyTRIS, (panel C) TRIS:HEMA 1:3, and (panel D) TRIS:HEMA 1:1.

FIG. 5A is a plot showing the cell metabolism of human lens epithelial cells (hLEC) grown on synthesized polymer substrates (*p<0.01).

FIG. 5B shows the results of a live/dead assay for all samples at 24 and 48 h. Green fluorescence indicates viable cells (scale bar=750 μm).

FIG. 6A is a plot showing the ratio of cell density at 48/24 h, representing the ratio of cells that remained on the surface at 48 h compared to 24 h (*p<0.01).

FIG. 6B is a radar graph of the effect of normalized roughness (sample value/max value) and hydrophobicity (1 is the highest hydrophobicity and 0 is the lowest hydrophobicity of the samples) on normalized cell attachment (1 is the highest cell attachment and 0 is the lowest cell attachment).

FIG. 7A shows cell morphology on each polymer (I) pHEMA, (II) pTRIS, (III) T:H 1:3, and (IV) T:H 1:1 after 48 h (scale bar=300 μm).

FIG. 7B is a plot showing the aspect ratio of cells on different polymers after 48 h of culture (*p<0.01, NS, not significant).

FIG. 8A is a plot showing the results of an enzyme-linked immunosorbent assay (ELISA) for α-SMA expression on different polymers. Transforming growth factor (TGF)-β served as a positive control.

FIG. 8B is a plot showing the results of an enzyme-linked immunosorbent assay (ELISA) for β-catenin expression on different polymers. Transforming growth factor (TGF)-β served as a positive control.

FIG. 8C is a radar graph showing of the effect of normalized stiffness (the stiffness of each sample/the maximum stiffness among all samples) on normalized α-SMA and β-catenin expression, and cell aspect ratio (1 is the highest aspect ratio, spindle shaped cells, and 0 is the lowest normalized aspect ratio, fully rounded cells).

FIG. 9 illustrates how proper tuning of polymer properties, including stiffness, hydrophobicity, and surface roughness, impacts lens epithelial cell migration and attachment, and by extension posterior capsule opacification.

FIG. 10 illustrates how varying surface morphology and viscoelasticity impacts lens epithelial cell migration and attachment, and by extension posterior capsule opacification.

FIG. 11A schematically illustrates the imprinting technique used for transferring a micropattern from mold onto a coverslip (scale bar 150 μm).

FIG. 11B is a photo showing a micropatterned surface formed by the imprinting technique illustrated in FIG. 11A.

FIG. 12 is a schematic illustration of methods for preparing concave and convex surfaces.

FIG. 13A is a plot detailing the results of a rheological study evaluating the effect of freezing time on the viscoelasticity of PVA hydrogels.

FIG. 13B is a plot detailing the results of a rheological study evaluating the effect of the number of freeze-thaw cycles on the viscoelasticity of PVA hydrogels.

FIG. 13C is a plot detailing the results of a rheological study evaluating the effect of PVA concentration on the viscoelasticity of PVA hydrogels.

FIG. 13D is a plot showing frequency sweep results for three different hydrogels with the highest storage modulus (PVA 10, 24, 3), mid-range storage modulus (PVA 10,2, 1), and the lowest storage modulus (10, 0.25, 1) which were selected for further study.

FIG. 14A shows the cell response observed to difference curvatures: Panel I) Flat surface, Panel II) 1 mm concave surface, Panel III) 1 mm convex surface, Panel IV) 5 mm concave, and Panel V) 5 mm convex.

FIG. 14B shows a plot of the number of cells attached to polymeric surfaces having different curvatures. Image analysis using ImageJ was used to count number of attached hLECs on different curvatures.

FIG. 15A shows the impact of surface micropatterning on cell morphology. The micrographs herein show the results of Calcein AM staining of cells seeded on Panel I) microdots, Panel II) microlines, and Panel III) no pattern (control).

FIG. 15B is a plot showing the aspect ratio evaluation of cells in response to micropatterns. Image analysis using ImageJ was used to determine the average aspect ratio of cells on each surface.

FIG. 16A shows micrographs showing the results of Calcein AM staining and fluorescent microscopy imaging to visualize cell response to viscoelastic hydrogels (Scale bar=750 μm). Panel A) PVA 10, 0.25, 1 (lowest elasticity), Panel B) PVA 10, 2, 1 (medium elasticity), Panel C) PVA 10, 24, 3 (maximum elasticity), Panel D) Control (cells on the well plate—fully elastic surface)

FIG. 16B shows a plot of the number of cells attached to polymeric surfaces having varying viscoelasticity. Image analysis using ImageJ was used to count number of attached hLECs on different viscoelastic polymers compared to control.

FIG. 17 shows the results of a Western Blot assay for alpha-SMA and beta-catenin expression on different viscoelastic polymers.

FIG. 18A is a plot showing the results of ELISA assay for α-SMA expression on different viscoelastic polymers

FIG. 18B is a plot showing the results of ELISA assay for β-catenin expression on different viscoelastic polymers

FIG. 19 is a plot showing α-SMA expression of hLEC in response to micropatterns using ELISA assay.

FIG. 20 is a top view of an intraocular lens illustrating an anterior side of an optic and a peripheral region that includes a subsurface layer disposed below a surface of the intraocular lens.

FIG. 21 is a cross-sectional side view of the intraocular lens illustrated in FIG. 20 across a section 2-2.

### DETAILED DESCRIPTION

It is to be understood that the invention is not limited to the details of construction or process steps set forth in the following description. The invention is capable of other embodiments and of being practiced or being carried out in various ways using the teaching herein.

### Definitions

With respect to the terms used in this disclosure, the following definitions are provided.

Unless defined otherwise, all technical and scientific terms used herein have the same meaning as commonly understood by one of ordinary skill in the art to which the invention belongs. The polymer definitions are consistent with those disclosed in the Compendium of Polymer Terminology and Nomenclature, IUPAC Recommendations 2008, edited by: Richard G. Jones, Jaroslav Kahovec, Robert Stepto, Edward S. Wilks, Michael Hess, Tatsuki Kitayama, and W. Val Metanomski. All publications, patent applications, patents, and other references mentioned herein are incorporated by reference.

“A” or “an” entity refers to one or more of that entity; for example, “a polymer” refers to one or more of those compositions or at least one composition. As such, the terms “a” or “an”, “one or more” and “at least one” can be used interchangeably herein. Furthermore, the language “selected from the group consisting of” refers to one or more of the elements in the list that follows, including combinations of two or more of the elements.

“About” for the purposes of the present invention means that values or ranges of values may be expressed as from “about” one particular value to “about” another particular value. In the context of such a value or range of values “about” means plus or minus 10% of the value or range of values recited or claimed. When such a range of values is expressed, an embodiment includes from about one particular value to about the other particular value. Also, when such a range of values is expressed, another embodiment includes from one particular value to the other particular value and it will be understood that each particular value forms another embodiment.

“Active agent” for the purposes of this invention means any substance used to treat an ocular condition.

“Biocompatible” for the purposes of this invention means the ability of any material to perform the intended function of an embodiment of the invention without eliciting any undesirable local or systemic effects on the recipient and can include non-biodegradable materials such as: polyurethanes, polyisobutylene, polydimethylsiloxane elastomer, ethylene-alpha-olefin copolymers, acrylic polymers and copolymers, vinyl halide polymers and copolymers, polyvinyl esters, polyvinylidene chloride, polyacrylonitrile, polyvinyl ketones, polyvinyl aromatics such as polystyrene, copolymers of vinyl monomers and olefins such as ethylene-methyl methacrylate copolymers, acrylonitrile-styrene copolymers, acrylonitrile butadiene styrene resins, ethylene-vinyl acetate copolymers, polyamides such as Nylon 66 and polycaprolactone, alkyd resins, polycarbonates, polyoxyethylenes, polyimides, polyesters, epoxy resins, rayon-triacetate, cellophane, silicon rubber, silicon hydrogel, or biodegradable materials, as defined herein or combinations thereof.

“Biodegradable” for the purposes of this invention means the ability of any biocompatible material to breakdown within the physiological environment of the eye by one or more physical, chemical, or cellular processes at a rate consistent with providing structural or pharmaceutical barriers (or both) at a therapeutic level controllable by selection of a polymer or mixture of polymers (also referred to as polymeric materials), including, but not limited to: polylactide polymers (PLA), copolymers of lactic and glycolic acids (PLGA), polylactic acid-polyethylene oxide copolymers, poly(ε-caprolactone-co-L-lactic acid (PCL-LA), glycine/PLA copolymers, PLA copolymers involving polyethylene oxides (PEO), acetylated polyvinyl alcohol (PVA)/polycaprolactone copolymers, hydroxybutyrate-hydroxyvalerate copolymers, polyesters such as, but not limited to, aspartic acid and different aliphatic diols, poly(alkylene tartrates) and their copolymers with polyurethanes, polyglutamates with various ester contents and with chemically or enzymatically degradable bonds, other biodegradable nonpeptidic polyamides, amino acid polymers, polyanhydride drug carriers such as, but not limited to, poly(sebacic acid) (PSA), aliphatic-aromatic homopolymers, and poly(anhydride-co-imides), poly(phosphoesters) by matrix or pendant delivery systems, poly(phosphazenes), poly(iminocarbonate), crosslinked poly(ortho ester), hydroxylated polyester-urethanes, or the like. Hydrogels such as methylcellulose which act to release drug through polymer swelling are specifically excluded from the term.

“Intraocular” for the purposes of this invention means inside the eyeball (also referred to as an “eye”) and without limitation to the forgoing the anterior chamber, the ciliary sulcus, and posterior capsule of the eye; however, specifically excluding the external surface of the eye or intracorneal or intrasclera regions of the eye.

“Localized Region” for the purposes of this invention means substantially within a localized tissue region of the eye therapeutically affected (whether structurally or pharmaceutically) by implantation of embodiments of an intraocular implant.

“Ocular condition” for the purposes of this invention means a disease, ailment or condition which affects or involves the eye or any one of the parts or regions of the eye, such as PCO. The eye includes the eyeball and the tissues and fluids which constitute the eyeball, the periocular muscles (such as the oblique and rectus muscles) and the portion of the optic nerve which is within or adjacent to the eyeball.

“Posterior ocular condition” for the purposes of this invention means a disease, ailment or condition which affects or involves a posterior ocular region or site such as the choroid or sclera (in a position posterior to a plane through the posterior wall of the lens capsule), vitreous, vitreous chamber, retina, optic nerve (i.e. the optic disc), and blood vessels and nerve which vascularize or innervate a posterior ocular region or site.

“Substantially” for the purposes of this invention means largely, but not wholly, the same form, manner or degree and the particular element will have a range of configurations as a person of ordinary skill in the art would consider as having the same function or result. When a particular element is expressed as an approximation by use of the antecedent “substantially,” it will be understood that the particular element forms another embodiment.

“Suitable for implantation” for the purposes of this invention means with regard to embodiments of the intraocular implant dimensions which allow insertion or implantation without causing excessive tissue damage.

“Therapeutic level” for the purposes of this invention means an amount or a concentration of an active agent that has been locally delivered to an ocular region that is appropriate to reduce, inhibit, or prevent a symptom of an ocular condition.

As used herein, the term “(meth)” designates optional methyl substitution. Thus, a term such as “(meth)acrylates” denotes both methacrylates and acrylates.

The term “individual” includes humans and vertebrates.

“Target macromolecule” means the macromolecule being synthesized from the reactive monomer mixture comprising monomers, macromers, prepolymers, cross-linkers, initiators, additives, diluents, and the like.

The term “polymerizable compound” means a compound containing one or more polymerizable groups. The term encompasses, for instance, monomers, macromers, oligomers, prepolymers, cross-linkers, and the like.

“Polymerizable groups” are groups that can undergo chain growth polymerization, such as free radical and/or cationic polymerization, for example a carbon-carbon double bond which can polymerize when subjected to radical polymerization initiation conditions. Non-limiting examples of free radical polymerizable groups include (meth)acrylates, styrenes, vinyl ethers, (meth)acrylamides, N-vinyllactams, N-vinylamides, O-vinylcarbamates, O-vinylcarbonates, and other vinyl groups. Preferably, the free radical polymerizable groups comprise (meth)acrylate, (meth)acrylamide, N-vinyl lactam, N-vinylamide, and styryl functional groups, and mixtures of any of the foregoing. More preferably, the free radical polymerizable groups comprise (meth)acrylates, (meth)acrylamides, and mixtures thereof. The polymerizable group may be unsubstituted or substituted. For instance, the nitrogen atom in (meth)acrylamide may be bonded to a hydrogen, or the hydrogen may be replaced with alkyl or cycloalkyl (which themselves may be further substituted).

Any type of free radical polymerization may be used including but not limited to bulk, solution, suspension, and emulsion as well as any of the controlled radical polymerization methods such as stable free radical polymerization, nitroxide-mediated living polymerization, atom transfer radical polymerization, reversible addition fragmentation chain transfer polymerization, organotellurium mediated living radical polymerization, and the like.

A “monomer” is a mono-functional molecule which can undergo chain growth polymerization, and in particular, free radical polymerization, thereby creating a repeating unit in the chemical structure of the target macromolecule. Some monomers have di-functional impurities that can act as cross-linking agents. A “hydrophilic monomer” is also a monomer which yields a clear single phase solution when mixed with deionized water at 25° C. at a concentration of 5 weight percent. A “hydrophilic component” is a monomer, macromer, prepolymer, initiator, cross-linker, additive, or polymer which yields a clear single phase solution when mixed with deionized water at 25° C. at a concentration of 5 weight percent. A “hydrophobic component” is a monomer, macromer, prepolymer, initiator, cross-linker, additive, or polymer which is slightly soluble or insoluble in deionized water at 25° C.

A “macromolecule” is an organic compound having a number average molecular weight of greater than 1500, and may be reactive or non-reactive.

A “macromonomer” or “macromer” is a macromolecule that has one group that can undergo chain growth polymerization, and in particular, free radical polymerization, thereby creating a repeating unit in the chemical structure of the target macromolecule. Typically, the chemical structure of the macromer is different than the chemical structure of the target macromolecule, that is, the repeating unit of the macromer's pendent group is different than the repeating unit of the target macromolecule or its mainchain. The difference between a monomer and a macromer is merely one of chemical structure, molecular weight, and molecular weight distribution of the pendent group. As a result, and as used herein, the patent literature occasionally defines monomers as polymerizable compounds having relatively low molecular weights of about 1,500 Daltons or less, which inherently includes some macromers. In particular, monomethacryloxypropyl terminated mono-n-butyl terminated polydimethylsiloxane (molecular weight=500-1500 g/mol) (mPDMS) and mono-(2-hydroxy-3-methacryloxypropyl)-propyl ether terminated mono-n-butyl terminated polydimethylsiloxane (molecular weight=500-1500 g/mol) (OH-mPDMS) may be referred to as monomers or macromers. Furthermore, the patent literature occasionally defines macromers as having one or more polymerizable groups, essentially broadening the common definition of macromer to include prepolymers. As a result, and as used herein, di-functional and multi-functional macromers, prepolymers, and crosslinkers may be used interchangeably.

A “silicone-containing component” is a monomer, macromer, prepolymer, cross-linker, initiator, additive, or polymer in the reactive mixture with at least one silicon-oxygen bond, typically in the form of siloxy groups, siloxane groups, carbosiloxane groups, and mixtures thereof.

Examples of silicone-containing components which are useful in this invention may be found in U.S. Pat. Nos. 3,808,178, 4,120,570, 4,136,250, 4,153,641, 4,740,533, 5,034,461, 5,070,215, 5,244,981, 5,314,960, 5,331,067, 5,371,147, 5,760,100, 5,849,811, 5,962,548, 5,965,631, 5,998,498, 6,367,929, 6,822,016, 6,943,203, 6,951,894, 7,052,131, 7,247,692, 7,396,890, 7,461,937, 7,468,398, 7,538,146, 7,553,880, 7,572,841, 7,666,921, 7,691,916, 7,786,185, 7,825,170, 7,915,323, 7,994,356, 8,022,158, 8,163,206, 8,273,802, 8,399,538, 8,415,404, 8,420,711, 8,450,387, 8,487,058, 8,568,626, 8,937,110, 8,937,111, 8,940,812, 8,980,972, 9,056,878, 9,125,808, 9,140,825, 9,156,934, 9,170,349, 9,217,813, 9,244,196, 9,244,197, 9,260,544, 9,297,928, 9,297,929, and European Patent No. 080539. These patents are hereby incorporated by reference in their entireties.

A “polymer” is a target macromolecule composed of the repeating units of the monomers used during polymerization.

A “homopolymer” is a polymer made from one monomer; a “copolymer” is a polymer made from two or more monomers; a “terpolymer” is a polymer made from three monomers. A “block copolymer” is composed of compositionally different blocks or segments. Diblock copolymers have two blocks. Triblock copolymers have three blocks. “Comb or graft copolymers” are made from at least one macromer.

A “repeating unit” is the smallest group of atoms in a polymer that corresponds to the polymerization of a specific monomer or macromer.

An “initiator” is a molecule that can decompose into radicals which can subsequently react with a monomer to initiate a free radical polymerization reaction. A thermal initiator decomposes at a certain rate depending on the temperature; typical examples are azo compounds such as 1,1′-azobisisobutyronitrile and 4,4′-azobis(4-cyanovaleric acid), peroxides such as benzoyl peroxide, tert-butyl peroxide, tert-butyl hydroperoxide, tert-butyl peroxybenzoate, dicumyl peroxide, and lauroyl peroxide, peracids such as peracetic acid and potassium persulfate as well as various redox systems. A photo-initiator decomposes by a photochemical process; typical examples are derivatives of benzil, benzoin, acetophenone, benzophenone, camphorquinone, and mixtures thereof as well as various monoacyl and bisacyl phosphine oxides and combinations thereof.

A “cross-linking agent” is a di-functional or multi-functional monomer or macromer which can undergo free radical polymerization at two or more locations on the molecule, thereby creating branch points and a polymeric network. Common examples are ethylene glycol dimethacrylate, tetraethylene glycol dimethacrylate, trimethylolpropane trimethacrylate, methylene bisacrylamide, triallyl cyanurate, and the like.

A “prepolymer” is a reaction product of monomers which contains remaining polymerizable groups capable of undergoing further reaction to form a polymer.

A “polymeric network” is a cross-linked macromolecule that can swell but cannot dissolve in solvents. “Hydrogels” are polymeric networks that swell in water or aqueous solutions, typically absorbing at least 10 weight percent water. “Silicone hydrogels” are hydrogels that are made from at least one silicone-containing component with at least one hydrophilic component. Hydrophilic components may also include non-reactive polymers.

“Conventional hydrogels” refer to polymeric networks made from components without any siloxy, siloxane or carbosiloxane groups. Conventional hydrogels are prepared from reactive mixtures comprising hydrophilic monomers. Examples include 2-hydroxyethyl methacrylate (“HEMA”), N-vinyl pyrrolidone (“NVP”), N, N-dimethylacrylamide (“DMA”) or vinyl acetate. U.S. Pat. Nos. 4,436,887, 4,495,313, 4,889,664, 5,006,622, 5,039459, 5,236,969, 5,270,418, 5,298,533, 5,824,719, 6,420,453, 6,423,761, 6,767,979, 7,934,830, 8,138,290, and 8,389,597 disclose the formation of conventional hydrogels. Commercially available conventional hydrogels include, but are not limited to, etafilcon, genfilcon, hilafilcon, lenefilcon, nesofilcon, omafilcon, polymacon, and vifilcon, including all of their variants.

“Silicone hydrogels” refer to polymeric networks made from at least one hydrophilic component and at least one silicone-containing component. Examples of silicone hydrogels include acquafilcon, asmofilcon, balafilcon, comfilcon, delefilcon, enfilcon, falcon, fanfilcon, formofilcon, galyfilcon, lotrafilcon, narafilcon, riofilcon, samfilcon, senofilcon, somofilcon, and stenfilcon, including all of their variants, as well as silicone hydrogels as prepared in U.S. Pat. Nos. 4,659,782, 4,659,783, 5,244,981, 5,314,960, 5,331,067, 5,371,147, 5,998,498, 6,087,415, 5,760,100, 5,776,999, 5,789,461, 5,849,811, 5,965,631, 6,367,929, 6,822,016, 6,867,245, 6,943,203, 7,247,692, 7,249,848, 7,553,880, 7,666,921, 7,786,185, 7,956,131, 8,022,158, 8,273,802, 8,399,538, 8,470,906, 8,450,387, 8,487,058, 8,507,577, 8,637,621, 8,703,891, 8,937,110, 8,937,111, 8,940,812, 9,056,878, 9,057,821, 9,125,808, 9,140,825, 9,156,934, 9,170,349, 9,244,196, 9,244,197, 9,260,544, 9,297,928, 9,297,929 as well as WO 03/22321, WO 2008/061992, and US 2010/0048847. These patents are hereby incorporated by reference in their entireties.

An “interpenetrating polymeric network” comprises two or more networks which are at least partially interlaced on the molecular scale but not covalently bonded to each other and which cannot be separated without braking chemical bonds. A “semi-interpenetrating polymeric network” comprises one or more networks and one or more polymers characterized by some mixing on the molecular level between at least one network and at least one polymer. A mixture of different polymers is a “polymer blend.” A semi-interpenetrating network is technically a polymer blend, but in some cases, the polymers are so entangled that they cannot be readily removed.

The terms “reactive mixture” and “reactive monomer mixture” refer to the mixture of components (both reactive and non-reactive) which are mixed together and when subjected to polymerization conditions form the conventional or silicone hydrogels/polymers as well as lenses made therefrom. The reactive monomer mixture may comprise reactive components such as the monomers, macromers, prepolymers, cross-linkers, and initiators, additives such as wetting agents, release agents, polymers, dyes, light absorbing compounds such as UV absorbers, pigments, dyes and photochromic compounds, any of which may be reactive or non-reactive but are capable of being retained within the resulting biomedical device, as well as pharmaceutical and nutraceutical compounds, and any diluents. It will be appreciated that a wide range of additives may be added based upon the biomedical device which is made and its intended use. Concentrations of components of the reactive mixture are expressed as weight percentages of all components in the reactive mixture, excluding diluent. When diluents are used, their concentrations are expressed as weight percentages based upon the amount of all components in the reactive mixture and the diluent.

“Reactive components” are the components in the reactive mixture which become part of the chemical structure of the polymeric network of the resulting hydrogel by covalent bonding, hydrogen bonding, electrostatic interactions, the formation of interpenetrating polymeric networks, or any other means.

The term “multi-functional” refers to a component having two or more polymerizable groups. The term “mono-functional” refers to a component having one polymerizable group.

The terms “halogen” or “halo” indicate fluorine, chlorine, bromine, and iodine.

As used herein, the term “alkyl” refers to an unsubstituted or substituted linear or branched alkyl group containing the indicated number of carbon atoms. If no number is indicated, then alkyl (optionally including any substituents on alkyl) may contain 1 to 16 carbon atoms. Preferably, the alkyl group contains 1 to 10 carbon atoms, alternatively 1 to 7 carbon atoms, or alternatively 1 to 4 carbon atoms. Examples of alkyl include methyl, ethyl, propyl, isopropyl, butyl, iso-, sec- and tert-butyl, pentyl, hexyl, heptyl, 3-ethylbutyl, and the like. Examples of substituents on alkyl include 1, 2, or 3 groups independently selected from hydroxy, amino, amido, oxa, carboxy, alkyl carboxy, carbonyl, alkoxy, amido, carbamate, carbonate, halogen, phenyl, benzyl, and combinations thereof. “Alkylene” means a divalent alkyl group, such as —CH₂—, —CH₂CH₂—, —CH₂CH₂CH₂—, —CH₂CH(CH₃)CH₂—, and —CH₂CH₂CH₂CH₂—.

“Haloalkyl” refers to an alkyl group as defined above substituted with one or more halogen atoms, where each halogen is independently F, Cl, Br or I. A preferred halogen is F. Preferred haloalkyl groups contain 1-6 carbons, more preferably 1-4 carbons, and still more preferably 1-2 carbons. “Haloalkyl” includes perhaloalkyl groups, such as —CF₃— or —CF₂CF₃—. “Haloalkylene” means a divalent haloalkyl group, such as —CH₂CF₂—.

“Cycloalkyl” refers to an unsubstituted or substituted cyclic hydrocarbon containing the indicated number of ring carbon atoms. If no number is indicated, then cycloalkyl may contain 3 to 12 ring carbon atoms. Preferred are C₃-C₈ cycloalkyl groups, C₃-C₇ cycloalkyl, more preferably C₄-C₇ cycloalkyl, and still more preferably C₅-C₆ cycloalkyl. Examples of cycloalkyl include cyclopropyl, cyclobutyl, cyclopentyl, cyclohexyl, cycloheptyl and cyclooctyl. Examples of substituents on cycloalkyl include 1, 2, or 3 groups independently selected from alkyl, hydroxy, amino, amido, oxa, carbonyl, alkoxy, amido, carbamate, carbonate, halo, phenyl, benzyl, and combinations thereof. “Cycloalkylene” means a divalent cycloalkyl group, such as 1,2-cyclohexylene, 1,3-cyclohexylene, or 1,4-cyclohexylene.

“Heterocycloalkyl” refers to a cycloalkyl ring or ring system as defined above in which at least one ring carbon has been replaced with a heteroatom selected from nitrogen, oxygen, and sulfur. The heterocycloalkyl ring is optionally fused to or otherwise attached to other heterocycloalkyl rings and/or non-aromatic hydrocarbon rings and/or phenyl rings. Preferred heterocycloalkyl groups have from 5 to 7 members. More preferred heterocycloalkyl groups have 5 or 6 members. Heterocycloalkylene means a divalent heterocycloalkyl group.

“Aryl” refers to an unsubstituted or substituted aromatic hydrocarbon ring system containing at least one aromatic ring. The aryl group contains the indicated number of ring carbon atoms. If no number is indicated, then aryl may contain 6 to 14 ring carbon atoms. The aromatic ring may optionally be fused or otherwise attached to other aromatic hydrocarbon rings or non-aromatic hydrocarbon rings. Examples of aryl groups include phenyl, naphthyl, and biphenyl. Preferred examples of aryl groups include phenyl. Examples of substituents on aryl include 1, 2, or 3 groups independently selected from alkyl, hydroxy, amino, amido, oxa, carboxy, alkyl carboxy, carbonyl, alkoxy, amido, carbamate, carbonate, halo, phenyl, benzyl, and combinations thereof. “Arylene” means a divalent aryl group, for example 1,2-phenylene, 1,3-phenylene, or 1,4-phenylene.

“Heteroaryl” refers to an aryl ring or ring system, as defined above, in which at least one ring carbon atom has been replaced with a heteroatom selected from nitrogen, oxygen, and sulfur. The heteroaryl ring may be fused or otherwise attached to one or more heteroaryl rings, aromatic or nonaromatic hydrocarbon rings or heterocycloalkyl rings. Examples of heteroaryl groups include pyridyl, furyl, and thienyl. “Heteroarylene” means a divalent heteroaryl group.

“Alkoxy” refers to an alkyl group attached to the parent molecular moiety through an oxygen bridge. Examples of alkoxy groups include, for instance, methoxy, ethoxy, propoxy and isopropoxy. “Aryloxy” refers to an aryl group attached to a parent molecular moiety through an oxygen bridge. Examples include phenoxy. “Cyclic alkoxy” means a cycloalkyl group attached to the parent moiety through an oxygen bridge.

“Alkylamine” refers to an alkyl group attached to the parent molecular moiety through an —NH bridge. Alkyleneamine means a divalent alkylamine group, such as —CH₂CH₂NH—.

“Siloxanyl” refers to a structure having at least one Si—O—Si bond. Thus, for example, siloxanyl group means a group having at least one Si—O—Si group (i.e. a siloxane group), and siloxanyl compound means a compound having at least one Si—O—Si group. “Siloxanyl” encompasses monomeric (e.g., Si—O—Si) as well as oligomeric/polymeric structures (e.g., —[Si—O]ₙ—, where n is 2 or more). Each silicon atom in the siloxanyl group is substituted with independently selected R^{A} groups (where R^{A} is as defined in formula A options (b)-(i)) to complete their valence.

“Silyl” refers to a structure of formula R₃Si— and “siloxy” refers to a structure of formula R₃Si—O—, where each R in silyl or siloxy is independently selected from trimethylsiloxy, C₁-C₈ alkyl (preferably C₁-C₃ alkyl, more preferably ethyl or methyl), and C₃-C₈ cycloalkyl.

“Alkyleneoxy” refers to groups of the general formula -(alkylene-O)ₚ— or —(O-alkylene)ₚ-, wherein alkylene is as defined above, and p is from 1 to 200, or from 1 to 100, or from 1 to 50, or from 1 to 25, or from 1 to 20, or from 1 to 10, wherein each alkylene is independently optionally substituted with one or more groups independently selected from hydroxyl, halo (e.g., fluoro), amino, amido, ether, carbonyl, carboxyl, and combinations thereof. If p is greater than 1, then each alkylene may be the same or different and the alkyleneoxy may be in block or random configuration. When alkyleneoxy forms a terminal group in a molecule, the terminal end of the alkyleneoxy may, for instance, be a hydroxy or alkoxy (e.g., HO—[CH₂CH₂O]ₚ— or CH₃O—[CH₂CH₂O]ₚ—). Examples of alkyleneoxy include polymethyleneoxy, polyethyleneoxy, polypropyleneoxy, polybutyleneoxy, and poly(ethyleneoxy-co-propyleneoxy).

“Oxaalkylene” refers to an alkylene group as defined above where one or more non-adjacent CH₂ groups have been substituted with an oxygen atom, such as —CH₂CH₂OCH(CH₃)CH₂—. “Thiaalkylene” refers to an alkylene group as defined above where one or more non-adjacent CH₂ groups have been substituted with a sulfur atom, such as —CH₂CH₂SCH(CH₃)CH₂—.

Unless otherwise indicated, ratios, percentages, parts, and the like are by weight.

Unless otherwise indicated, numeric ranges, for instance as in “from 2 to 10,” are inclusive of the numbers defining the range (e.g., 2 and 10).

### Methods and Devices

Provided herein are methods for decreasing the rate of posterior capsular opacification (PCO) observed in a population of subjects following implantation of an intraocular lens, wherein the intraocular lens comprises an optic disposed about an optical axis comprising an anterior surface and an opposing posterior surface, the surfaces configured to focus light when implanted within a capsular bag of an eye, and a support structure coupled to the optic. These methods can comprise: modifying an edge region of the optic to exhibit a surface chemistry, a mechanical property, a curvature, a micropattern, or a combination thereof that reduces the rate of lens epithelial cell (LEC) attachment, prevents LEC cells from undergoing an epithelial-mesenchymal transition (EMT), or a combination thereof following implantation in a subject in need thereof.

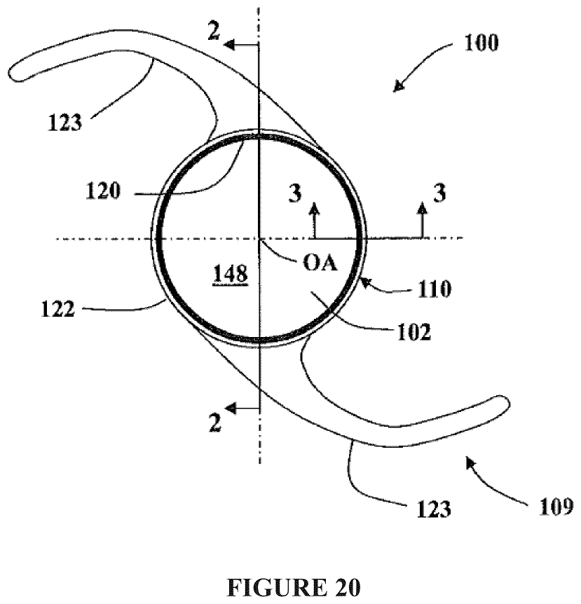

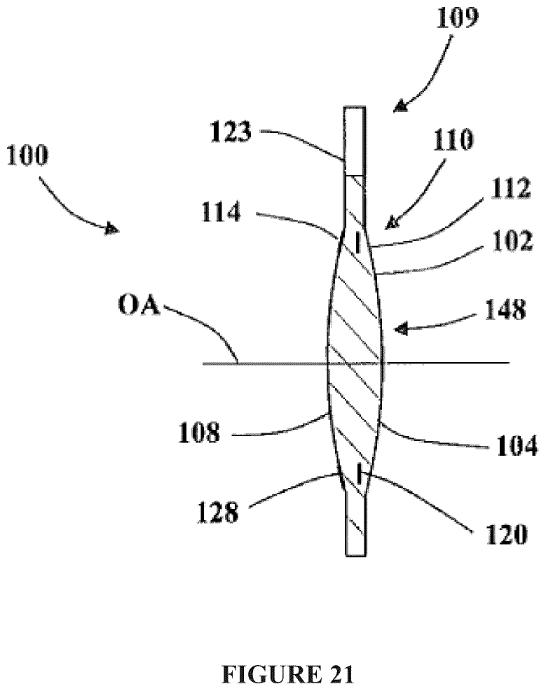

By way of example, referring to FIGS. 20-21, an intraocular lens 100 is illustrated that advantageously addresses the problem of unwanted cell migration (e.g., PCO). The intraocular lens 100 comprises an optic 102 disposed about an optical axis OA and has an anterior surface 104 and an opposing posterior surface 108. The surfaces 104, 108 are configured to focus light onto the retina of an eye into which the intraocular lens 100 is placed. The intraocular lens 100 further comprises a support structure 109 and an edge region 110 disposed about the optical axis OA that includes a top surface 112, a bottom surface 114. Optionally, the IOL can further include a subsurface layer 120 disposed between the top surface and bottom surfaces 112, 114. As discussed in greater detail below, the subsurface layer 120 may be configured to advantageously scatter or otherwise redirect incident light so as to reduce glare on the retina of an eye into which the intraocular lens 100 is placed. The subsurface layer 120 may also be configured for other uses such as for marking the intraocular lens 100 for identification or providing a practitioner information regarding the orientation or position of the intraocular lens 100.

The edge region 110 may also include an outer surface 122 that is disposed substantially parallel to the optical axis OA. The outer surface 122 may be straight, arcuate, or some combination thereof when viewed in cross-section in a plane congruent with the optical axis OA. In some embodiments, the outer surface 122 is also configured to reduce glare and/or PCO, for example, as disclosed in U.S. Pat. No. 6,884,262.

In the illustrated embodiment, the support structure 109 comprises two haptics 123. The haptics 123 may be used to center the intraocular lens 100 within the eye of a subject and are generally constructed to minimize damage to eye. In some embodiments, the support structure is more complex than that shown in FIG. 20. In certain embodiments, the support structure includes a structure that is configured to fill or substantially fill a capsular bag and/or to provide accommodative action.

As discussed above, edge region 110 of the optic can be modified to exhibit a surface chemistry, a mechanical property, a curvature, a micropattern, or a combination thereof that reduces the rate of lens epithelial cell (LEC) attachment, prevents LEC cells from undergoing an epithelial-mesenchymal transition (EMT), or a combination thereof. In some embodiments, the top surface 112 of the edge region can be modified to exhibit a surface chemistry, a mechanical property, a curvature, a micropattern, or a combination thereof that reduces the rate of lens epithelial cell (LEC) attachment, prevents LEC cells from undergoing an epithelial-mesenchymal transition (EMT), or a combination thereof. In some embodiments, the bottom surface 114 of the edge region can be modified to exhibit a surface chemistry, a mechanical property, a curvature, a micropattern, or a combination thereof that reduces the rate of lens epithelial cell (LEC) attachment, prevents LEC cells from undergoing an epithelial-mesenchymal transition (EMT), or a combination thereof. In some embodiments, the outer surface 122 of the edge region can be modified to exhibit a surface chemistry, a mechanical property, a curvature, a micropattern, or a combination thereof that reduces the rate of lens epithelial cell (LEC) attachment, prevents LEC cells from undergoing an epithelial-mesenchymal transition (EMT), or a combination thereof. In certain embodiments, two or more of the top surface 112, the bottom surface 114, the outer surface 122, can be modified to exhibit a surface chemistry, a mechanical property, a curvature, a micropattern, or a combination thereof that reduces the rate of lens epithelial cell (LEC) attachment, prevents LEC cells from undergoing an epithelial-mesenchymal transition (EMT), or a combination thereof. In some embodiments, the modified portion of the edge region 110 completely surrounds the central portion 148 of the optic 102.

In some embodiments, modifying the edge region of the optic comprises increasing the hydrophobicity of the edge region of the optic (e.g., the top surface 112, the bottom surface 114, the outer surface 122, or a combination thereof). This can be accomplished by covalent surface modification/functionalization of the edge region (or a portion thereof), or by fabrication of the edge region (or a portion thereof) from a polymeric matrix having the desired hydrophobicity.

In some embodiments, the modified portion of the edge region of the optic can exhibit a water contact angle of at least 80° (e.g., at least 81°, at least 82°, at least 83°, at least 84°, at least 85°, at least 86°, at least 87°, at least 88°, at least 89°, at least 90°, at least 91°, at least 92°, at least 93°, at least 94°, at least 95°, at least 96°, at least 97°, at least 98°, at least 99°, at least 100°, at least 101°, at least 102°, at least 103°, at least 104°, at least 105°, at least 106°, at least 107°, at least 108°, or at least 109°). In some embodiments, the modified portion of the edge region of the optic can exhibit a water contact angle of 110° or less (e.g., 109° or less, 108° or less, 107° or less, 1060 or less, 1050 or less, 1040 or less, 1030 or less, 1020 or less, 1010 or less, 1000 or less, 990 or less, 980 or less, 970 or less, 960 or less, 950 or less, 940 or less, 930 or less, 920 or less, 910 or less, 900 or less, 890 or less, 880 or less, 870 or less, 860 or less, 850 or less, 840 or less, 83° or less, 820 or less, or 810 or less).

The modified portion of the edge region of the optic can exhibit a water contact angle ranging from any of the minimum values described above to any of the maximum values described above. For example, in some embodiments, the modified portion of the edge region of the optic can exhibit a water contact angle of from 80° to 110° (e.g., from 900 to 1100).

In some embodiments, modifying the edge region of the optic comprises reducing the stiffness and/or increasing the elasticity of the edge region of the optic (e.g., the top surface 112, the bottom surface 114, the outer surface 122, or a combination thereof). This can be accomplished by fabrication of the edge region (or a portion thereof) from a polymeric matrix having the desired stiffness.

In some embodiments, the modified portion of the edge region of the optic can exhibit a Young's modulus of at least 0.1 MPa (e.g., at least 0.5 MPa, at least 1 MPa, at least 10 MPa, at least 50 MPa, at least 100 MPa, at least 500 MPa, at least 1000 MPa, at least 1500 MPa, at least 2000 MPa, at least 2500 MPa, at least 3000 MPa, at least 3500 MPa, at least 4000 MPa, at least 4500 MPa, at least 5000 MPa, at least 5500 MPa, or at least 6000 MPa). In some embodiments, the modified portion of the edge region of the optic can exhibit a Young's modulus of 6500 MPa or less (e.g., 6000 MPa or less, 5500 MPa or less, 5000 MPa or less, 4500 MPa or less, 4000 MPa or less, 3500 MPa or less, 3000 MPa or less, 2500 MPa or less, 2000 MPa or less, 1500 MPa or less, 1000 MPa or less, 500 MPa or less, 100 MPa or less, 50 MPa or less, 10 MPa or less, 1 MPa or less, or 0.5 MPa or less).

The modified portion of the edge region of the optic can exhibit a Young's modulus ranging from any of the minimum values described above to any of the maximum values described above. For example, in some embodiments, the modified portion of the edge region of the optic exhibits a Young's modulus of from 0.1 MPa to 6500 MPa.

In some embodiments, the modified portion of the edge region of the optic can exhibit a storage modulus of at least 10 Pa (e.g., at least 50 Pa, at least 100 Pa, at least 500 Pa, at least 1000 Pa, at least 2000 Pa, at least 2500 Pa, at least 3000 Pa, at least 4000 Pa, at least 5000 Pa, at least 6000 Pa, at least 7000 Pa, at least 8000 Pa, at least 9000 Pa, at least 10000 Pa, at least 11000 Pa, at least 12000 Pa, at least 13000 Pa, at least 14000 Pa, at least 15000 Pa, at least 16000 Pa, at least 17000 Pa, at least 18000 Pa, at least 19000 Pa, at least 20000 Pa, at least 21000 Pa, at least 22000 Pa, at least 23000 Pa, or at least 24000 Pa). In some embodiments, the modified portion of the edge region of the optic can exhibit a storage modulus of 25000 Pa or less (e.g., 24000 Pa or less, 23000 Pa or less, 22000 Pa or less, 21000 Pa or less, 20000 Pa or less, 19000 Pa or less, 18000 Pa or less, 17000 Pa or less, 16000 Pa or less, 15000 Pa or less, 14000 Pa or less, 13000 Pa or less, 12000 Pa or less, 11000 Pa or less, 10000 Pa or less, 9000 Pa or less, 8000 Pa or less, 7000 Pa or less, 6000 Pa or less, 5000 Pa or less, 4000 Pa or less, 3000 Pa or less, 2500 Pa or less, 2000 Pa or less, 1000 Pa or less, 500 Pa or less, 100 Pa or less, or 50 Pa or less).

The modified portion of the edge region of the optic can exhibit a storage modulus ranging from any of the minimum values described above to any of the maximum values described above. For example, in some embodiments, the modified portion of the edge region of the optic exhibits a storage modulus of from 10 Pa to 25,000 Pa.

In some embodiments, modifying the edge region of the optic comprises patterning the edge region of the optic to increase or decrease the root mean square (RMS) roughness of the edge region edge region of the optic (e.g., the top surface 112, the bottom surface 114, the outer surface 122, or a combination thereof).

In some embodiments, the modified portion of the edge region of the optic can exhibit a root mean square (RMS) roughness of at least 0.1 nm (e.g., at least 0.5 nm, at least 1 nm, at least 1.5 nm, at least 2 nm, at least 2.5 nm, at least 3 nm, at least 4 nm, at least 5 nm, at least 6 nm, at least 7 nm, at least 8 nm, at least 9 nm, at least 10 nm, at least 11 nm, at least 12 nm, at least 13 nm, at least 14 nm, at least 15 nm, at least 16 nm, at least 17 nm, at least 18 nm, at least 19 nm, at least 20 nm, at least 21 nm, at least 22 nm, at least 23 nm, at least 24 nm, at least 25 nm, at least 26 nm, at least 27 nm, at least 28 nm, or at least 29 nm). In some embodiments, the modified portion of the edge region of the optic can exhibit a root mean square (RMS) roughness of 30 nm or less (e.g., 29 nm or less, 28 nm or less, 27 nm or less, 26 nm or less, 25 nm or less, 24 nm or less, 23 nm or less, 22 nm or less, 21 nm or less, 20 nm or less, 19 nm or less, 18 nm or less, 17 nm or less, 16 nm or less, 15 nm or less, 14 nm or less, 13 nm or less, 12 nm or less, 11 nm or less, 10 nm or less, 9 nm or less, 8 nm or less, 7 nm or less, 6 nm or less, 5 nm or less, 4 nm or less, 3 nm or less, 2.5 nm or less, 2 nm or less, 1.5 nm or less, 1 nm or less, or 0.5 nm or less).

The modified portion of the edge region of the optic can exhibit a root mean square (RMS) roughness ranging from any of the minimum values described above to any of the maximum values described above. For example, in some embodiments, the modified portion of the edge region of the optic exhibits a root mean square (RMS) roughness of from 0.1 nm to 50.0 nm (e.g., from 1.0 nm to 30.0 nm, from 2.0 nm to 50.0 nm, from 2.0 nm to 30.0 nm, from 2.5 nm to 50.0 nm, or from 2.5 nm to 30.0 nm).

In some embodiments, modifying the edge region of the optic comprises patterning the edge region of the optic to introduce a plurality of protrusions within the edge region. In certain embodiments, the plurality of protrusions comprise an array of microdots or micropillars. In certain embodiments, the distal end of the micropillars can be tapered, narrow to a point, or exhibit curvature (convex or concave).

In certain embodiments, the microdots or micropillars can have a largest average cross-sectional dimension (perpendicular to the surface of the edge region) of at least 1 micron (e.g., at least 2 microns, at least 3 microns, at least 4 microns, at least 5 microns, at least 6 microns, at least 7 microns, at least 8 microns, or at least 9 microns). In certain embodiments, the microdots or micropillars have a largest average cross-sectional dimension of 10 microns or less (e.g., 9 microns or less, 8 microns or less, 7 microns or less, 6 microns or less, 5 microns or less, 4 microns or less, 3 microns or less, or 2 microns or less).

The microdots or micropillars can have a largest average cross-sectional dimension ranging from any of the minimum values described above to any of the maximum values described above. For example, in some embodiments, the microdots or micropillars can have a largest average cross-sectional dimension of from 1 micron to 10 microns.

In certain embodiments, the microdots or micropillars can have an average spacing (calculated as the average distance of a microdot or micropillar to its nearest neighbor microdot or micropillar) of at least 500 nm (e.g., at least 1 micron, at least 2 microns, at least 3 microns, at least 4 microns, at least 5 microns, at least 6 microns, at least 7 microns, at least 8 microns, or at least 9 microns). In certain embodiments, the microdots or micropillars can have an average spacing of 10 microns or less (e.g., 9 microns or less, 8 microns or less, 7 microns or less, 6 microns or less, 5 microns or less, 4 microns or less, 3 microns or less, 2 microns or less, or 1 micron or less).

The microdots or micropillars can have an average spacing ranging from any of the minimum values described above to any of the maximum values described above. For example, in some embodiments, the microdots or micropillars can have an average spacing of from 500 nm to 10 microns (e.g., from 1 micron to 10 microns).

### Components of IOLs and Methods of Making IOLs

The intraocular lenses described herein can be formed (in whole or in part) from suitable optically transparent materials such as, for example, a glass, allyl diglycol carbonate (ADC), a polycarbonate, a polyurethane, a thiourethane, a poly(meth)acrylate, a silicone hydrogel, or a combination thereof. Lenses can be fabricated from a reactive mixture comprising one or more reactive components using methods known in the art, as described in more detail below.

Examples of suitable families of reactive monomers include (meth)acrylates, styrenes, vinyl ethers, (meth)acrylamides, N-vinyl lactams, N-vinyl amides, N-vinyl imides, N-vinyl ureas, O-vinyl carbamates, O-vinyl carbonates, other hydrophilic vinyl compounds, and mixtures thereof.

Non-limiting examples of (meth)acrylate and (meth)acrylamide monomers include: acrylamide, N-isopropyl acrylamide, N,N-dimethylaminopropyl (meth)acrylamide, N,N-dimethyl acrylamide (DMA), 2-hydroxyethyl methacrylate (HEMA), 2-hydroxypropyl (meth)acrylate, 3-hydroxypropyl (meth)acrylate, 2,3-dihydroxypropyl (meth)acrylate, 2-hydroxybutyl (meth)acrylate, 3-hydroxybutyl (meth)acrylate, 4-hydroxybutyl (meth)acrylate, N-(2-hydroxyethyl) (meth)acrylamide, N,N-bis(2-hydroxyethyl) (meth)acrylamide, N-(2-hydroxypropyl) (meth)acrylamide, N,N-bis(2-hydroxypropyl) (meth)acrylamide, N-(3-hydroxypropyl) (meth)acrylamide, N-(2-hydroxybutyl) (meth)acrylamide, N-(3-hydroxybutyl) (meth)acrylamide, N-(4-hydroxybutyl) (meth)acrylamide, 2-aminoethyl (meth)acrylate, 3-aminopropyl (meth)acrylate, 2-aminopropyl (meth)acrylate, N-2-aminoethyl (meth)acrylamides), N-3-aminopropyl (meth)acrylamide, N-2-aminopropyl (meth)acrylamide, N,N-bis-2-aminoethyl (meth)acrylamides, N,N-bis-3-aminopropyl (meth)acrylamide), N,N-bis-2-aminopropyl (meth)acrylamide, glycerol methacrylate, polyethyleneglycol monomethacrylate, (meth)acrylic acid, vinyl acetate, acrylonitrile, and mixtures thereof.

Monomers may also be ionic, including anionic, cationic, zwitterions, betaines, and mixtures thereof. Non-limiting examples of such charged monomers include (meth)acrylic acid, N-[(ethenyloxy)carbonyl]-β-alanine (VINAL), 3-acrylamidopropanoic acid (ACA1), 5-acrylamidopentanoic acid (ACA2), 3-acrylamido-3-methylbutanoic acid (AMBA), 2-(methacryloyloxy)ethyl trimethylammonium chloride (Q Salt or METAC), 2-acrylamido-2-methylpropane sulfonic acid (AMPS), 1-propanaminium, N-(2-carboxyethyl)-N,N-dimethyl-3-[(1-oxo-2-propen-1-yl)amino]-, inner salt (CBT), 1-propanaminium, N,N-dimethyl-N-[3-[(1-oxo-2-propen-1-yl)amino]propyl]-3-sulfo-, inner salt (SBT), 3,5-Dioxa-8-aza-4-phosphaundec-10-en-1-aminium, 4-hydroxy-N,N,N-trimethyl-9-oxo-, inner salt, 4-oxide (9CI) (PBT), 2-methacryloyloxyethyl phosphorylcholine, 3-(dimethyl(4-vinylbenzyl)ammonio)propane-1-sulfonate (DMVBAPS), 3-((3-acrylamidopropyl)dimethylammonio)propane-1-sulfonate (AMPDAPS), 3-((3-methacrylamidopropyl)dimethylammonio)propane-1-sulfonate (MAMPDAPS), 3-((3-(acryloyloxy)propyl)dimethylammonio)propane-1-sulfonate (APDAPS), and 3-((3-(methacryloyloxy)propyl)dimethylammonio)propane-1-sulfonate (MAPDAPS).

Non-limiting examples of N-vinyl lactam and N-vinyl amide monomers include: N-vinyl pyrrolidone (NVP), N-vinyl-2-piperidone, N-vinyl-2-caprolactam, N-vinyl-3-methyl-2-caprolactam, N-vinyl-3-methyl-2-piperidone, N-vinyl-4-methyl-2-piperidone, N-vinyl-4-methyl-2-caprolactam, N-vinyl-3-ethyl-2-pyrrolidone, N-vinyl-4,5-dimethyl-2-pyrrolidone, N-vinyl acetamide (NVA), N-vinyl-N-methylacetamide (VMA), N-vinyl-N-ethyl acetamide, N-vinyl-N-ethyl formamide, N-vinyl formamide, N-vinyl-N-methylpropionamide, N-vinyl-N-methyl-2-methylpropionamide, N-vinyl-2-methylpropionamide, N-vinyl-N,N′-dimethylurea, 1-methyl-3-methylene-2-pyrrolidone, 1-methyl-5-methylene-2-pyrrolidone, 5-methyl-3-methylene-2-pyrrolidone; 1-ethyl-5-methylene-2-pyrrolidone, N-methyl-3-methylene-2-pyrrolidone, 5-ethyl-3-methylene-2-pyrrolidone, 1-N-propyl-3-methylene-2-pyrrolidone, 1-N-propyl-5-methylene-2-pyrrolidone, 1-isopropyl-3-methylene-2-pyrrolidone, 1-isopropyl-5-methylene-2-pyrrolidone, N-vinyl-N-ethyl acetamide, N-vinyl-N-ethyl formamide, N-vinyl formamide, N-vinyl isopropylamide, N-vinyl caprolactam, N-vinylimidazole, and mixtures thereof

Non-limiting examples of O-vinyl carbamates and O-vinyl carbonates monomers include N-2-hydroxyethyl vinyl carbamate and N-carboxy-β-alanine N-vinyl ester. Further examples of vinyl carbonate or vinyl carbamate monomers are disclosed in U.S. Pat. No. 5,070,215. Oxazolone monomers are disclosed in U.S. Pat. No. 4,910,277.

Other vinyl compounds include ethylene glycol vinyl ether (EGVE), di(ethylene glycol) vinyl ether (DEGVE), allyl alcohol, and 2-ethyl oxazoline.

The monomers may also be macromers or prepolymers of linear or branched poly(ethylene glycol), poly(propylene glycol), or statistically random or block copolymers of ethylene oxide and propylene oxide, having polymerizable moieties such as (meth)acrylates, styrenes, vinyl ethers, (meth)acrylamides, N-vinylamides, and the like. The macromers of these polyethers have one polymerizable group; the prepolymers may have two or more polymerizable groups. Other suitable hydrophilic monomers will be apparent to one skilled in the art.

In some embodiments, the reactive monomer mixture can comprise one or more silicone-containing component. Silicone-containing components suitable for use comprise one or more polymerizable compounds, where each compound independently comprises at least one polymerizable group, at least one siloxane group, and one or more linking groups connecting the polymerizable group(s) to the siloxane group(s). The silicone-containing components may, for instance, contain from 1 to 220 siloxane repeat units, such as the groups defined below. The silicone-containing component may also contain at least one fluorine atom.

The silicone-containing component may comprise: one or more polymerizable groups as defined above; one or more optionally repeating siloxane units; and one or more linking groups connecting the polymerizable groups to the siloxane units. The silicone-containing component may comprise: one or more polymerizable groups that are independently a (meth)acrylate, a styryl, a vinyl ether, a (meth)acrylamide, an N-vinyl lactam, an N-vinylamide, an O-vinylcarbamate, an O-vinylcarbonate, a vinyl group, or mixtures of the foregoing; one or more optionally repeating siloxane units; and one or more linking groups connecting the polymerizable groups to the siloxane units.

The silicone-containing component may comprise: one or more polymerizable groups that are independently a (meth)acrylate, a (meth)acrylamide, an N-vinyl lactam, an N-vinylamide, a styryl, or mixtures of the foregoing; one or more optionally repeating siloxane units; and one or more linking groups connecting the polymerizable groups to the siloxane units.

The silicone-containing component may comprise: one or more polymerizable groups that are independently a (meth)acrylate, a (meth)acrylamide, or mixtures of the foregoing; one or more optionally repeating siloxane units; and one or more linking groups connecting the polymerizable groups to the siloxane units.

Formula A. The silicone-containing component may comprise one or more polymerizable compounds of Formula A:

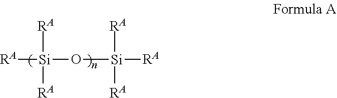

wherein:
at least one R^{A} is a group of formula R_{g}-L- wherein R_{g} is a polymerizable group and L is a linking group, and the remaining R^{A} are each independently:
   (a) R_{g}-L-,
   (b) C₁-C₁₆ alkyl optionally substituted with one or more hydroxy, amino, amido, oxa, carboxy, alkyl carboxy, carbonyl, alkoxy, amido, carbamate, carbonate, halo, phenyl, benzyl, or combinations thereof,
   (c) C₃-C₁₂ cycloalkyl optionally substituted with one or more alkyl, hydroxy, amino, amido, oxa, carbonyl, alkoxy, amido, carbamate, carbonate, halo, phenyl, benzyl, or combinations thereof,
   (d) a C₆-C₁₄ aryl group optionally substituted with one or more alkyl, hydroxy, amino, amido, oxa, carboxy, alkyl carboxy, carbonyl, alkoxy, amido, carbamate, carbonate, halo, phenyl, benzyl, or combinations thereof,
   (e) halo,
   (f) alkoxy, cyclic alkoxy, or aryloxy,
   (g) siloxy,
   (h) alkyleneoxy-alkyl or alkoxy-alkyleneoxy-alkyl, such as polyethyleneoxyalkyl, polypropyleneoxyalkyl, or poly(ethyleneoxy-co-propyleneoxyalkyl), or
   (i) a monovalent siloxane chain comprising from 1 to 100 siloxane repeat units optionally substituted with alkyl, alkoxy, hydroxy, amino, oxa, carboxy, alkyl carboxy, alkoxy, amido, carbamate, halo or combinations thereof; and
n is from 0 to 500 or from 0 to 200, or from 0 to 100, or from 0 to 20, where it is understood that when n is other than 0, n is a distribution having a mode equal to a stated value. When n is 2 or more, the SiO units may carry the same or different R^{A} substituents and if different R^{A} substituents are present, the n groups may be in random or block configuration.

In Formula A, three R^{A} may each comprise a polymerizable group, alternatively two R^{A} may each comprise a polymerizable group, or alternatively one R^{A} may comprise a polymerizable group.

Formula B. The silicone-containing component of formula A may be a mono-functional polymerizable compound of formula B:

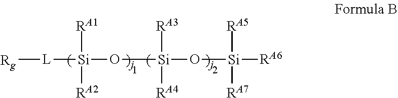

wherein:
Rg is a polymerizable group;
L is a linking group;
j1 and j2 are each independently whole numbers from 0 to 220, provided that the sum of j1 and j2 is from 1 to 220;
R^{A1}, R^{A2}, R^{A3}, R^{A4}, R^{A5}, and R^{A7} are independently at each occurrence C₁-C₆ alkyl, C₃-C₁₂ cycloalkyl, C₁-C₆ alkoxy, C₄-C₁₂ cyclic alkoxy, alkoxy-alkyleneoxy-alkyl, aryl (e.g., phenyl), aryl-alkyl (e.g., benzyl), haloalkyl (e.g., partially or fully fluorinated alkyl), siloxy, fluoro, or combinations thereof, wherein each alkyl in the foregoing groups is optionally substituted with one or more hydroxy, amino, amido, oxa, carboxy, alkyl carboxy, carbonyl, alkoxy, carbamate, carbonate, halo, phenyl, or benzyl, each cycloalkyl is optionally substituted with one or more alkyl, hydroxy, amino, amido, oxa, carbonyl, alkoxy, carbamate, carbonate, halo, phenyl, or benzyl and each aryl is optionally substituted with one or more alkyl, hydroxy, amino, amido, oxa, carboxy, alkyl carboxy, carbonyl, alkoxy, carbamate, carbonate, halo, phenyl, or benzyl;
and
R^{A6} is siloxy, C₁-C₈ alkyl (e.g., C₁-C₄ alkyl, or butyl, or methyl), or aryl (e.g., phenyl), wherein alkyl and aryl may optionally be substituted with one or more fluorine atoms.

Formula B-1. Compounds of formula B may include compounds of formula B-1, which are compounds of formula B wherein j1 is zero and j2 is from 1 to 220, or j2 is from 1 to 100, or j2 is from 1 to 50, or j2 is from 1 to 20, or j2 is from 1 to 5, or j2 is 1.

B-2. Compounds of formula B may include compounds of formula B-2, which are compounds of formula B wherein j1 and j2 are independently from 4 to 100, or from 4 to 20, or from 4 to 10, or from 24 to 100, or from 10 to 100.

B-3. Compounds of formulae B, B-1, and B-2 may include compounds of formula B-3, which are compounds of formula B, B-1, or B-2 wherein R^{A1}, R^{A2}, R^{A3}, and R^{A4} are independently at each occurrence C₁-C₆ alkyl or siloxy. Preferred alkyl are C₁-C₃ alkyl, or more preferably, methyl. Preferred siloxy is trimethylsiloxy.

B-4. Compounds of formulae B, B-1, B-2, and B-3 may include compounds of formula B-4, which are compounds of formula B, B-1, B-2, or B-3 wherein R^{A} and R^{A7} are independently alkoxy-alkyleneoxy-alkyl, preferably they are independently a methoxy capped polyethyleneoxyalkyl of formula CH₃O—[CH₂CH₂O]ₚ—CH₂CH₂CH₂, wherein p is a whole number from 1 to 50.

B-5. Compounds of formulae B, B-1, B-2, and B-3 may include compounds of formula B-5, which are compounds of formula B, B-1, B-2, or B-3 wherein R^{A} and R^{A7} are independently siloxy, such as trimethylsiloxy.

B-6. Compounds of formulae B, B-1, B-2, and B-3 may include compounds of formula B-6, which are compounds of formula B, B-1, B-2, or B-3 wherein R^{A} and R^{A7} are independently C₁-C₆ alkyl, alternatively C₁-C₄ alkyl, or alternatively, butyl or methyl.

B-7. Compounds of formulae B, B-1, B-2, B-3, B-4, B-5, and B-6 may include compounds of formula B-7, which are compounds of formula B, B-1, B-2, B-3, B-4, B-5, or B-6 wherein R^{A6} is C₁-C₈ alkyl, preferably C₁-C₆ alkyl, more preferably C₁-C₄ alkyl (for example methyl, ethyl, n-propyl, or n-butyl). More preferably R^{A6} is n-butyl.

B-8. Compounds of formulae B, B-1, B-2, B-3, B-4, B-5, B-6, and B-7, may include compounds of formula B-8, which are compounds of formula B, B-1, B-2, B-3, B-4, B-5, B-6, or B-7 wherein Rg comprises styryl, vinyl carbonate, vinyl ether, vinyl carbamate, N-vinyl lactam, N-vinylamide, (meth)acrylate, or (meth)acrylamide. Preferably, Rg comprises (meth)acrylate, (meth)acrylamide, or styryl. More preferably, Rg comprises (meth)acrylate or (meth)acrylamide. When Rg is (meth)acrylamide, the nitrogen group may be substituted with R^{A9}, wherein R^{A9} is H, C₁-C₈ alkyl (preferably C₁-C₄ alkyl, such as n-butyl, n-propyl, methyl or ethyl), or C₃-C₈ cycloalkyl (preferably C₅-C₆ cycloalkyl), wherein alkyl and cycloalkyl are optionally substituted with one or more groups independently selected from hydroxyl, amide, ether, silyl (e.g., trimethylsilyl), siloxy (e.g., trimethylsiloxy), alkyl-siloxanyl (where alkyl is itself optionally substituted with fluoro), aryl-siloxanyl (where aryl is itself optionally substituted with fluoro), and silyl-oxaalkylene- (where the oxaalkylene is itself optionally substituted with hydroxyl).

B-9. Compounds of formulae B, B-1, B-2, B-3, B-4, B-5, B-6, B-7, and B-8 may include compounds of formula B-9, which are compounds of formula B, B-1, B-2, B-3, B-4, B-5, B-6, B-7, or B-8 wherein the linking group comprises alkylene (preferably C₁-C₄ alkylene), cycloalkylene (preferably C₅-C₆ cycloalkylene), alkyleneoxy (preferably ethyleneoxy), haloalkyleneoxy (preferably haloethyleneoxy), amide, oxaalkylene (preferably containing 3 to 6 carbon atoms), siloxanyl, alkylenesiloxanyl, carbamate, alkyleneamine (preferably C₁-C₆ alkyleneamine), or combinations of two or more thereof, wherein the linking group is optionally substituted with one or more substituents independently selected from alkyl, hydroxyl, ether, amine, carbonyl, siloxy, and carbamate.

B-10. Compounds of formulae B, B-1, B-2, B-3, B-4, B-5, B-6, B-7, B-8, and B-9 may include compounds of formula B-10, which are compounds of formula B, B-1, B-2, B-3, B-4, B-5, B-6, B-7, B-8, or B-9 wherein the linking group is alkylene-siloxanyl-alkylene-alkyleneoxy-, or alkylene-siloxanyl-alkylene-[alkyleneoxy-alkylene-siloxanyl]_{q}-alkyleneoxy-, where q is from 1 to 50.

B-11. Compounds of formulae B, B-1, B-2, B-3, B-4, B-5, B-6, B-7, B-8, and B-9 may include compounds of formula B-11, which are compounds of formula B, B-1, B-2, B-3, B-4, B-5, B-6, B-7, B-8, or B-9 wherein the linking group is C₁-C₆ alkylene, preferably C₁-C₃ alkylene, more preferably n-propylene.

B-12. Compounds of formulae B, B-1, B-2, B-3, B-4, B-5, B-6, B-7, B-8, and B-9 may include compounds of formula B-12, which are compounds of formula B, B-1, B-2, B-3, B-4, B-5, B-6, B-7, B-8, or B-9 wherein the linking group is alkylene-carbamate-oxaalkylene. Preferably, the linking group is CH₂CH₂N(H)—C(═O)—O—CH₂CH₂—O—CH₂CH₂CH₂.

B-13. Compounds of formulae B, B-1, B-2, B-3, B-4, B-5, B-6, B-7, B-8, and B-9 may include compounds of formula B-13, which are compounds of formula B, B-1, B-2, B-3, B-4, B-5, B-6, B-7, B-8, or B-9 wherein the linking group is oxaalkylene. Preferably, the linking group is CH₂CH₂—O—CH₂CH₂CH₂.

B-14. Compounds of formulae B, B-1, B-2, B-3, B-4, B-5, B-6, B-7, B-8, and B-9 may include compounds of formula B-14, which are compounds of formula B, B-1, B-2, B-3, B-4, B-5, B-6, B-7, B-8, or B-9 wherein the linking group is alkylene-[siloxanyl-alkylene]_{q}-, where q is from 1 to 50. An example of such a linking group is: —(CH₂)₃-[Si(CH₃)₂—O—Si(CH₃)₂—(CH₂)₂]_{q}—.

B-15. Compounds of formulae B, B-1, B-2, B-3, B-4, B-5, B-6, B-7, B-8, and B-9 may include compounds of formula B-15, which are compounds of formula B, B-1, B-2, B-3, B-4, B-5, B-6, B-7, B-8, or B-9 wherein the linking group is alkyleneoxy-carbamate-alkylene-cycloalkylene-carbamate-oxaalkylene, wherein cycloalkylene is optionally substituted with or 1, 2, or 3 independently selected alkyl groups (preferably C₁-C₃ alkyl, more preferably methyl). An example of such a linking group is —[OCH₂CH₂]_{q}—OC(═O)—NH—CH₂-[1,3-cyclohexylene]-NHC(═O)O—CH₂CH₂—O—CH₂CH₂—, wherein the cyclohexylene is substituted at the 1 and 5 positions with 3 methyl groups.

B-16. Compounds of formulae B, B-1, B-2, B-3, B-4, B-5, B-6, B-7, B-8, and B-9 may include compounds of formula B-16, which are compounds of formula B, B-1, B-2, B-3, B-4, B-5, B-6, B-7, B-8, or B-9 wherein Rg comprises styryl and the linking group is alkyleneoxy wherein each alkylene in alkyleneoxy is independently optionally substituted with hydroxyl. An example of such a linking group is —O—(CH₂)₃—. Another example of such a linking group is —O—CH₂CH(OH)CH₂—O—(CH₂)₃—.

B-17. Compounds of formulae B, B-1, B-2, B-3, B-4, B-5, B-6, B-7, B-8, and B-9 may include compounds of formula B-17, which are compounds of formula B, B-1, B-2, B-3, B-4, B-5, B-6, B-7, B-8, or B-9 wherein Rg comprises styryl and the linking group is alkyleneamine. An example of such a linking group is —NH—(CH₂)₃—.

B-18. Compounds of formulae B, B-1, B-2, B-3, B-4, B-5, B-6, B-7, B-8, and B-9 may include compounds of formula B-18, which are compounds of formula B, B-1, B-2, B-3, B-4, B-5, B-6, B-7, B-8, or B-9 wherein the linking group is oxaalkylene optionally substituted with hydroxyl, siloxy, or silyl-alkyleneoxy (where the alkyleneoxy is itself optionally substituted with hydroxyl). An example of such a linking group is —CH₂CH(G)CH₂—O—(CH₂)₃—, wherein G is hydroxyl. In another example, G is R₃SiO— wherein two R groups are trimethylsiloxy and the third is C₁-C₈ alkyl (preferably C₁-C₃ alkyl, more preferably methyl) or the third is C₃-C₈ cycloalkyl. In a further example, G is R₃Si—(CH₂)₃O—CH₂CH(OH)CH₂—O—, wherein two R groups are trimethylsiloxy and the third is C₁-C₈ alkyl (preferably C₁-C₃ alkyl, more preferably methyl) or C₃-C₈ cycloalkyl. In a still further example, G is a polymerizable group, such as (meth)acrylate. Such compounds may function as crosslinkers.

B-19. Compounds of formulae B, B-1, B-2, B-3, B-4, B-5, B-6, B-7, B-8, and B-9 may include compounds of formula B-19, which are compounds of formula B, B-1, B-2, B-3, B-4, B-5, B-6, B-7, B-8, or B-9 wherein Rg comprises styryl and the linking group is amine-oxaalkylene optionally substituted with hydroxyl. An example of such a linking group is —NH—CH₂CH(OH)CH₂—O—(CH₂)₃—.

B-20. Compounds of formulae B, B-1, B-2, B-3, B-4, B-5, B-6, B-7, B-8, and B-9 may include compounds of formula B-20, which are compounds of formula B, B-1, B-2, B-3, B-4, B-5, B-6, B-7, B-8, or B-9 wherein Rg comprises styryl and the linking group is alkyleneoxy-carbamate-oxaalkylene. An example of such a linking group is —O—(CH₂)₂—N(H)C(═O)O—(CH₂)₂—O—(CH₂)₃—.

B-21. Compounds of formulae B, B-1, B-2, B-3, B-4, B-5, B-6, B-7, B-8, and B-9 may include compounds of formula B-21, which are compounds of formula B, B-1, B-2, B-3, B-4, B-5, B-6, B-7, B-8, or B-9 wherein the linking group is alkylene-carbamate-oxaalkylene. An example of such a linking group is —(CH₂)₂—N(H)C(═O)O—(CH₂)₂—O—(CH₂)₃—.

Formula C. Silicone-containing components of formulae A, B, B-1, B-2, B-3, B-4, B-5, B-6, B-7, B-8, B-9, B-10, B-11, B-12, B-13, B-14, B-15, B-18, and B-21 may include compounds of formula C, which are compounds of formula A, B, B-1, B-2, B-3, B-4, B-5, B-6, B-7, B-8, B-9, B-10, B-11, B-12, B-13, B-14, B-15, B-18, or B-21 having the structure:

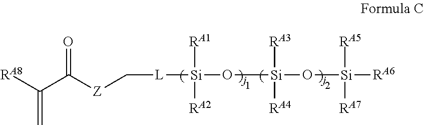

wherein
R^{A8} is hydrogen or methyl;
Z is O, S, or N(R^{A9}); and
L, j1, j2, R^{A1}, R^{A2}, R^{A3}, R^{A4}, R^{A5}, R^{A6}, R^{A7}, and R^{A9} are as defined in formula B or its various sub-formulae (e.g., B-1, B-2, etc.).

C-1. Compounds of formula C may include (meth)acrylates of formula C-1, which are compounds of formula C wherein Z is O.

C-2. Compounds of formula C may include (meth)acrylamides of formula C-2, which are compounds of formula C wherein Z is N(R^{A9}), and R^{A9} is H.

C-3. Compounds of formulae C may include (meth)acrylamides of formula C-3, which are compounds of formula C wherein Z is N(R^{A9}), and R^{A9} is C₁-C₈ alkyl that is unsubstituted or is optionally substituted as indicated above. Examples of R^{A9} include CH₃, —CH₂CH(OH)CH₂(OH), —(CH₂)₃-siloxanyl, —(CH₂)₃—SiR₃, and —CH₂CH(OH)CH₂—O—(CH₂)₃—SiR₃ where each R in the foregoing groups is independently selected from trimethylsiloxy, C₁-C₈ alkyl (preferably C₁-C₃ alkyl, more preferably methyl), and C₃-C₈ cycloalkyl. Further examples of R^{A9} include: —(CH₂)₃—Si(Me)(SiMe₃)₂, and —(CH₂)₃—Si(Me₂)-[O—SiMe₂]₁₋₁₀-CH₃.

Formula D. Compounds of formula C may include compounds of formula D:

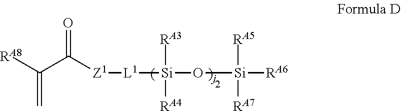

wherein
R^{A8} is hydrogen or methyl;
Z¹ is O or N(R^{A9});
L¹ is alkylene containing 1 to 8 carbon atoms, or oxaalkylene containing 3 to 10 carbon atoms, wherein L¹ is optionally substituted with hydroxyl; and
j2, R^{A3}, R^{A4}, R^{A5}, R^{A6}, R^{A7}, and R^{A9} are as defined above in formula B or its various sub-formulae (e.g., B-1, B-2, etc.).

D-1. Compounds of formula D may include compounds of formula D-1, which are compounds of formula D wherein L¹ is C₂-C₅ alkylene optionally substituted with hydroxyl. Preferably L¹ is n-propylene optionally substituted with hydroxyl.

D-2. Compounds of formula D may include compounds of formula D-2, which are compounds of formula D wherein L¹ is oxaalkylene containing 4 to 8 carbon atoms optionally substituted with hydroxyl. Preferably L¹ is oxaalkylene containing five or six carbon atoms optionally substituted with hydroxyl. Examples include —(CH₂)₂—O—(CH₂)₃—, and —CH₂CH(OH)CH₂—O—(CH₂)₃—.

D-3. Compounds of formulae D, D-1, and D-2 may include compounds of formula D-3, which are compounds of formula D, D-1, or D-2 wherein Z¹ is O.

D-4. Compounds of formulae D, D-1, and D-2 may include compounds of formula D-4, which are compounds of formula D, D-1, or D-2 wherein Z¹ is N(R^{A9}), and R^{A9} is H.

D-5. Compounds of formulae D, D-1, and D-2 may include compounds of formula D-5, which are compounds of formula D, D-1, or D-2 wherein Z¹ is N(R^{A9}), and R^{A9} is C₁-C₄ alkyl optionally substituted with 1 or 2 substituents selected from hydroxyl, siloxy, and C₁-C₆ alkyl-siloxanyl-.

D-6. Compounds of formulae D, D-1, D-2, D-3, D-4, and D-5 may include compounds of formula D-6, which are compounds of formula D, D-1, D-2, D-3, D-4, or D-5 wherein j2 is 1.

D-7. Compounds of formulae D, D-1, D-2, D-3, D-4, and D-5 may include compounds of formula D-7, which are compounds of formula D, D-1, D-2, D-3, D-4, or D-5 wherein j2 is from 2 to 220, or from 2 to 100, or from 10 to 100, or from 24 to 100, or from 4 to 20, or from 4 to 10.

D-8. Compounds of formulae D, D-1, D-2, D-3, D-4, D-5, D-6, and D-7 may include compounds of formula D-8, which are compounds of formula D, D-1, D-2, D-3, D-4, D-5, D-6, or D-7 wherein R^{A3}, R^{A4}, R^{A5}, R^{A6}, and R^{A7} are independently C₁-C₆ alkyl or siloxy. Preferably
R^{A3}, R^{A4}, R^{A5}, R^{A6}, and R^{A7} are independently selected from methyl, ethyl, n-propyl, n-butyl, and trimethylsiloxy. More preferably, R^{A3}, R^{A4}, R^{A5}, R^{A6}, and R^{A7} are independently selected from methyl, n-butyl, and trimethylsiloxy.

D-9. Compounds of formulae D, D-1, D-2, D-3, D-4, D-5, D-6, and D-7 may include compounds of formula D-9, which are compounds of formula D, D-1, D-2, D-3, D-4, D-5, D-6, or D-7 wherein R^{A3} and R^{A4} are independently C₁-C₆ alkyl (e.g., methyl or ethyl) or siloxy (e.g., trimethylsiloxy), and R^{A5}, R^{A6}, and R^{A7} are independently C₁-C₆ alkyl (e.g., methyl, ethyl, n-propyl, or n-butyl).

Formula E. The silicone-containing component may comprise a multi-functional silicone-containing component. Thus, for example, the silicone-containing component of formula A may comprise a bifunctional material of formula E:

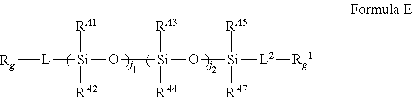

wherein
Rg, L, j1, j2, R^{A1}, R^{A2}, R^{A3}, R^{A4}, R^{A5}, and R^{A7} are as defined above for formula B or its various sub-formulae (e.g., B-1, B-2, etc.);
L² is a linking group; and
Rg¹ is a polymerizable group.

E-1. Compounds of formula E may include compounds of formula E-1, which are compounds of formula E wherein Rg and Rg¹ are each a vinyl carbonate of structure CH₂═CH—O—C(═O)—O— or structure CH₂═C(CH₃)—O—C(═O)—O—.

E-2. Compounds of formula E may include compounds of formula E-2, which are compounds of formula E wherein Rg and Rg¹ are each (meth)acrylate.

E-3. Compounds of formula E may include compounds of formula E-3, which are compounds of formula E wherein Rg and Rg¹ are each (meth)acrylamide, wherein the nitrogen group may be substituted with R^{A9} (wherein R^{A9} is as defined above).

E-4. Suitable compounds of formulae E, E-1, E-2, and E-3 include compounds of formula E-4, which are compounds of formula E, E-1, E-2, or E-3 wherein j1 is zero and j2 is from 1 to 220, or j2 is from 1 to 100, or j2 is from 1 to 50, or j2 is from 1 to 20.

E-5. Suitable compounds of formulae E, E-1, E-2, and E-3 include compounds of formula E-5, which are compounds of formula E, E-1, E-2, or E-3, wherein j1 and j2 are independently from 4 to 100.

E-6. Suitable compounds of formulae E, E-1, E-2, E-3, E-4, and E-5 include compounds of formula E-6, which are compounds of formula E, E-1, E-2, E-3, E-4, or E-5 wherein R^{A1}, R^{A2}, R^{A3}, R^{A4}, and R^{A5} are independently at each occurrence C₁-C₆ alkyl, preferably they are independently C₁-C₃ alkyl, or preferably, each is methyl.

E-7. Suitable compounds of formulae E, E-1, E-2, E-3, E-4, E-5, and E-6 include compounds of formula E-7, which are compounds of formula E, E-1, E-2, E-3, E-4, E-5, or E-6 wherein R^{A7} is alkoxy-alkyleneoxy-alkyl, preferably it is a methoxy capped polyethyleneoxyalkyl of formula CH₃O—[CH₂CH₂O]ₚ—CH₂CH₂CH₂, wherein p is a whole number from 1 to 50, or from 1 to 30, or from 1 to 10, or from 6 to 10.

E-8. Suitable compounds of formulae E, E-1, E-2, E-3, E-4, E-5, E-6, and E-7 include compounds of formula E-8, which are compounds of formula E, E-1, E-2, E-3, E-4, E-5, E-6, or E-7 wherein L comprises alkylene, carbamate, siloxanyl, cycloalkylene, amide, haloalkyleneoxy, oxaalkylene, or combinations of two or more thereof, wherein the linking group is optionally substituted with one or more substituents independently selected from alkyl, hydroxyl, ether, amine, carbonyl, and carbamate.

E-9. Suitable compounds of formulae E, E-1, E-2, E-3, E-4, E-5, E-6, E-7, and E-8 include compounds of formula E-9, which are compounds of formula E, E-1, E-2, E-3, E-4, E-5, E-6, E-7, or E-8 wherein L² comprises alkylene, carbamate, siloxanyl, cycloalkylene, amide, haloalkyleneoxy, oxaalkylene, or combinations of two or more thereof, wherein the linking group is optionally substituted with one or more substituents independently selected from alkyl, hydroxyl, ether, amine, carbonyl, and carbamate.

Examples of silicone-containing components suitable for use include, but are not limited to, compounds listed in the table below. Where the compounds in the table below include polysiloxane groups, the number of SiO repeat units in such compounds, unless otherwise indicated, is preferably from 3 to 100, more preferably from 3 to 40, or still more preferably from 3 to 20.

| | |
|---|---|
| 1 | mono-methacryloxypropyl terminated mono-n-butyl terminated polydimethylsiloxanes |
| | (mPDMS) (preferably containing from 3 to 15 SiO repeating units) |
| 2 | mono-acryloxypropyl terminated mono-n-butyl terminated polydimethylsiloxane |
| 3 | mono(meth)acryloxypropyl terminated mono-n-methyl terminated polydimethylsiloxane |
| 4 | mono(meth)acryloxypropyl terminated mono-n-butyl terminated polydiethylsiloxane |
| 5 | mono(meth)acryloxypropyl terminated mono-n-methyl terminated polydiethylsiloxane |
| 6 | mono(meth)acrylamidoalkylpolydialkylsiloxanes |
| 7 | mono(meth)acryloxyalkyl terminated mono-alkyl polydiarylsiloxanes |
| 8 | 3-methacryloxypropyltris(trimethylsiloxy)silane (TRIS) |
| 9 | 3-methacryloxypropylbis(trimethylsiloxy)methylsilane |
| 10 | 3-methacryloxypropylpentamethyl disiloxane |
| 11 | mono(meth)acrylamidoalkylpolydialkylsiloxanes |
| 12 | mono(meth)acrylamidoalkyl polydimethylsiloxanes |
| 13 | N-(2,3-dihydroxypropane)-N′-(propyl tetra(dimethylsiloxy) |
| | dimethylbutylsilane)acrylamide |
| 14 | N-[3-tris(trimethylsiloxy)silyl]-propyl acrylamide (TRIS-Am) |
| 15 | 2-hydroxy-3-[3-methyl-3,3-di(trimethylsiloxy)silylpropoxy]-propyl methacrylate |
| | (SiMAA) |
| 16 | 2-hydroxy-3-methacryloxypropyloxypropyl-tris(trimethylsiloxy)silane |
| 17 | mono-(2-hydroxy-3-methacryloxypropyl)-propyl ether terminated mono-n-butyl |
| | terminated polydimethylsiloxanes (OH-mPDMS) (containing from 4 to 30, or from 10 |
| | to 20, or from 4 to 8 SiO repeat units) |
| 18 | 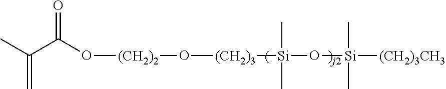 |
| 19 | 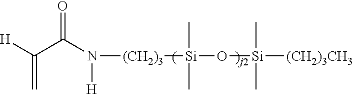 |
| 20 | 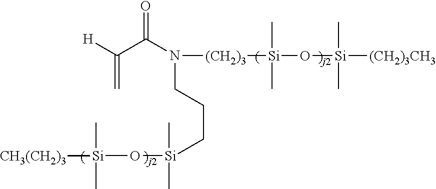 |
| 21 | 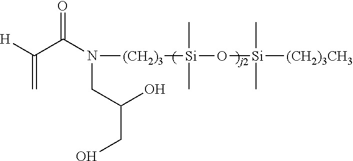 |
| 22 | 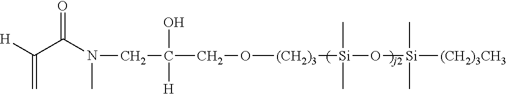 |
| 23 | 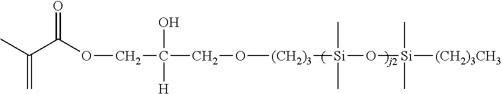 |
| 24 | 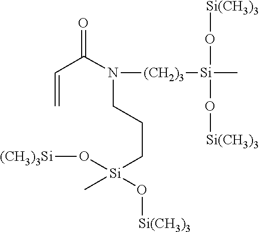 |

Additional non-limiting examples of suitable silicone-containing components are listed in the table below. Unless otherwise indicated, j2 where applicable is preferably from 1 to 100, more preferably from 3 to 40, or still more preferably from 3 to 15. In compounds containing j1 and j2, the sum of j1 and j2 is preferably from 2 to 100, more preferably from 3 to 40, or still more preferably from 3 to 15.

| | |
|---|---|
| 25 | 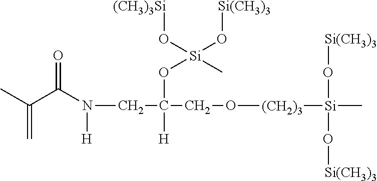 |
| 26 | 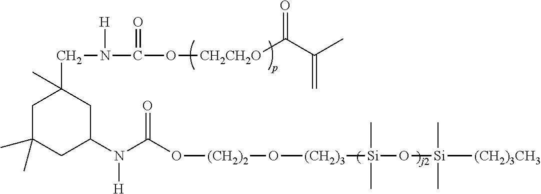 |
| | p is 1 to 10 |
| 27 | 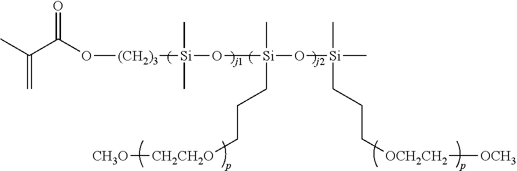 |
| | p is 5-10 |
| 28 | 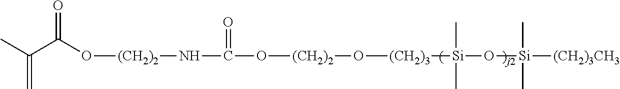 |
| 29 | 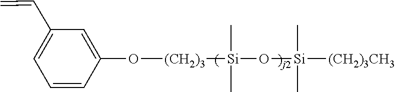 |
| 30 | 1,3-bis[4-(vinyloxycarbonyloxy)but-1-yl]tetramethyl-disiloxane |
| 31 | 3-(vinyloxycarbonylthio) propyl-[tris (trimethylsiloxy)silane] |
| 32 | 3-[tris(trimethylsiloxy)silyl] propyl allyl carbamate |
| 33 | 3-[tris(trimethylsiloxy)silyl] propyl vinyl carbamate |
| 34 | tris(trimethylsiloxy)silylstyrene (Styryl-TRIS) |
| 35 | 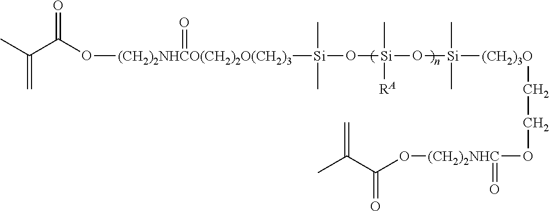 |
| | R^{A} = CH₃ (a) or CH₂CH₂CF₃ (b) or CH₂—(CH₂)₂—[OCH₂CH₂]₁₋₁₀—OCH₃ (c); a + b + c = n |
| 36 | 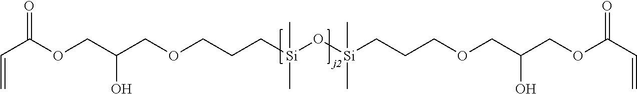 |
| 37 | 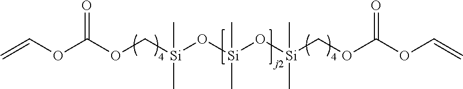 |
| 38 | 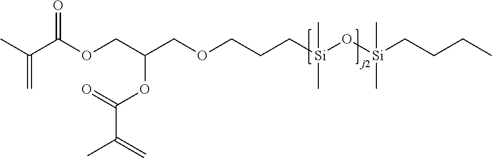 |
| 39 | 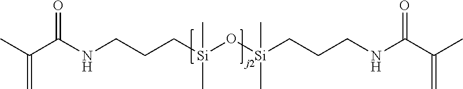 |
| 40 | 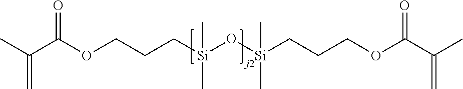 |
| 41 | 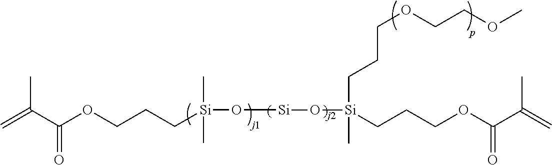 |
| | j1 = 80-90 |
| | j2 = 5-6 |
| | p = 7-8 |

In some examples, the silicone-containing component can comprise 1,3-bis(3-(methacryloxy)propyl)-1,1,3,3-tetrakis(trimethylsiloxy)disiloxane (TRIS DIMER).

Silicone-containing components may have an average molecular weight of from about 400 to about 4000 Daltons.

The silicone containing component(s) may be present in amounts up to about 95 weight %, or from about 10 to about 80 weight %, or from about 20 to about 70 weight %, based upon all reactive components of the reactive mixture (excluding diluents).

The reactive monomer mixture may include at least one polyamide. As used herein, the term “polyamide” refers to polymers and copolymers comprising repeating units containing amide groups. The polyamide may comprise cyclic amide groups, acyclic amide groups and combinations thereof and may be any polyamide known to those of skill in the art. Acyclic polyamides comprise pendant acyclic amide groups and are capable of association with hydroxyl groups. Cyclic polyamides comprise cyclic amide groups and are capable of association with hydroxyl groups.

Examples of suitable acyclic polyamides include polymers and copolymers comprising repeating units of Formulae G1 and G2:

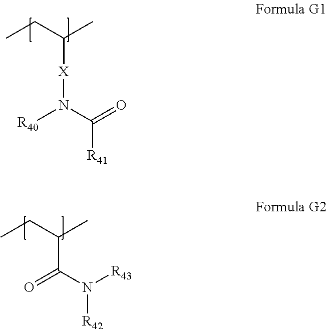

wherein X is a direct bond, —(CO)—, or —(CONHR₄₄)—, wherein R₄₄ is a C₁ to C₃ alkyl group; R₄₀ is selected from H, straight or branched, substituted or unsubstituted C₁ to C₄ alkyl groups; R₄₁ is selected from H, straight or branched, substituted or unsubstituted C₁ to C₄ alkyl groups, amino groups having up to two carbon atoms, amide groups having up to four carbon atoms, and alkoxy groups having up to two carbon groups; R₄₂ is selected from H, straight or branched, substituted or unsubstituted C₁ to C₄ alkyl groups; or methyl, ethoxy, hydroxyethyl, and hydroxymethyl; R₄₃ is selected from H, straight or branched, substituted or unsubstituted C₁ to C₄ alkyl groups; or methyl, ethoxy, hydroxyethyl, and hydroxymethyl; wherein the number of carbon atoms in R₄₀ and R₄₁ taken together is 8 or less, including 7, 6, 5, 4, 3, or less; and wherein the number of carbon atoms in R₄₂ and R₄₃ taken together is 8 or less, including 7, 6, 5, 4, 3, or less. The number of carbon atoms in R₄₀ and R₄₁ taken together may be 6 or less or 4 or less. The number of carbon atoms in R₄₂ and R₄₃ taken together may be 6 or less. As used herein substituted alkyl groups include alkyl groups substituted with an amine, amide, ether, hydroxyl, carbonyl or carboxy groups or combinations thereof.

R₄₀ and R₄₁ may be independently selected from H, substituted or unsubstituted C₁ to C₂ alkyl groups. X may be a direct bond, and R₄₀ and R₄₁ may be independently selected from H, substituted or unsubstituted C₁ to C₂ alkyl groups. R₄₂ and R₄₃ can be independently selected from H, substituted or unsubstituted C₁ to C₂ alkyl groups, methyl, ethoxy, hydroxyethyl, and hydroxymethyl.

The acyclic polyamides of the present invention may comprise a majority of the repeating units of Formula LV or Formula LVI, or the acyclic polyamides can comprise at least 50 mole percent of the repeating unit of Formula G or Formula G1, including at least 70 mole percent, and at least 80 mole percent. Specific examples of repeating units of Formula G and Formula G1 include repeating units derived from N-vinyl-N-methylacetamide, N-vinylacetamide, N-vinyl-N-methylpropionamide, N-vinyl-N-methyl-2-methylpropionamide, N-vinyl-2-methylpropionamide, N-vinyl-N,N′-dimethylurea, N, N-dimethylacrylamide, methacrylamide, and acyclic amides of Formulae G2 and G3:

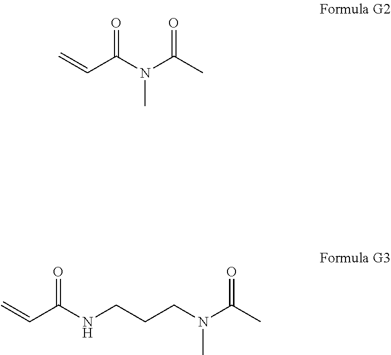

Examples of suitable cyclic amides that can be used to form the cyclic polyamides of include α-lactam, β-lactam, γ-lactam, δ-lactam, and ε-lactam. Examples of suitable cyclic polyamides include polymers and copolymers comprising repeating units of Formula G4:

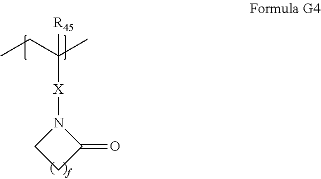

wherein R₄₅ is a hydrogen atom or methyl group; wherein f is a number from 1 to 10; wherein X is a direct bond, —(CO)—, or —(CONHR₄₆)—, wherein R₄₆ is a C₁ to C₃ alkyl group. In Formula LIX, f may be 8 or less, including 7, 6, 5, 4, 3, 2, or 1. In Formula G4, f may be 6 or less, including 5, 4, 3, 2, or 1. In Formula G4, f may be from 2 to 8, including 2, 3, 4, 5, 6, 7, or 8. In Formula LIX, f may be 2 or 3. When X is a direct bond, f may be 2. In such instances, the cyclic polyamide may be polyvinylpyrrolidone (PVP).

Cyclic polyamides may comprise 50 mole percent or more of the repeating unit of Formula G4, or the cyclic polyamides can comprise at least 50 mole percent of the repeating unit of Formula G4, including at least 70 mole percent, and at least 80 mole percent.

The polyamides may also be copolymers comprising repeating units of both cyclic and acyclic amides. Additional repeating units may be formed from monomers selected from hydroxyalkyl(meth)acrylates, alkyl(meth)acrylates, other hydrophilic monomers and siloxane substituted (meth)acrylates. Any of the monomers listed as suitable hydrophilic monomers may be used as comonomers to form the additional repeating units. Specific examples of additional monomers which may be used to form polyamides include 2-hydroxyethyl (meth)acrylate, vinyl acetate, acrylonitrile, hydroxypropyl (meth)acrylate, methyl (meth)acrylate and hydroxybutyl (meth)acrylate, dihydroxypropyl (meth)acrylate, polyethylene glycol mono(meth)acrylate, and the like and mixtures thereof. Ionic monomers may also be included. Examples of ionic monomers include (meth)acrylic acid, N-[(ethenyloxy)carbonyl]-β-alanine (VINAL, CAS #148969-96-4), 3-acrylamidopropanoic acid (ACA1), 5-acrylamidopentanoic acid (ACA2), 3-acrylamido-3-methylbutanoic acid (AMBA), 2-(methacryloyloxy)ethyl trimethylammonium chloride (Q Salt or METAC), 2-acrylamido-2-methylpropane sulfonic acid (AMPS), 1-propanaminium, N-(2-carboxyethyl)-N,N-dimethyl-3-[(1-oxo-2-propen-1-yl)amino]-, inner salt (CBT, carboxybetaine; CAS 79704-35-1), 1-propanaminium, N,N-dimethyl-N-[3-[(1-oxo-2-propen-1-yl)amino]propyl]-3-sulfo-, inner salt (SBT, sulfobetaine, CAS 80293-60-3), 3,5-Dioxa-8-aza-4-phosphaundec-10-en-1-aminium, 4-hydroxy-N,N,N-trimethyl-9-oxo-, inner salt, 4-oxide (9CI) (PBT, phosphobetaine, CAS 163674-35-9, 2-methacryloyloxyethyl phosphorylcholine, 3-(dimethyl(4-vinylbenzyl)ammonio)propane-1-sulfonate (DMVBAPS), 3-((3-acrylamidopropyl)dimethylammonio)propane-1-sulfonate (AMPDAPS), 3-((3-methacrylamidopropyl)dimethylammonio)propane-1-sulfonate (MAMPDAPS), 3-((3-(acryloyloxy)propyl)dimethylammonio)propane-1-sulfonate (APDAPS), 3-((3-(methacryloyloxy)propyl)dimethylammonio)propane-1-sulfonate (MAPDAPS).

The reactive monomer mixture may comprise both an acyclic polyamide and a cyclic polyamide or copolymers thereof. The acyclic polyamide can be any of those acyclic polyamides described herein or copolymers thereof, and the cyclic polyamide can be any of those cyclic polyamides described herein or copolymers thereof. The polyamide may be selected from the group polyvinylpyrrolidone (PVP), polyvinylmethylacetamide (PVMA), polydimethylacrylamide (PDMA), polyvinylacetamide (PNVA), poly hydroxyethyl (meth)acrylamide, polyacrylamide, and copolymers and mixtures thereof.

The total amount of all polyamides in the reactive mixture may be in the range of between 1 weight percent and about 35 weight percent, including in the range of about 1 weight percent to about 15 weight percent, and in the range of about 5 weight percent to about 15 weight percent, in all cases, based on the total weight of the reactive components of the reactive monomer mixture.

Without intending to be bound by theory, when used with a silicone hydrogel, the polyamide functions as an internal wetting agent. The polyamides may be non-polymerizable, and in this case, are incorporated into the silicone hydrogels as semi-interpenetrating networks. The polyamides are entrapped or physically retained within the silicone hydrogels. Alternatively, the polyamides may be polymerizable, for example as polyamide macromers or prepolymers, and in this case, are covalently incorporated into the silicone hydrogels. Mixtures of polymerizable and non-polymerizable polyamides may also be used.

When the polyamides are incorporated into the reactive monomer mixture they may have a weight average molecular weight of at least 100,000 daltons; greater than about 150,000; between about 150,000 to about 2,000,000 daltons; between about 300,000 to about 1,800,000 daltons. Higher molecular weight polyamides may be used if they are compatible with the reactive monomer mixture.

### Cross-Linking Agents

It is generally desirable to add one or more cross-linking agents, also referred to as cross-linking monomers, multi-functional macromers, and prepolymers, to the reactive mixture. The cross-linking agents may be selected from bifunctional crosslinkers, trifunctional crosslinkers, tetrafunctional crosslinkers, and mixtures thereof, including silicone-containing and non-silicone containing cross-linking agents. Non-silicone-containing cross-linking agents include ethylene glycol dimethacrylate (EGDMA), tetraethylene glycol dimethacrylate (TEGDMA), trimethylolpropane trimethacrylate (TMPTMA), triallyl cyanurate (TAC), glycerol trimethacrylate, methacryloxyethyl vinylcarbonate (HEMAVc), allyl methacrylate, methylene bisacrylamide (MBA), and polyethylene glycol dimethacrylate wherein the polyethylene glycol has a molecular weight up to about 5000 Daltons. The cross-linking agents are used in the usual amounts, e.g., from about 0.000415 to about 0.0156 mole per 100 grams of reactive Formulas in the reactive mixture. Alternatively, if the monomers and/or the silicone-containing components are multifunctional by molecular design or because of impurities, the addition of a cross-linking agent to the reactive mixture is optional. Examples of monomers and macromers which can act as the cross-linking agents and when present do not require the addition of an additional cross-linking agent to the reactive mixture include (meth)acrylate and (meth)acrylamide endcapped polyethers. Other cross-linking agents will be known to one skilled in the art.

It may be desirable to select crosslinking agents with similar reactivity to one or more of the other reactive components in the formulation. In some cases, it may be desirable to select a mixture of crosslinking agents with different reactivity in order to control some physical, mechanical or biological property of the resulting polymer. The structure and morphology of the polymer may also be influenced by the diluent(s) and cure conditions used.

Multifunctional silicone-containing components, including macromers, cross-linking agents, and prepolymers, may also be included to further increase the modulus and retain tensile strength. The silicone containing cross-linking agents may be used alone or in combination with other cross-linking agents. An example of a silicone containing component which can act as a cross-linking agent and, when present, does not require the addition of a crosslinking monomer to the reactive mixture includes α, ω-bismethacryloxypropyl polydimethylsiloxane.

Cross-linking agents that have rigid chemical structures and polymerizable groups that undergo free radical polymerization may also be used. Non-limiting examples of suitable rigid structures include cross-linking agents comprising phenyl and benzyl moieties, such are 1,4-phenylene diacrylate, 1,4-phenylene dimethacrylate, 2,2-bis(4-methacryloxyphenyl)-propane, 2,2-bis[4-(2-acryloxyethoxy)phenyl]propane, 2,2-bis[4-(2-hydroxy-3-methacryloxypropoxy)-phenyl]propane, and 4-vinylbenzyl methacrylate, and combinations thereof. Rigid crosslinking agents may be included in amounts between about 0.5 and about 15, or 2-10, 3-7 based upon the total weight of all of the reactive components. The physical and mechanical properties of the silicone hydrogels of the present invention may be optimized for a particular use by adjusting the components in the reactive mixture.

Non-limiting examples of silicone cross-linking agents also include the multi-functional silicone-containing components described above, such as compounds of Formula E (and its sub-formulae) and the multi-functional compounds shown in the tables above.

### Further Constituents

If desired, the reactive monomer mixture may contain additional components such as, but not limited to, diluents, initiators, UV absorbers, visible light absorbers, photochromic compounds, pharmaceuticals, nutraceuticals, antimicrobial substances, tints, pigments, copolymerizable dyes, nonpolymerizable dyes, release agents, and combinations thereof.

Classes of suitable diluents include alcohols having 2 to 20 carbon atoms, amides having to 20 carbon atoms derived from primary amines and carboxylic acids having 8 to 20 carbon atoms. The diluents may be primary, secondary, and tertiary alcohols.

Generally, the reactive components are mixed in a diluent to form a reactive mixture. Suitable diluents are known in the art. Suitable diluents are disclosed, for example, in WO 03/022321 and U.S. Pat. No. 6,020,445 the disclosure of which is incorporated herein by reference.

Specific diluents which may be used include 1-ethoxy-2-propanol, diisopropyl aminoethanol, isopropanol, 3,7-dimethyl-3-octanol, 1-decanol, 1-dodecanol, 1-octanol, 1-pentanol, 2-pentanol, 1-hexanol, 2-hexanol, 2-octanol, 3-methyl-3-pentanol, tert-amyl alcohol, tert-butanol, 2-butanol, 1-butanol, 2-methyl-2-pentanol, 2-propanol, 1-propanol, ethanol, 2-ethyl-1-butanol, (3-acetoxy-2-hydroxypropyloxy)-propylbis(trimethylsiloxy) methylsilane, 1-tert-butoxy-2-propanol, 3,3-dimethyl-2-butanol, tert-butoxyethanol, 2-octyl-1-dodecanol, decanoic acid, octanoic acid, dodecanoic acid, 2-(diisopropylamino)ethanol mixtures thereof and the like. Examples of amide diluents include N,N-dimethyl propionamide and dimethyl acetamide.

Preferred diluents include 3,7-dimethyl-3-octanol, 1-dodecanol, 1-decanol, 1-octanol, 1-pentanol, 1-hexanol, 2-hexanol, 2-octanol, 3-methyl-3-pentanol, 2-pentanol, t-amyl alcohol, tert-butanol, 2-butanol, 1-butanol, 2-methyl-2-pentanol, 2-ethyl-1-butanol, ethanol, 3,3-dimethyl-2-butanol, 2-octyl-1-dodecanol, decanoic acid, octanoic acid, dodecanoic acid, mixtures thereof and the like.

More preferred diluents include 3,7-dimethyl-3-octanol, 1-dodecanol, 1-decanol, 1-octanol, 1-pentanol, 1-hexanol, 2-hexanol, 2-octanol, 1-dodecanol, 3-methyl-3-pentanol, 1-pentanol, 2-pentanol, t-amyl alcohol, tert-butanol, 2-butanol, 1-butanol, 2-methyl-2-pentanol, 2-ethyl-1-butanol, 3,3-dimethyl-2-butanol, 2-octyl-1-dodecanol, mixtures thereof and the like. If a diluent is present, generally there are no particular restrictions with respect to the amount of diluent present. When diluent is used, the diluent may be present in an amount in the range of about 2 to about 70 weight percent, including in the range of about 5 to about 50 weight percent, and in the range of about 15 to about 40 weight percent, based on the total weight of the reactive mixtures (including reactive and nonreactive Formulas). Mixtures of diluents may be used.

A polymerization initiator may be used in the reactive mixture. The polymerization initiator may include, for instance, at least one of lauroyl peroxide, benzoyl peroxide, iso-propyl percarbonate, azobisisobutyronitrile, and the like, that generate free radicals at moderately elevated temperatures, and photoinitiator systems such as aromatic alpha-hydroxy ketones, alkoxyoxybenzoins, acetophenones, acylphosphine oxides, bisacylphosphine oxides, and a tertiary amine plus an α-diketone, mixtures thereof and the like. Illustrative examples of photoinitiators are 1-hydroxycyclohexyl phenyl ketone, 2-hydroxy-2-methyl-1-phenyl-propan-1-one, bis(2,6-dimethoxybenzoyl)-2,4-4-trimethylpentyl phosphine oxide (DMBAPO), bis(2,4,6-trimethylbenzoyl)-phenyl phosphine eoxide (Irgacure 819), 2,4,6-trimethylbenzyldiphenyl phosphine oxide and 2,4,6-trimethylbenzoyl diphenylphosphine oxide, benzoin methyl ester and a combination of camphorquinone and ethyl 4-(N,N-dimethylamino)benzoate. Diazo thermal initiators may also be used, such as azobisisobutyronitrile (AIBN), 2,2′-azobis(2-methylbutyronitrile) (AMBN) or similar compounds.

Commercially available visible light initiator systems include Irgacure® 819, Irgacure® 1700, Irgacure® 1800, Irgacure® 819, Irgacure® 1850 (all from Ciba Specialty Chemicals) and Lucrin® TPO initiator (available from BASF). Commercially available UV photoinitiators include Darocur® 1173 and Darocur® 2959 (Ciba Specialty Chemicals). These and other photoinitiators which may be used are disclosed in Volume III, Photoinitiators for Free Radical Cationic & Anionic Photopolymerization, 2nd Edition by J. V. Crivello & K. Dietliker; edited by G. Bradley; John Wiley and Sons; New York; 1998. The initiator is used in the reactive mixture in effective amounts to initiate photopolymerization of the reactive mixture, e.g., from about 0.1 to about 2 parts by weight per 100 parts of reactive monomer mixture. Polymerization of the reactive mixture can be initiated using the appropriate choice of heat or visible or ultraviolet light or other means depending on the polymerization initiator used. Alternatively, initiation can be conducted using e-beam without a photoinitiator. However, when a photoinitiator is used, the preferred initiators are bisacylphosphine oxides, such as bis(2,4,6-tri-methylbenzoyl)-phenyl phosphine oxide (Irgacure® 819) or a combination of 1-hydroxycyclohexyl phenyl ketone and bis(2,6-dimethoxybenzoyl)-2,4-4-trimethylpentyl phosphine oxide (DMBAPO).

### Curing of Reactive Mixtures and Manufacture of Lenses

The reactive mixtures may be formed by any of the methods known in the art, such as shaking or stirring, and used to form polymeric articles or devices by known methods. The reactive components are mixed together either with or without a diluent to form the reactive mixture.

For example, polymers may be prepared by mixing reactive components, and, optionally, diluent(s), with a polymerization initiator and curing by appropriate conditions to form a product that can be subsequently formed into the appropriate shape by lathing, cutting, and the like. Alternatively, the reactive mixture may be placed in a mold and subsequently cured into the appropriate article.

A method of making an intraocular lens may comprise: preparing a reactive monomer mixture; transferring the reactive monomer mixture onto a first mold; placing a second mold on top the first mold filled with the reactive monomer mixture; and curing the reactive monomer mixture by free radical copolymerization to form a polymer in the shape of the lens.

The reactive mixture may be cured via any known process for molding the reactive mixture in the production of lenses, including spincasting and static casting. Spincasting methods are disclosed in U.S. Pat. Nos. 3,408,429 and 3,660,545, and static casting methods are disclosed in U.S. Pat. Nos. 4,113,224 and 4,197,266. The lenses of this invention may be formed by the direct molding of polymers, which is economical, and enables precise control over the final shape of the hydrated lens. For this method, the reactive mixture is placed in a mold having the shape of the final desired polymer/hydrogel and the reactive mixture is subjected to conditions whereby the monomers polymerize, thereby producing a polymer in the approximate shape of the final desired product.

After curing, the lens may be subjected to extraction to remove unreacted components and release the lens from the lens mold. The extraction may be done using conventional extraction fluids, such organic solvents, such as alcohols or may be extracted using aqueous solutions.

Aqueous solutions are solutions which comprise water. The aqueous solutions of the present invention may comprise at least about 20 weight percent water, or at least about 50 weight percent water, or at least about 70 weight percent water, or at least about 95 weight percent water. Aqueous solutions may also include additional water soluble components such as inorganic salts or release agents, wetting agents, slip agents, pharmaceutical and nutraceutical compounds, combinations thereof and the like. Release agents are compounds or mixtures of compounds which, when combined with water, decrease the time required to release a contact lens from a mold, as compared to the time required to release such a lens using an aqueous solution that does not comprise the release agent. The aqueous solutions may not require special handling, such as purification, recycling or special disposal procedures.

Extraction may be accomplished, for example, via immersion of the lens in an aqueous solution or exposing the lens to a flow of an aqueous solution. Extraction may also include, for example, one or more of: heating the aqueous solution; stirring the aqueous solution; increasing the level of release aid in the aqueous solution to a level sufficient to cause release of the lens; mechanical or ultrasonic agitation of the lens; and incorporating at least one leaching or extraction aid in the aqueous solution to a level sufficient to facilitate adequate removal of unreacted components from the lens. The foregoing may be conducted in batch or continuous processes, with or without the addition of heat, agitation or both.

Application of physical agitation may be desired to facilitate leach and release. For example, the lens mold part to which a lens is adhered can be vibrated or caused to move back and forth within an aqueous solution. Other methods may include ultrasonic waves through the aqueous solution.

The lenses may be sterilized by known means such as, but not limited to, autoclaving.

The examples below are intended to further illustrate certain aspects of the materials and methods described herein, and is not intended to limit the scope of the claims

### EXAMPLES

### Example 1. Lens Epithelial Cell Response to Polymer Stiffness and Polymer Chemistry

### Summary

Posterior capsule opacification (PCO) is the most common complication of cataract surgery, and intraocular lens (IOL) implantation is the standard of care for cataract patients. Induction of postoperative epithelial-mesenchymal transition (EMT) in residual lens epithelial cells (LEC) is the main mechanism by which PCO forms. IOLs made with different materials have varying incidence of PCO. The aim of this Example was to study the interactions between human (h)LEC and polymer substrates. Polymers and copolymers of 2-hydroxyethyl methacrylate (HEMA) and 3-methacryloxypropyl tris(trimethylsiloxy)silane (TRIS) were synthesized and evaluated due to the clinical use of these materials as ocular biomaterials and implants. The chemical properties of the polymer surfaces were evaluated by contact angle, and polymer stiffness and roughness were measured using atomic force microscopy. In vitro studies showed the effect of polymer mechanical properties on the behavior of hLECs. Stiffer polymers increased α-smooth muscle actin expression and induced cell elongation. Hydrophobic and rough polymer surfaces increased cell attachment. These results demonstrate that attachment of hLECs on different surfaces is affected by surface properties in vitro, and evaluating these properties may be useful for investigating prevention of PCO.

### Introduction

The lens is an avascular biconvex structure, which is abundant in protein, particularly specialized crystallin proteins. The primary function of the ocular lens is to transmit and focus light onto the retina. The lens is surrounded by a basement membrane, referred to as the lens capsule, mainly comprised of collagen IV, providing an appropriate substrate for the internal epithelium to attach. These lens epithelial cells (LECs) normally reside as a monolayer on the anterior aspect of the lens and are not located posteriorly. Equatorially, the LEC undergo terminal differentiation to create the lens fibers, which make up the largest component of the lens. Cataract is the loss of lens transparency due to opacification of the lens tissue. Surgical removal of the lens fibers and intraocular lens (IOL) implantation into the remaining lens capsule is an effective technique to treat cataracts, and millions of procedures are performed every year. It is estimated that there are more than 3.5 million cases of cataract in the US annually, with 7 and 20 million cases in Europe and worldwide, respectively. Unfortunately, in the years following cataract surgery, a substantial proportion of cataract patients experience subsequent loss of vision due to a cellular response causing fibrosis, termed posterior capsule opacification (PCO). The incidence of this postoperative complication is even higher in young children (33%-72%).

During PCO formation, residual LECs aberrantly migrate to the posterior capsule, undergo epithelial-mesenchymal transition (EMT), and become fibrotic resulting in secondary vision loss. Although PCO can be treated with neodymium-doped yttrium aluminum garnet (Nd:YAG) laser capsulotomy, the procedure is costly and has increased risk of side effects including damage to the IOL, increase in intraocular pressure, posterior vitreous detachment, and retinal breaks or detachment. Moreover, Nd:YAG is difficult to perform in pediatric patients. Drug encapsulation onto or inside of IOLs has been studied as a technique to prevent PCO. However, there are several concerns about controlled and sustained release and ocular drug toxicity that still need to be addressed.

It is known that EMT is triggered by different growth factors in vivo such as transforming growth factor (TGF)-β, or via the physical microenvironment and the extracellular matrix (ECM). The mechanical properties of the ECM play an important role in regulating epithelial differentiation and EMT. Leight et al. reported the effect of mechanical properties and ECM stiffness on EMT regulation during tumor progression, noting that TGF-β signaling alone was not enough to trigger EMT at low ECM stiffness, indicating that biochemical changes promoting EMT were associated with stiffer ECM. Therefore, it is possible the stiffness and surface chemistry of IOLs could play a role in lens EMT, as well. In support of this, studies have indicated that the surface chemistry of the IOL affects LEC adhesion. One study demonstrated that the phenotype of adherent LECs on more hydrophobic surfaces changed and the cells underwent EMT.

Surface roughness also has important effects on cell adhesion, regardless of cell type and matrix materials. Recent in vitro studies showed submicron roughness could improve cell adhesion. One long-term study evaluating five different IOLs demonstrated that acrylate hydrophobic IOLs had a lower incidence of PCO, 3 and 5 years after implantation, compared to the most hydrophilic IOL evaluated. Further, surface energies have been shown to modulate cell adhesion and proliferation; surfaces with higher surface free energy have generally improved cell adhesion and spreading.

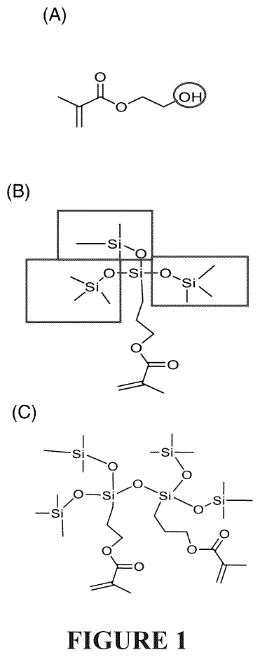

While surface chemistry has been investigated in the context of LEC response, results have been contradictory or inconclusive, indicating that other factors are likely influencing LEC response and driving EMT. We hypothesize that a combination of factors, including surface chemistry and substrate stiffness, modulate LEC adhesion and EMT. To test this hypothesis, we prepared a series of copolymers commonly used in ocular devices (including IOLs) and modulated surface free energy and stiffness. Synthesizing and crosslinking different homopolymers and copolymers enabled us to tune stiffness and hydrophilicity of the polymers to investigate LEC response on these substrates. In this Example, 3-methacryloxypropyl tris(trimethylsiloxy)silane (TRIS) and 2-hydroxyethyl methacrylate (HEMA) (FIG. 1) were chosen for polymerization due to their tolerability and their application in ocular research and clinical ophthalmic use. 1,3-bis(3-(methacryloxy)propyl)-1,1,3,3-tetrakis(trimethylsiloxy)disiloxane (DIMER) was used as a crosslinker to modulate polymer mechanical properties.

### Materials and Methods

Materials. Reagents 1,3-bis(3-methacryloxypropyl) tetrakis(trimethylsiloxy)-disiloxane (Gelest, Morrisville, PA, USA), 3-methacryloxypropyltris(trimethylsiloxy)silane (TRIS, hydrophobic siloxane) (Silar Labs, Riegelwood, NC, USA), 2-hydroxyethyl methacrylate (HEMA, hydrophilic acrylic) (Monomer-Polymer & Dajac Labs, Trevose, PA, USA), methyl methacrylate (MMA, hydrophobic acrylic) (Sigma-Aldrich, USA), isopropyl alcohol (Fisher Chemical, Canada), isooctane (Sigma Aldrich, Germany), methanol (Sigma-Aldrich, USA), mineral oil (Alfa Aesar, MA, USA), and 2,2′azobis-(2-methylbutyronitrile) (Vazo 67) (Monomer-Polymer & Dajac Labs, Trevose, PA, USA) were purchased and used as received. Other supplies included Dulbecco's Modified Eagle Medium (DMEM, Gibco, USA), fetal bovine serum (FBS, Gibco, USA), 3-(4,5-dimethylthiazol-2-yl)-5-(3-carboxymethoxyphenyl)-2-(4-sulfophenyl)-2H-tetrazolium (MTS, abeam, USA), Calcein AM (Invitrogen, Eugene, OR, USA), phosphate buffered saline (PBS, Sigma Life Science, UK), penicillin-streptomycin 100× (Corning, VA, USA), Live/dead assay kit (Invitrogen Carlsbad, CA, USA), and enzyme-linked immunosorbent assay (ELISA) kit (abeam; Waltham, MA, USA).

Polymer Synthesis. TRIS and HEMA monomers were copolymerized in a variety of molar ratios using free radical polymerization. Vazo 67 initiator was dissolved in 1.5% (w/w) isopropyl alcohol (TPA). Solutions were purged under nitrogen blanket for 2 min at room temperature. Then the reactor was placed in a hot oil bath at 70° C. and reacted for 6 h with stirring. Polymers were washed with deionized (DI) water and/or methanol to remove residual monomers. After drying, polymers were resuspended in IPA or isooctane at 15% w/w. Molar ratios of the monomers (TRIS:HEMA) and the polymer preparation procedures are summarized in Table 1. Different concentrations of crosslinker (DIMER) were also added to the pTRIS samples (1-4% w/w). Other polymers were screened at different ratios, but only the most relevant formulations are included below.

TABLE 1
Reaction solvent, washing solution, and
resuspension solvent used for polymers.

| | Reaction | Methanol/Water Wash | |
|---|---|---|---|
| Polymer | Solvent | Mixture (v/v ratio) | Resuspension |
| pTRIS | Isooctane | 100/0 | Isooctane |
| pHEMA | IPA | 0/100 | IPA |
| TRIS Dimer 1% | Isooctane | 100/0 | Isooctane |
| (TD 1%) | | | |
| TRIS Dimer 2% | Isooctane | 100/0 | Isooctane |
| (TD 1%) | | | |
| TRIS Dimer 3% | Isooctane | 100/0 | Isooctane |
| (TD 1%) | | | |
| TRIS Dimer 4% | Isooctane | 100/0 | Isooctane |
| (TD 1%) | | | |
| TRIS:HEMA 3:1 | IPA | 75/25 | Isooctane |
| (T:H 3:1) | | | |
| TRIS:HEMA 2:1 | IPA | 66.7/33.3 | Isooctane |
| (T:H 2:1) | | | |
| TRIS:HEMA 1:1 | IPA | 50/50 | Isooctane |
| (T:H 1:1) | | | |
| TRIS:HEMA 1:2 | IPA | 33.3/66.7 | IPA |
| (T:H 1:2) | | | |
| TRIS:HEMA 1:3 | IPA | 25/75 | IPA |
| (T:H 1:3) | | | |

Fourier transform infrared spectroscopy (FTIR)/attenuated total reflectance-FTIR (ATR-FTIR). Fourier transform infrared spectroscopy (FTIR) spectra were recorded on a Thermo Scientific Nicolet 6700 attenuated total reflectance (ATR)-FTIR equipped with a diamond crystal from 4000 to 600 cm⁻¹ with an average of 200 scans at 8 cm⁻¹ resolution.

Interactions between groups inside of the polymers were traced by this test using different polymer solutions. To investigate superficial functional groups, surface chemical analysis of the coated coverslips with TH 1:3 and TH 1:1 copolymer was performed using the ATR mode of FTIR. A germanium crystal was placed in contact with the surface of each polymer. When infrared light shines on the polymer surface, chemical groups in the surface of the polymer absorb specific wavelengths of light. Changes in the spectra are then used to identify the presence of specific functional groups.

Contact angle. For contact angle analysis, polymer solutions were spin-coated onto coverslips at 3000 rpm. Coated coverslips were placed on a hot plate for 5 min at 115° C. to allow for total evaporation of the solvent. Contact angle of a drop of distilled water or mineral oil were measured at room temperature by a ramé-hart goniometer (Succasunna, NJ, USA). Briefly, a drop of distilled water or mineral oil (5 μL) was placed on the surface of the dried samples and images of the drop profile after 5 s time lapse were converted by DROPimage Advanced software.

Stiffness measurement by atomic force microscopy (AFM). Atomic force microscope (AFM) force indentation experiments were performed using an Oxford Instruments AFM (MFP-3D Infinity, United Kingdom). A spherical shape tip was used (B500-NCH, radius 500 nm) with spring constant of 36.7 N m⁻¹. Briefly, indentations were performed on a 6 points×5 points grid, and the force curves versus indentation were generated. The Hertzian contact model (Equations (1) and (2)) was employed to relate force indentation and sample stiffness:

$$F = \frac{3}{4} {E}_{eff} \sqrt{{Rd}^{3}} \qquad ( Eq . 1 )$$

$$\frac{1}{{E}_{eff}} = \frac{1 - {v}_{tip}^{2}}{{E}_{tip}} + \frac{1 - {v}_{sample}^{2}}{{E}_{sample}} \qquad ( Eq . 2 )$$

ere F refers to force, d to indentation depth, E to Young's modulus, eff to effective, tip to indenter, sample to target, R to radius, and ν is Poisson's ratio. In this experiment tip properties came from the specification and νₛₐₘₚₗₑ was assumed to be equal to 0.33.
Eₜᵢₚ=867 GPa,
υₜᵢₚ=0.2,
υₛₐₘₚₗₑ=0.33
   AFM data were checked for any outliers using IBM SPSS software.

Surface roughness measurement by AFM. Surface microscopy was performed on each polymer sample using a cantilever with a sharp, tetrahedral tip (AC150TS-R₃, radius 7 nm) and a spring constant around 26 N m⁻¹. A small, characteristic 20 by 20 μm area of each sample was mapped using AFM in tapping mode. A significant effect of the sample composition on surface morphology was noted.

Each surface height map was comprised of 65,536 points and was used to calculate the roughness of the sample's surface. The average root mean square (RMS) roughness of each sample's surface was calculated using Equation (3):

$$Rq = \sqrt{\frac{1}{N} \sum {Z}^{{2}_{N}}} \qquad ( Eq . 3 )$$

where N represents the number of points measured and Z represents the height of the surface.

Cell metabolism and live/dead assay. Transformed hLEC (SRA01/04), characterized by Ibaraki et al. were used in this Example. For cell metabolism studies, 30 μL of each polymer solution was placed on 12 mm diameter coverslips and spin coated at 1200 rpm for 2 min. Coverslips were then transferred to a 24 well plate and left in a laminar flow hood overnight to allow for the solvent to evaporate. Once evaporated, each well was washed three times with sterile PBS. After removal of the last wash, plates were placed under UV light for sterilization prior to cell seeding. hLECs were seeded (1×10⁴) on each coverslip and cultured in medium (DMEM, 10% FBS, 1% penicillin-streptomycin). Wells seeded with cells without polymer served as the positive control and wells filled with media (no cells, no polymer) served as the negative control. Cell viability and cell growth were evaluated using an MTS assay after 24, 48, and 72 h of incubation, following the manufacturer's instructions. Optical density at 490 nm was determined using a Varioskan™ Lux multimode microplate reader, and SkanIT software (Thermo Fisher Scientific, Waltham, MA). Normalized cell activity (A) was evaluated by Equation (4):

$$A = \frac{( S - N )}{( P - N )} \times 100 \qquad ( Eq . 4 )$$

in this equation S, N, and P stand for sample, negative control, and positive control, respectively. Three replicates were used for each sample and control. These culture conditions were also applied to the live/dead assay, following the manufacturer's protocol. A fluorescent microscope (Invitrogen, EVOS 5000, WA, USA) was used to obtain three images of each sample after 24, 48, and 72 h incubation and ImageJ software was used for image analysis. Three replicates were used for each sample and control.

Cell attachment and cell morphology assay. To assess cell attachment and morphology, 1×10⁴ hLECs were seeded on polymer coated coverslips (12 mm diameter). Wells seeded with cells but without polymer served as the positive control. At 24 and 48 h, cells were stained with Calcein AM and imaged on a fluorescent microscope; images were analyzed using ImageJ. Three replicates were used for each sample and six images were taken for each sample. The change of average number of cells per area (10⁶ μm²) at 48 h compared to 24 h was determined for the cell attachment assay. Cell morphology aspect ratio (AR) and cell circularity were analyzed using ImageJ.

Enzyme-linked immunosorbent assay (ELISA). Experiments were performed using a 24-well plate. Polymer or PBS (control) was added to half of each well; a 5% sterile agarose solution was used to prevent polymer from covering the bottom of the entire well. The polymer solution was allowed to evaporate completely for 1 h followed by removal of the agarose. Cells (2.5×10⁴) were seeded into the center of each well and cultured for 48 h. Protein was isolated and quantified. Expression of α-SMA and β-catenin were measured in protein lysate using commercially available ELISAs, following manufacturer instructions. Each polymer was tested in triplicate. The positive TGF-β controls were cells that were treated with 10 ng mL⁻¹ TGF-beta 2 for 12 h and the negative control were untreated cells (cells in culture media).

Statistical analysis. Microsoft Excel software was used to determine p values by Student's t-test and analysis of variance (ANOVA), p<0.05 was considered statistically significant. SPSS was used to indicate outliers in the AFM data.

### Results and Discussion

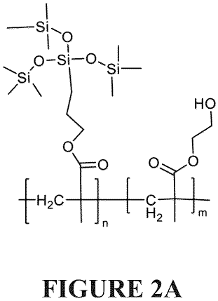

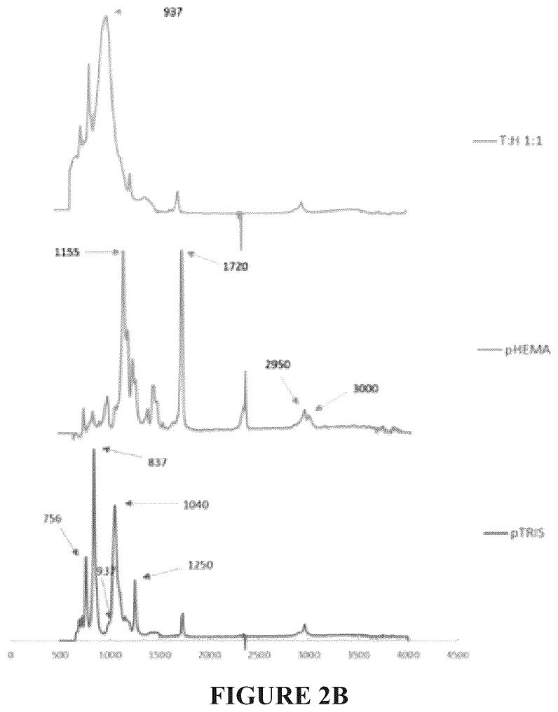

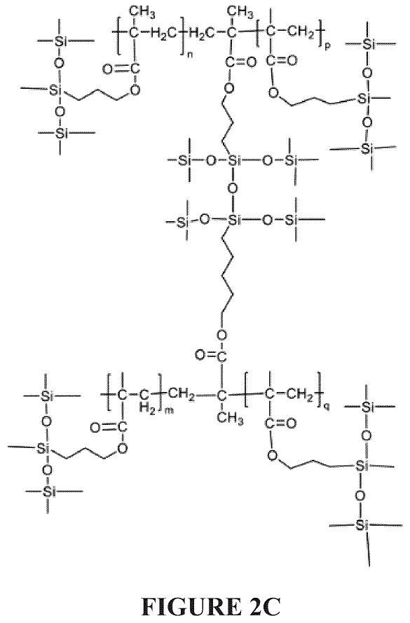

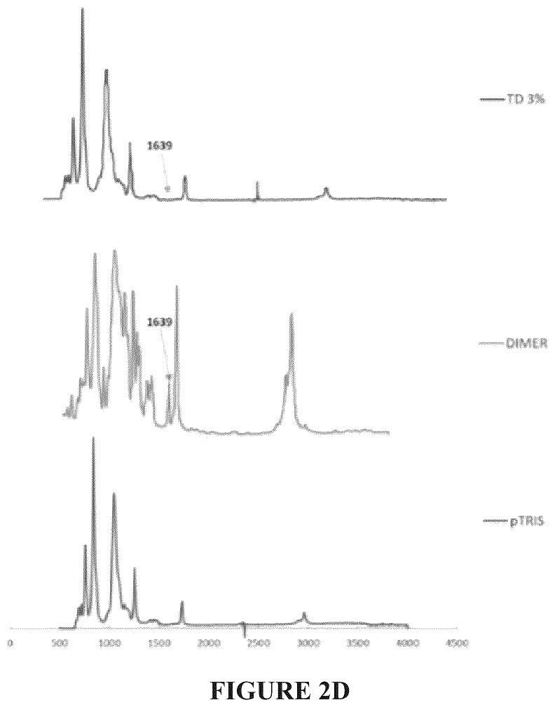

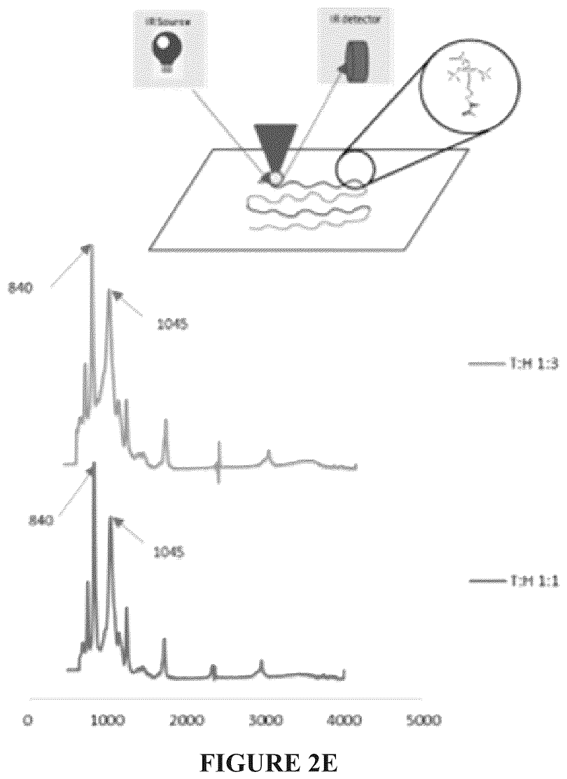

Fourier transform infrared spectroscopy (FTIR)/attenuated total reflectance-FTIR (ATR-FTIR). FTIR data and possible intermolecular interactions are presented in FIGS. 2A-2E. Characteristic peaks of polyHEMA (pHEMA) could be detected at 2950, 1720, and 1155 cm⁻¹ which are the hydroxyl stretch, carbonyl stretch, and —COH stretch, respectively. A sharp peak at 1250 cm⁻¹ represents the Si—CH₃ group, a characteristic peak for siloxane. A peak at 1040 cm⁻¹ demonstrates Si—O—Si asymmetric stretch, and the 837 and 756 cm⁻¹ peaks represent Si—(CH₃)₃ rocking mode. C═O stretching for an ester group could be detected in 1720 cm⁻¹ (FIGS. 2A-2B). Moreover, vinyl bonds associated with polyTRIS (pTRIS) at 937 cm⁻¹ and pHEMA at 3000 cm⁻¹ disappeared in TRIS:HEMA 1:1 (T:H 1:1), demonstrating successful copolymerization of TRIS with HEMA. A missing peak at 1639 cm⁻¹ for TRIS DIMER 3% (TD 3%) (FIGS. 2C-2D) indicates chemical crosslinking and removal of the DIMER vinyl bond. The presence of hydrophobic functional groups of TRIS repeat units on the surface of the copolymers (T:H 1:1 and T:H 1:3) were investigated with FTIR-ATR and shown in FIG. 2E. Peaks at 840 and 1045 cm⁻¹ are indications of Si—(CH₃)₃, and Si—O—Si functional groups of TRIS molecule. The lamellar sheet spatial arrangement of these block copolymers could be responsible for the presence of the TRIS repeat unit on the surface.

Contact angle. As presented in Table 2, pHEMA is a more hydrophilic sample with contact angle (θ<90°, where θ is the angle between a tangent to the liquid surface and the solid surface) while pTRIS is more hydrophobic with a larger water contact angle. Preparation of copolymers with these two monomers resulted in more hydrophobic properties, and TRIS surface chemistry was dominant in the copolymers after spin coating the samples for evaluation. As described by Emmanuel Pouget et al., vinyl group polymerization of siloxane containing monomers using azobis initiators could lead to block copolymerization. We hypothesize that spatial arrangement of these block copolymers could be a lamellar sheet in which the outer layer of this arrangement is formed by hydrophobic blocks or TRIS repeat units (as shown in FIG. 2E). Copolymerization of HEMA (H) with TRIS (T) decreased its lipophilic properties significantly (p<0.01). Crosslinking of pTRIS with DIMER (D) did not significantly change its contact a ngle (p=0.05). All the values for contact angle are an average of 10 replications±standard deviation.

TABLE 2
Contact angles of different polymer combinations to
quantify the effects of copolymerization of TRIS (T)
with HEMA (H) and crosslinking TRIS with dimer (D).

| Polymer | Water Contact Angle | Oil Contact Angle |
|---|---|---|
| pTRIS | 100.15 ± 0.03 | 52.46 ± 0.09 |
| T:H 3:1 | 101.10 ± 0.02 | 52.12 ± 0.01 |
| T:H 2:1 | 101.33 ± 0.01 | 49.65 ± 0.01 |
| T:H 1:1 | 100.78 ± 0.02 | 51.11 ± 0.01 |
| T:H 1:2 | 98.34 ± 0.04 | 49.43 ± 0.02 |
| T:H 1:3 | 101.25 ± 0.02 | 48.33 ± 0.01 |
| pHEMA | 53.90 ± 0.03 | 18.68 ± 0.01 |
| TD 1% | 102.29 ± 0.02 | 49.14 ± 0.02 |
| TD 2% | 99.79 ± 0.02 | 48.61 ± 0.01 |
| TD 3% | 100.23 ± 0.02 | 47.23 ± 0.02 |
| TD 4% | 102.00 ± 0.02 | 48.93 ± 0.08 |

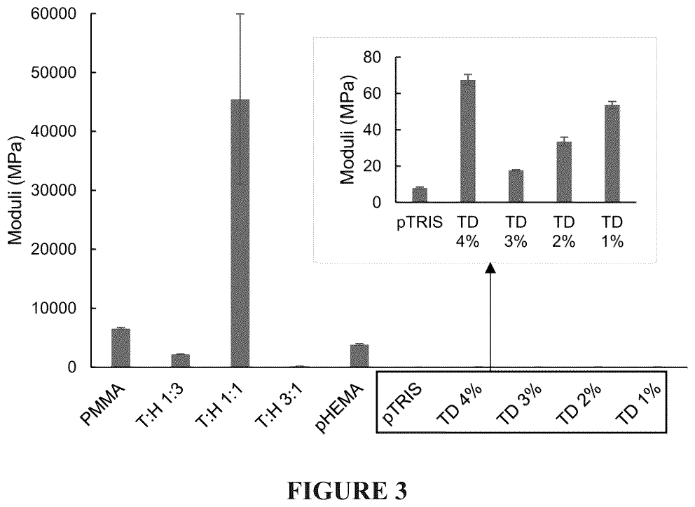

Stiffness and surface roughness. As presented in FIG. 3, the copolymer of TRIS:HEMA at 1:1 molar ratio (T:H 1:1) had a significantly higher stiffness (p<0.01) than other polymer samples (˜55,000 MPa). Poly(methyl methacrylate) (PMMA) control, which is a prevalent polymer in IOLs, had a stiffness of 8000 MPa, which was significantly (p<0.01) lower than T:H 1:1. pHEMA had a stiffness approximately half that of PMMA (˜4500 MPa). Crosslinking of pTRIS increased its modulus significantly (p<0.01) from 9.4 MPa (pTRIS) to 80 MPa by crosslinking with 4% DIMER (TD 4%). Of the polymers prepared and evaluated, pTRIS had the lowest stiffness, pHEMA and T:H 1:3 had intermediate stiffnesses, and T:H 1:1 had the highest stiffness; accordingly, these polymers were therefore selected for further evaluation.

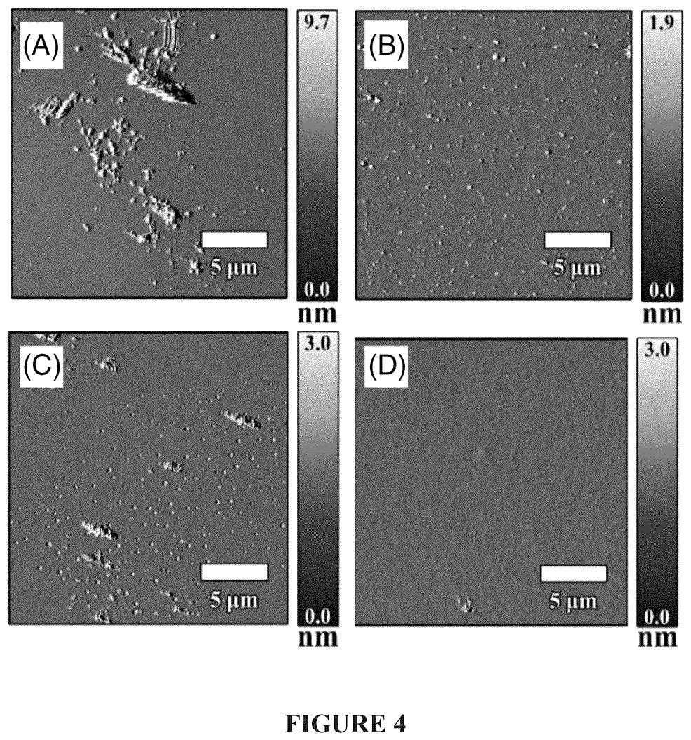

Surface roughness is a surface property that has an important effect on cell behavior and attachment. Sample surface roughness is presented in FIG. 4. The pHEMA samples had RMS of surface roughness of 31.30 nm, which was significantly (p<0.01) higher than the other polymer samples, while T:H 1:1 had the lowest roughness (0.64 nm). T:H 1:3 and pTRIS had 5.99 and 2.01 nm RMS roughness, respectively. The high roughness of pHEMA could be explained by the hydrophilicity of pHEMA and the effect of water uptake (and dehydration) on chain relaxation. Roughness tests were performed using fully dehydrated samples; the resulting shrinkage of pHEMA during the dehydration process creates a rough surface. As the other samples are hydrophobic, dehydration does not have a remarkable impact on surface roughness.

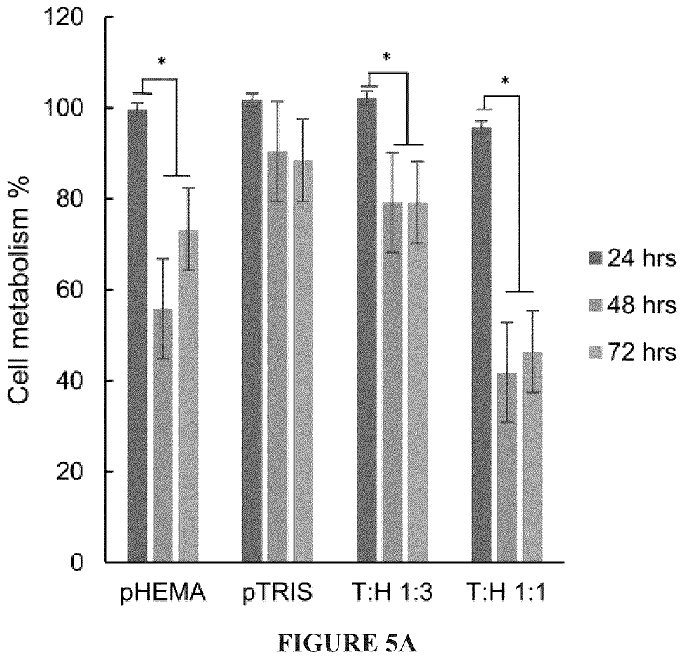

Cell metabolism and live/dead assay. As shown in FIG. 5A, the cell metabolism changed over time for all samples. After 48 h of culture, human (h)LEC metabolism decreased compared to 24 h of culture for all samples. When cell metabolic activity of each sample evaluated in 72 h, T:H 1:1 had the lowest cell metabolic activity after 48 and 72 h in comparison with cell viability of all other samples during this time. Meanwhile pTRIS had the smallest changes (reductions) in cell activity at 48 and 72 h.

Cells were stained with Calcein AM (green if living) and ethidium homodimer (red/orange if dead). As seen in FIG. 5B, nearly all cells were viable and no observable increase in the number of dead cells were noted after 24 or 48 h, indicating any decline in cellular metabolism is likely unrelated to polymer toxicity. Cytotoxicity would not be expected with these polymers as they are used in medically approved implants; decreases in metabolic activity is more likely explained by decreased cell attachment on the polymer surfaces over time. Cell detachment from the polymer substrates, particularly T:H 1:1, due to the polymers' surface physical and chemical properties could result in a progressive decline in measured metabolic activity.

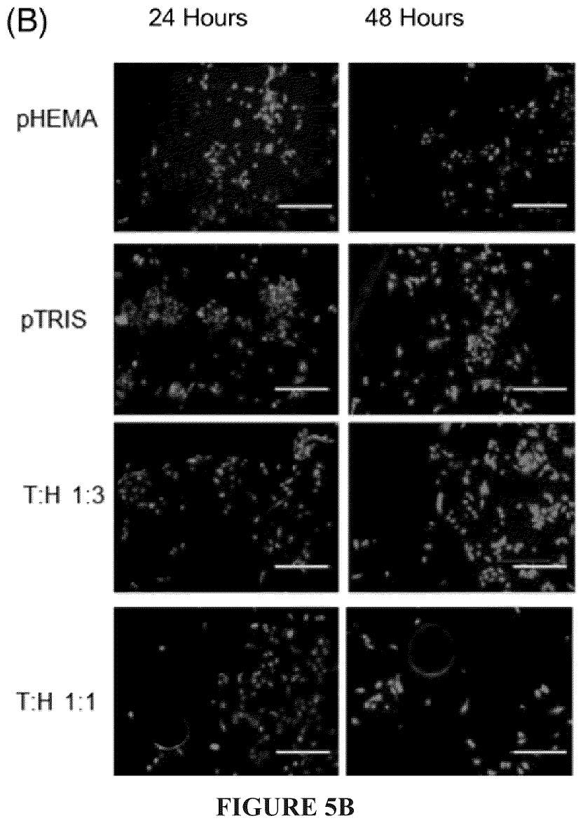

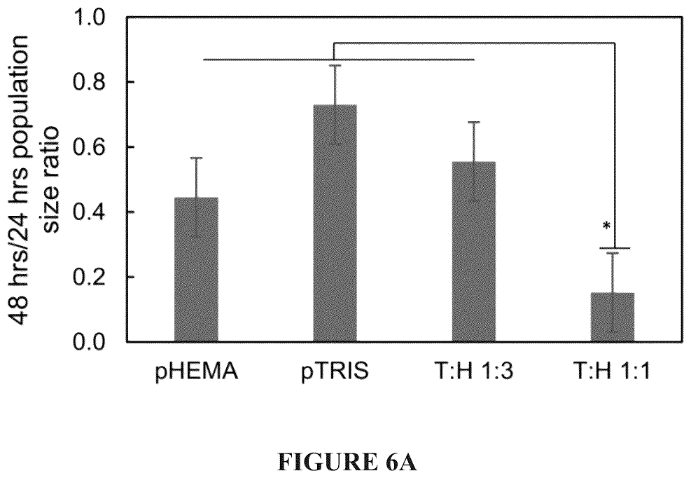

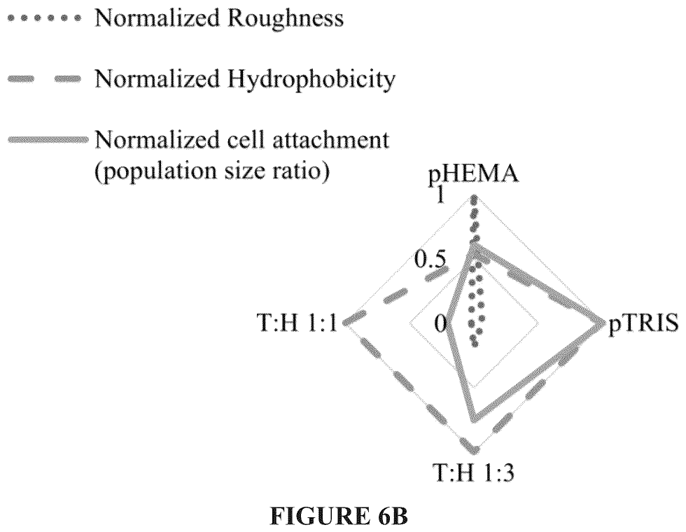

Cell attachment and cell morphology. The hLEC density was evaluated by counting the number of cells in six representative areas at 24 and 48 h and comparing the averages. As shown in FIG. 6A, among all polymer samples, T:H 1:1 had the lowest average cell density ratio (0.15), indicating that, on average, 85% of cells detached from the samples by 48 h. This phenomenon could potentially be explained by the surface smoothness of T:H 1:1. Yamakawa et al. reported that a polished surface with a lower roughness value decreased splenocyte cell attachment in vitro. Studies have also shown that cells attach to surfaces with roughness close to that of the native tissue. The human lens capsule has 10-30 nm RMS roughness, which is close to the roughness of the pHEMA (31.3 nm) and pTRIS (5.99 nm) substrates used in this study. As shown in FIG. 4, T:H 1:1 had very smooth surface in comparison with other surfaces. pHEMA had the roughest surface among all samples, and although its roughness is close to the native tissue, the cell attachment, as measured by its cell density ratio, was not the maximum. As such, it is possible that other factors known to influence cell attachment, such as surface wettability, are modulating cell behavior. Although the surface roughness may have promoted hLEC adhesion to pHEMA, the hydrophilicity of pHEMA may have deterred continued hLEC attachment over time. This suggests that both surface roughness and surface wettability are important in determining cell adhesion. In further support of this hypothesis, the pTRIS substrates had the highest cell attachment and retention among all samples. The pTRIS substrate had a surface roughness close to the lens capsule (5.99 nm) which may provide higher initial cell attachment, and also had a hydrophobic surface that enabled it to retain more hLECs on the surface. The effect of roughness and hydrophobicity on cell attachment are presented in FIG. 6B.

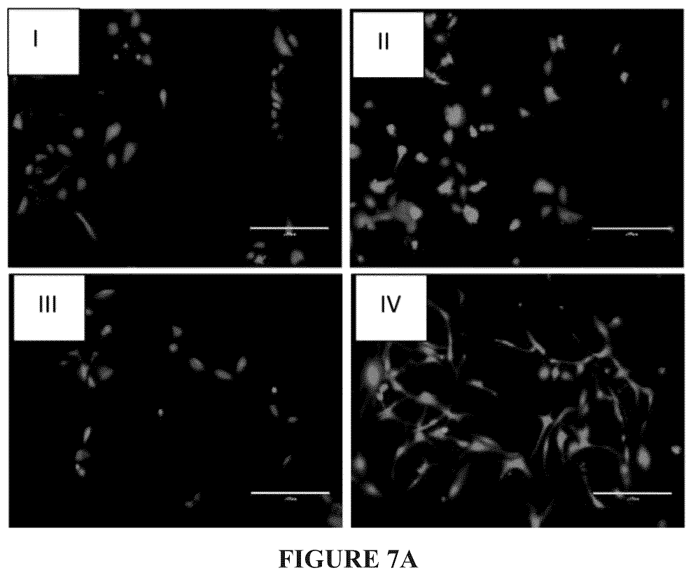

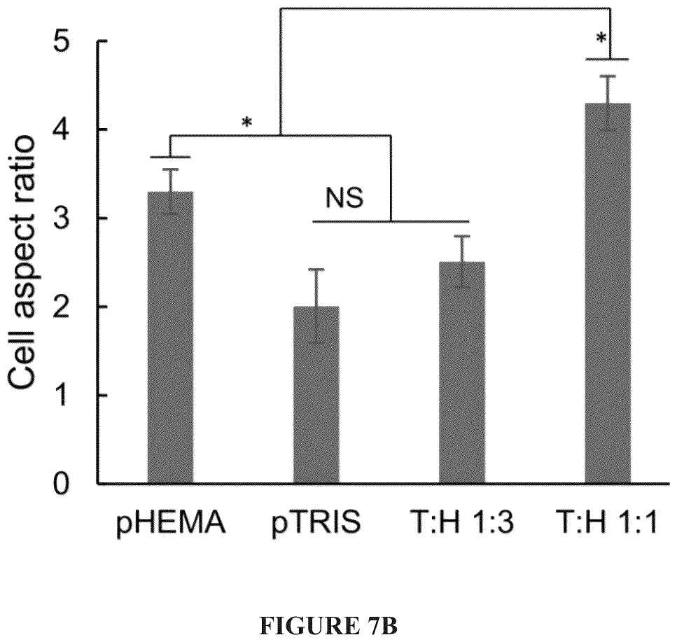

Representative images and analysis of cell morphology using aspect ratio (AR) are presented in FIGS. 7A-7B. AR is the ratio between the largest to the smallest axis of the cell. AR represents whether cells are elongated and more spindle in shape, which would be an indicator of EMT, or more circular in shape. As shown in FIG. 7B, cellular morphology differed between polymers, with cells cultured on T:H 1:1 being more elongated. This could be due to the stiffness of T:H 1:1, as hLECs may spread more on stiffer surfaces, a behavior which has been demonstrated in other cell types. This change in cell phenotype could be an indication of EMT, which is a known phenotypic change associated with PCO. The pHEMA samples had lower stiffness than T:H 1:1, and its AR was also significantly lower than T:H 1:1 (p<0.01). Both pTRIS and T:H 1:3 had the lowest AR (p<0.01), as their stiffnesses were also lower than pHEMA and T:H 1:1. The difference in AR of pTRIS and T:H 1:3 samples were not significant.

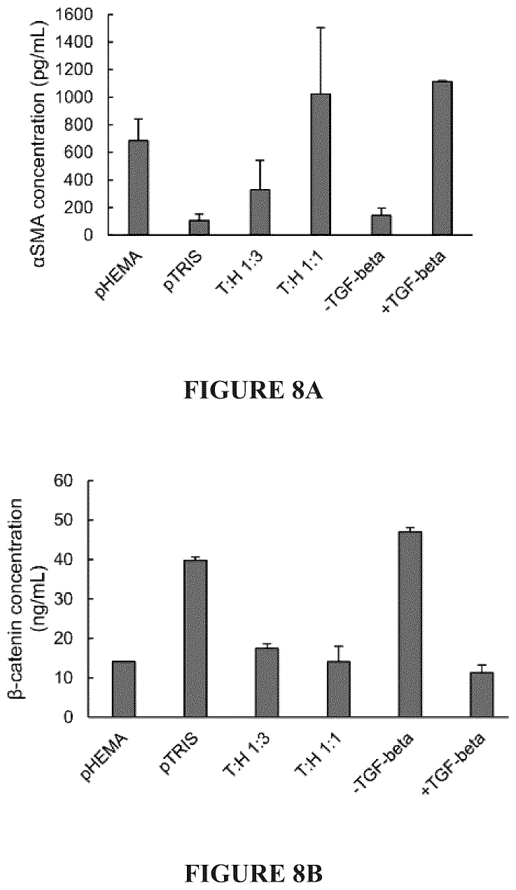

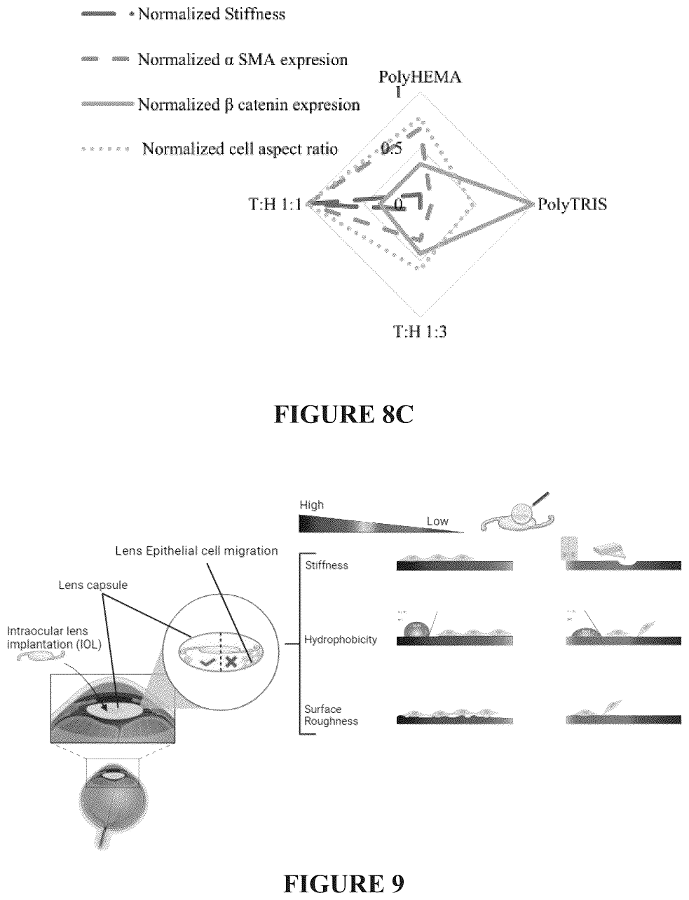

ELISA. ELISA results for α-SMA and β-Catenin expression are presented in FIGS. 8A-8C. T:H 1:1 had the highest α-SMA expression (FIG. 8A) which could be an indication of EMT. This result is in good agreement with the cell morphology assay results (FIG. 7A-7B) that showed cells on T:H 1:1 surfaces elongated more than other polymers, which is a phenotypic indication of EMT. The α-SMA expression from cells incubated on T:H 1:1 was as high as the TGF-β positive control, a known trigger for EMT. The pTRIS had the lowest α-SMA expression, indicating this polymer did not induce protein expression changes associated with EMT. This is also in agreement with the changes in cell morphology that showed that cells incubated on pTRIS had the lowest AR. EMT includes rearrangement of proteins involved in intercellular junctions and of the cadherin-catenin anchorage to the cytoskeleton. In addition to α-SMA expression, downregulation of β-catenin could be an indication of junctional protein rearrangement and consequently EMT. As shown in FIG. 8B, T:H 1:1 had lower β-catenin expression, similar to the TGF-β positive control, further supporting that this polymer induced EMT. pTRIS had the highest β-catenin expression indicating maintenance of a more epithelioid morphology that was not undergoing EMT; this was supported by lower α-SMA expression for this polymer (FIG. 8A). As shown in FIG. 8C, there is a strong connection between EMT and polymer stiffness. Normalized α-SMA expression (α-SMA concentration of each sample/maximum concentration of α-SMA among all samples) is maximal in samples with very high stiffness (T:H 1:1), and β-catenin has the lowest expression. This pattern could be seen in a sample with very low stiffness (pTRIS) where its α-SMA expression is lowest. Cell morphology changes (cell aspect ratio) with respect to the change in polymer stiffness also follow this pattern.

### Conclusion

This work has the potential to inform the design of IOLs based on amphiphilic copolymers. We prepared several copolymers and homopolymers with hydrophobic and hydrophilic monomers used in IOLs. Our results demonstrate that polymer stiffness and surface chemistry have significant effects on hLEC behavior in vitro. Based on our data, we postulate that the roughness of the pHEMA substrates provided good initial cell attachment; however, due to the hydrophilicity of the surface, cell attachment declined dramatically by 48 h. In comparison, T:H 1:1, a more hydrophobic polymer with the greatest stiffness and low roughness, had the lowest initial cell attachment and the greatest detachment rate with extended culture times. Unfortunately, cells attached to TH 1:1 were more likely to undergo EMT, which may have important implications on PCO formation. It was observed that the phenotype of hLECs was affected by the chemical and mechanical properties of the polymers, where cells grown on stiffer polymers (T:H 1:1 and pHEMA), had morphologic changes expected of EMT. When compared to T:H 1:1, pHEMA had lower α-SMA expression and its cell attachment was also lower, possibly representing a good balance between low cell attachment and less EMT. Collectively our results indicate that hLECs are responsive to surface chemistry, substrate stiffness, and surface roughness, providing potential design criteria for future IOLs that have low cell attachment and prevent EMT without the use of therapeutics. There are some limitations in this study that could be addressed in future research. As shown in this study, the mechanical properties of the polymers played an important role on cell response; similarly viscoelasticity of the polymers may also have impact on hLECs response. Surface roughness was the other factor evaluated in this Example, and these effects will be investigated further in future research.

### REFERENCES

[1] W. Halfter, S. Moes, K. Halfter, M. S. Schoenenberger, C. A. Monnier, J. Kalita, D. Asgeirsson, T. Binggeli, P. Jenoe, H. P. N. Scholl, P. B. Henrich, Exp. Eye Res. 2020, 201, 108326.
[2] M. H. Shihan, S. G. Novo, M. K. Duncan, Curr. Med. Res. Opin. 2019, 35, 1971.
[3] T. Rossi, M. R. Romano, D. Iannetta, V. Romano, L. Gualdi, I. D'Agostino, G. Ripandelli, BMJ Open Ophthalmol 2021, 6, e000464.
[4] M. Batur, A. Giil, E. Seven, E. Can, T. Ya,sar, Turk. J. Ophthalmol. 2016, 46, 205.
[5] W. F. Astle, O. Alewenah, A. D. Ingram, A. Paszuk, J. Cataract Refractive Surg. 2009, 35, 1216.
[6] P. Sen, M. Kshetrapal, C. Shah, A. Mohan, E. Jain, A. Sen, J. Cataract Refractive Surg. 2019, 45, 1380.
[7] Z. Wei, P. Gordon, C. Hao, J. Huangfu, E. Fan, X. Zhang, H. Yan, X. Fan, Cells 2022, 11, 2001.
[8] S. G. K. Gadde, A. Sridharan, N. G. Reddy, C. Jayadev, BMJ Case Rep. 2022, 15, e249264.
[9] R. M. Sheard, S. F. Goodburn, M. B. Comer, J. D. Scott, M. P. Snead, J. Cataract Refractive Surg. 2003, 29, 930.
[10] A. Grzybowski, P. Kanclerz, Asia Pac. J. Ophthalmol. 2018, 7, 339.
[11] G. Hawlina, B. Drnov ̌ sek-Olup, J. Mo ̌ zina, P. Gregorcic, Appl. Phys. A Mater. Sci. Process. 2016, 122, 118.
[12] A. R. Vasavada, M. R. Praveen, M.-J. Tassignon, S. K. Shah, V. A. Vasavada, V. A. Vasavada, J. Van Looveren, I. DeVeuster, R. H. Trivedi, J. Cataract Refractive Surg. 2011, 37, 173.
[13] Y. Zhang, C. Zhang, S. Chen, J. Hu, L. Shen, Y. Yu, Pharmaceutics 2022, 14, 1343.
[14] P. Savagner, Curr. Top. Dev. Biol. 2015, 112, 273.
[15] L. E. Scott, S. H. Weinberg, C. A. Lemmon, Front. Cell Dev. Biol. 2019, 7, 135.
[16] J. L. Leight, M. A. Wozniak, S. Chen, M. L. Lynch, C. S. Chen, Mol. Biol. Cell 2012, 23, 781.
[17] K. E. Swindle-Reilly, S. A. Carus, K. M. Schroeder, L. M. Shingler, N. K. Tram, H. L. Chandler, Invest. Ophthalmol. Vis. Sci. 2017, 58, 3193.
[18] T. Tanaka, M. Shigeta, N. Yamakawa, M. Usui, J. Cataract Refractive Surg. 2005, 31, 1648.
[19] S. Cai, C. Wu, W. Yang, W. Liang, H. Yu, L. Liu, Nanotechnol. Rev. 2020, 9, 971.
[20] D. Khang, J. Lu, C. Yao, K. M. Haberstroh, T. J. Webster, Bio-materials 2008, 29, 970.
[21] J. Hao, Y. Li, B. Li, X. Wang, H. Li, S. Liu, C. Liang, H. Wang, Appl. Biochem. Biotechnol. 2017, 183, 280.
[22] P. G. Ursell, M. Dhariwal, D. O'Boyle, J. Khan, A. Venerus, Eye 2020, 34, 960.
[23] B. P. Danysh, M. K. Duncan, Exp. Eye Res. 2009, 88, 151.
[24] L. Sutherland, C. G. Soares, Compos. Struct. 2005, 70, 287.
[25] M. Ilavska, J. Cerna, B. Bocek, J. Hrouz, Int. J. Polym. Mater. 1979, 7, 93.
[26] H. Huang, J. Y. Chung, A. J. Nolte, C. M. Stafford, Chem. Mater. 2007, 19, 6555.
[27] N. Ibaraki, L.-R. Lin, V. N. Reddy, Exp. Eye Res. 1996, 63, 683.
[28] E. Pouget, J. Tonnar, P. Lucas, P. Lacroix-Desmazes, F. Ganachaud, B. Boutevin, Chemical Reviews 2010, 110, 1233.
[29] T. Wang, S. S. Nanda, G. C. Papaefthymiou, D. K. Yi, Chem-BioChem 2020, 21, 1254.
[30] M. Vacalebre, R. Frison, C. Corsaro, F. Neri, A. Santoro, S. Conoci, E. Anastasi, M. C. Curatolo, E. Fazio, Polymers 2023, 15, 1590.
[31] A. Engler, L. Bacakova, C. Newman, A. Hategan, M. Griffin, D. Discher, Biophys. J. 2004, 86, 617.
[32] N. Yamakawa, T. Tanaka, M. Shigeta, M. Hamano, M. Usui, J. Cataract Refractive Surg. 2003, 29, 367.
[33] S. Talu, V. M. Sueiras, V. T. Moy, N. M. Ziebarth, Mol. Vis. 2018, 24, 902.
[34] T. Yeung, P. C. Georges, L. A. Flanagan, B. Marg, M. Ortiz, M. Funaki, N. Zahir, W. Ming, V. Weaver, P. A. Janmey, Cell Motil. Cytoskeleton 2005, 60, 24.
[35] L. Taliana, M. Evans, S. Ang, J. W. McAvoy, Mol. Vis. 2006, 12, 1233.
[36] J.-Z. Shao, Y. Qi, S.-S. Du, W.-W. Du, F.-Z. Li, F.-Y. Zhang, Int. J. Ophthalmol. 2018, 11, 1253.
[37] Y. Zhang, W. Huang, Med. Sci. Monit. 2018, 24, 7424.
[38] S. Basu, S. Cheriyamundath, A. Ben-Ze'ev, F1000Research 2018, 7, 7.
[39] S. Krag, T. T. Andreassen, Prog. Retin. Eye Res. 2003, 22, 749.
[39] A. Kozbial, Z. Li, C. Conaway, R. McGinley, S. Dhingra, V. Vahdat, F. Zhou, B. D'Urso,
H. Liu, L. Li, Langmuir 2014, 30, 8598.

### Example 2. The Effect of Viscoelasticity and Surface Topography Modification on Human Lens Epithelial Cells—a Potential Approach to Prevent Posterior Capsule Opacification

### Introduction

Prevention of posterior capsule opacification remains an unsolved problem after cataract surgery. Many factors could interfere with human lens epithelial cell (hLEC) response to cataract surgery and intraocular lens (IOL) implantation. Epithelial-to-mesenchymal transition (EMT) is known as the primary mechanism for PCO incidence. Growth factors released after cataract surgery and their effect on the cellular response after the procedure have been widely studied. In addition to chemical factors that induce a cellular response, IOL materials could potentially interfere with cellular behavior and cause EMT. One study on LEC response to the viscoelastic device (VOD) showed morphological changes, and LEC removed using a VOD were generally thinner, and their nuclei were compressed. The other study shows different IOL designs have different incidences of PCO, showing that hLECs may respond differently to the IOLs composed of different polymers—or some other material properties. Several studies show cells respond to the changes in the mechanical properties of their microenvironment. Most of the IOLs in clinical use are made of polymers with mechanical properties very different (Young's modulus of IOLs >0.5 MPa) from the native tissue (Young's modulus of human lens ˜1 kPa) which they replace.

We hypothesize that this mismatch in viscoelastic properties may be a factor contributing to PCO after IOL implantation. Therefore, hLEC response to viscoelastic polymers was studied. We relied on crosslinking the polymer chains to modulate viscoelastic properties. There are different techniques for polymer crosslinking, including chemical crosslinking (e.g., using chemical agents) and physical crosslinking (e.g., thermal transitions). The freeze-thaw (F-T) technique is a safe crosslinking technique to make polymers with different mechanical properties by controlling freezing times and the number of cycles. Polyvinyl alcohol (PVA) was selected for this study due to its demonstrated lack of toxicity and use in ophthalmic applications. Cell types also exhibit different behavior in response to the surface topography. We therefore hypothesize that surface topography may be another factor that can influence LEC response to an implant. Fraser et al. showed corneal epithelial cells aligned on nanoscale and microscale features. Respond to surface topography is dependent on cell type. For example, bone-derived cells respond to the size of the patterns differently. On submicron roughness, their thickness increases, while on hexagonal arrays of hemispherical smooth cavities of 30 μm and 100 μm diameter, cells preferentially attach inside the cavities and in 30 μm feature size adopt a three-dimensional shape. Their morphology changes in 100 μm cavities and turns flat and more spread out. Our previous study showed that LECs responded to the surface roughness, and their attachment potentially increased with roughness. Therefore, the objective of this study was to evaluate LEC response to viscoelasticity and surface topography to evaluate two potential ways to mitigate fibrotic complications after cataract surgery.

### Materials and Methods

Polyvinyl alcohol (PVA, MW 85k-124K, +99% hydrolyzed) (Sigma-Aldrich, US), Dimethyl Sulfoxide (DMSO, Sigma-Aldrich, US), Polymethyl methacrylate (PMMA) (Sigma-Aldrich, US), 1,1,1,3,3,3-hexafluoro-2-propanol (HFP) (Oakwood Products Inc, Estill, SC), Anisole (Sigma-Aldrich, France), Polycaprolactone (PCL) (Sigma-Aldrich, UK), Dulbecco's Modified Eagle Medium (DMEM, Gibco, US), fetal bovine serum (FBS, Gibco, US), phosphate-buffered saline (PBS, Sigma Life Science, UK), penicillin-streptomycin 100× (Corning, VA, US), Live/dead assay kit (Invitrogen Carlsbad, CA, US), dialysis membrane (MWCO 6-8K) (Spectra/Pro, CA, US), human α-SMA ELISA kit (abeam, ab240678), human beta catenin ELISA kit (abeam, ab275100), Western blot kit (Bio Rad, CA, US).

Viscoelastic polymer preparation. Table 3 shows formulations prepared. PVA was dissolved in 80:20 v/v of DMSO: deionized water (DI H₂O) at 80-90° C. The solution was mixed for 2 hours for a uniform and transparent solution. 500 μl of solution was transferred into each well of a 24-well plate. Then, samples were kept in a freezer at −20° C. for a proper time (freezing cycle). Then, they were removed and kept at room temperature (thaw cycle). The F-T cycle was repeated as needed. After the F-T cycle, samples were transferred to a dialysis bag and kept in 1000 ml (DI water) for three days to replace DMSO with DI water. Different samples with different PVA concentrations (10 w/v % and 20 w/v %) prepared. Based on rheological studies, three samples were selected for further study.

TABLE 3
Samples prepared for the viscoelasticity study

| | PVA | | |
|---|---|---|---|
| Sample name | concentration | F-T Time (hr.) | Number of cycles |
| PVA 10, 0.25, 1 | 10% | 0.25-2 | 1 |
| PVA10, 2, 1 | 10% | 2-2 | 1 |
| PVA 10, 24, 3 | 10% | 24-2 | 3 |
| PVA 10, 24, 1 | 10% | 24-2 | 1 |
| PVA 20, 24, 3 | 20% | 24-2 | 3 |

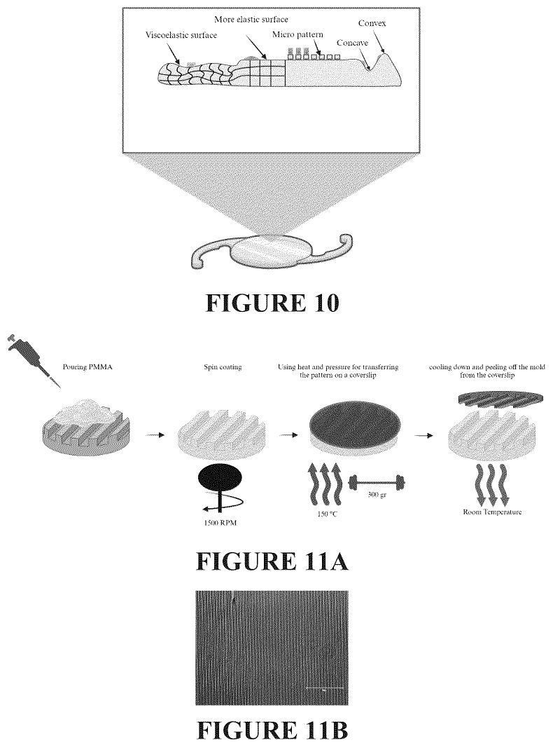

Micropatterning. The Imprinting technique is used for transferring patterns from mold to the substrate. Briefly, the mold is coated with Polymethyl methacrylate (PMMA) (11% in anisole) using a spin coater at 1500 rpm; then the mold is pressed onto a glass coverslip (10 mm diameter) for 10 minutes at 150° C. Finally, the coverslip is cooled to room temperature. After five minutes, the mold is peeled off for further evaluation. This process is illustrated in FIG. 11.

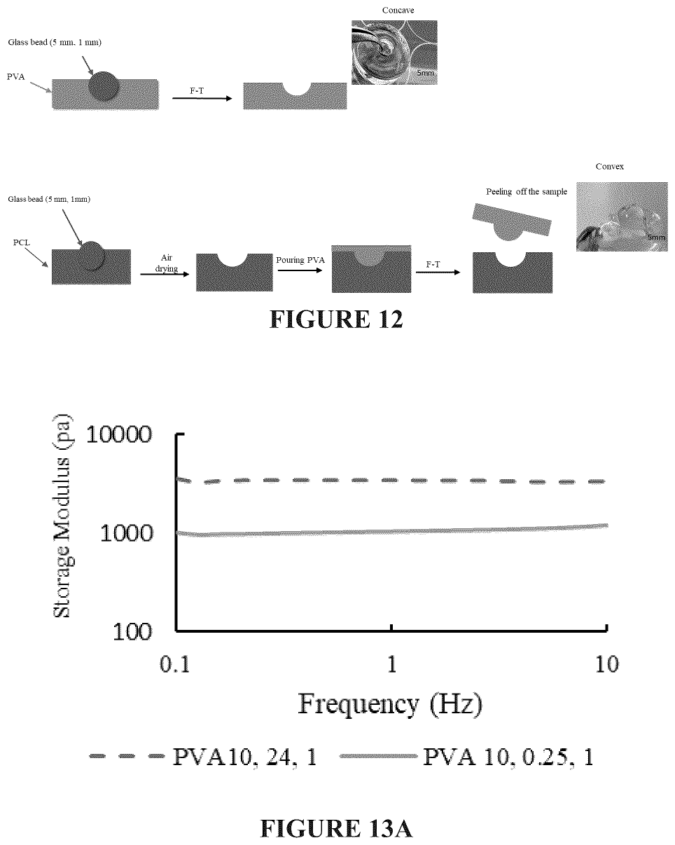

Convex and concave surface preparation. To make a concave surface, 1 ml of PVA (10%) was poured on a 48-well plate, a glass bead (1 mm/5 mm) was pressed in the polymer solution, and frozen at −20° C. for 2 hours and thawed at room temperature. Then, the glass bead was removed. For convex topography, we prepared a convex mold using PCL (10% in HFP), just like the PVA convex molds, but PCL was air-dried instead of using the freeze-drying process. Then, 1 ml of PVA (10%) was poured into a PCL mold and frozen/thawed (−20° C./room temperature). These methods are schematically illustrated in FIG. 12.

Rheological test to evaluate polymer viscoelasticity. Oscillatory parallel plate rheological measurements were conducted on PVA hydrogels using a Kinexus rheometer (Malvern Instruments, UK). Hydrogels were hydrated in culture medium for 24 hrs prior to testing. All samples were assessed in triplicate (using individual samples) at a temperature of 37° C. using a stainless steel parallel plate. A frequency sweep was applied from 0.1-10 Hz with 1% strain, while a constant normal force of 1 N was exerted on the samples.

Scanning electron microscopy (SEM). Cell morphology and polymer surface topography were studied using a scanning electron microscope (Apreo high-resolution SEM). For the cell morphology assay, cells were fixed by submerging samples in formaldehyde (4% in PBS) and then dehydrated in gradual alcohol (50%, 60%, 70%, 95%, and 100%) for five minutes at room temperature at each concentration, and mounted for coating. Finally, samples were coated with gold using Pelco Sputter Coater.

Cell attachment tests. For viscoelastic and concave/convex polymers, 500 μl of each sample were placed in 24 well plate (wells were entirely covered with hydrogel), and each sample was seeded with 2*10⁴/cm² hLECs. After 24 hrs., the live/dead assay kit was used for staining, and images were taken using a fluorescent microscope (Invitrogen, EVOS 5000, WA, US). ImageJ is used for image analysis.

Western Blot. For viscoelastic and concave/convex polymers, 1 ml of each sample was placed in 12 well plates (wells were entirely covered with hydrogel), and for micropatterned samples, each patterned coverslip was placed in 24 well plates. All the samples were seeded with 3*10⁴/cm² hLECs. After 24 hours, the protein was isolated, and expression of α-SMA and β-catenin were measured using commercially available Western Blot, following manufacturer instructions. Each hydrogel was assessed in triplicate-cells cultured on the fully elastic surface of the well plate served as a positive control.

Enzyme-linked immunosorbent assay (ELISA). Experiments were performed using a 12-well plate (1 ml hydrogel per well for viscoelastic and concave/convex polymers and one coverslip per well for micropattern surface). Samples were added to each well, and cells (3*10⁴ cells/cm²) were seeded into the center of each well and cultured for 24 hours. Protein was isolated and quantified. Expression of α-SMA and β-catenin were measured in protein lysate using commercially available ELISAs, following manufacturer instructions. Each sample was tested in duplicate. Two wells with thin layers of PVA were seeded as a control for the viscoelastic polymer sample. A coverslip coated with PMMA without any pattern was seeded as a control for micropattern samples.

Quantitative real-time polymerase chain reaction PCR. Experiments were performed using a 12-well plate (1 ml hydrogel for viscoelastic and concave/convex polymers and one coverslip for micropatterned surface). Samples were added to each well, and cells (3*10⁴ cells/cm²) were seeded into the center of each well and cultured for 24 hours. The gene expression analysis by real-time RT-PCR was used to evaluate the expression of EMT markers: α-SMA, β-catenin, and vimentin.

### Results

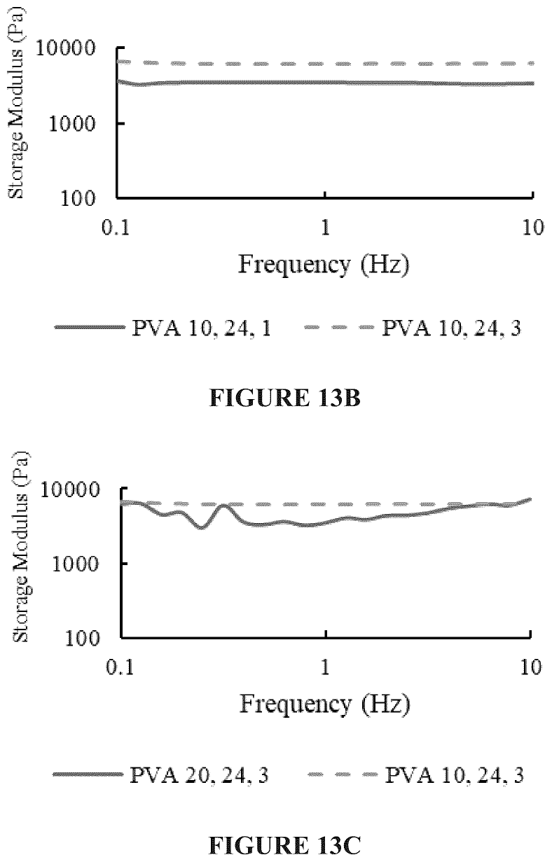

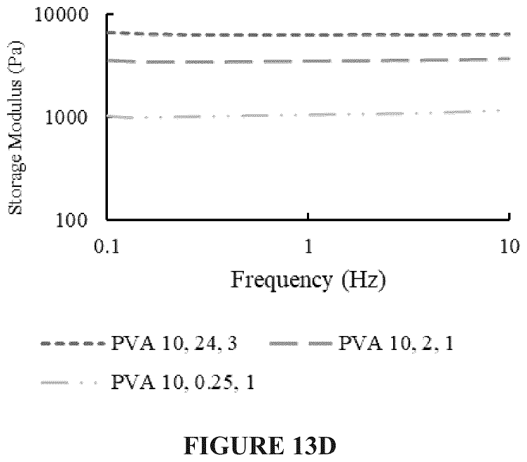

Rheological assay for hydrogel viscoelasticity evaluation. FIGS. 13A-13D present the effect of several factors on hydrogel viscoelasticity. All samples had constant thaw time. As shown in FIG. 13A, a longer freezing time with fixed thaw time (2 hours) and freezing cycle (one cycle) increases storage modulus, and PVA 10, 24, 1 had higher elastic properties than PVA 10, 0.25, 1. The number of F-T cycles is the other factor that remarkably impacted viscoelasticity (see FIG. 13B). PVA 10, 24, and 3 with three cycles of F-T had a higher storage modulus when compared to a sample with one F-T cycle at the same freezing time and concentration. As presented in FIG. 13C, the viscoelasticity of PVA is independent of concentration, and storage modulus is almost the same for 10% and 20% formulations. Three samples with various viscoelasticity are compared in FIG. 13D. These were selected for further evaluation.

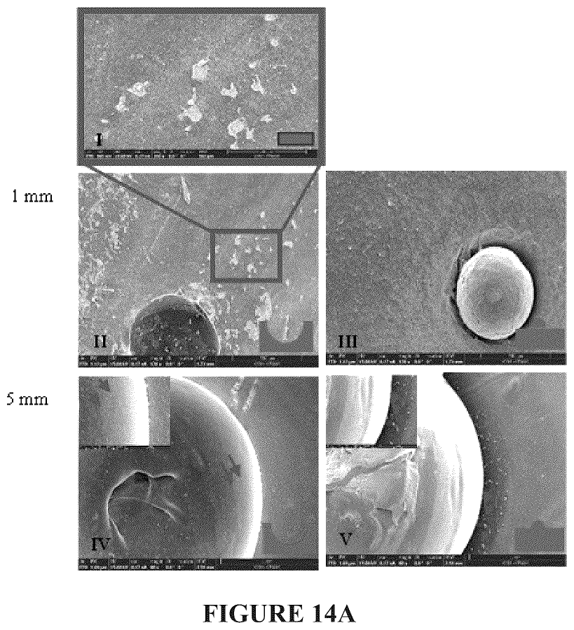

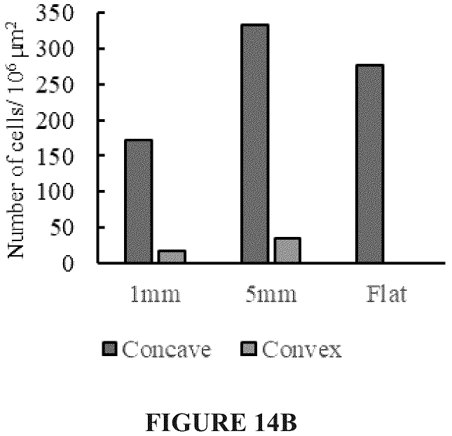

SEM analysis on cell response to the surface topography. As shown in FIGS. 14A-14B, hLECs could easily attach to the wall and the bottom of the holes, while on concave surfaces, they had more difficulty with migration and attachment to the top or wall. Studies evaluating the response of renal proximal tubule epithelial cells (RPTECs) to concave and convex topography have demonstrated that renal cells preferentially attached to concave curvature rather than convex, which is similar to the results obtained using human lens epithelial cells. Our preliminary results also indicated that cell attachment on a flat surface did not differ significantly from that on a concave surface.

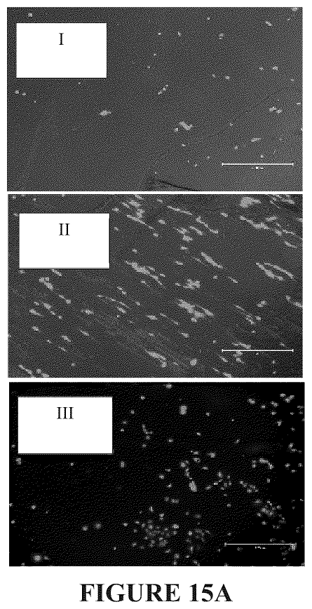

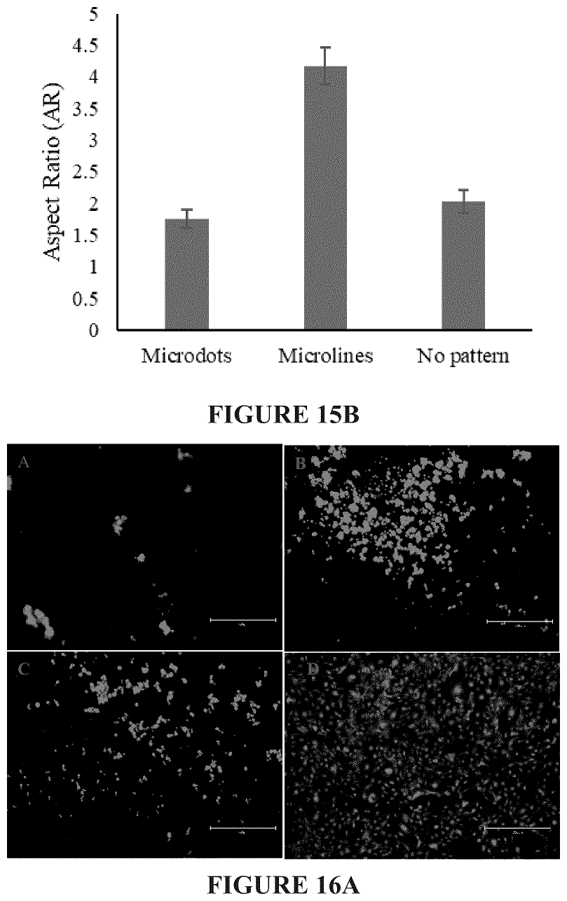

Cell morphology changes in response to micropatterns (microdots and microlines). The shape, distance and orientation of micropatterns define cell-substrate attachment and cell spreading. As a cell spreads, it attaches on the substrate and develops larger focal adhesions and forms actin filaments which can pull on the substrate. Directional patterning (e.g., microlines) promotes actin expression in specific direction (toward the pattern). As shown in FIGS. 15A-15B, hLEC on microline patterns tend to spread in the direction of the lines and their aspect ratios are significantly (p<0.05) higher than microdot and unpatterned (control) samples. Interestingly, hLECs on microdot patterns had slightly lower aspect ratios compared to unpatterned sample. This could be explained by the shape of the dot pattens evaluated: cells feel and touch it and spread in circular shape, while on unpatterned samples of the same material cells might spread in different directions randomly. These data indicate an ability to influence hLEC adhesion and aspect ratios, which could potentially influence EMT.

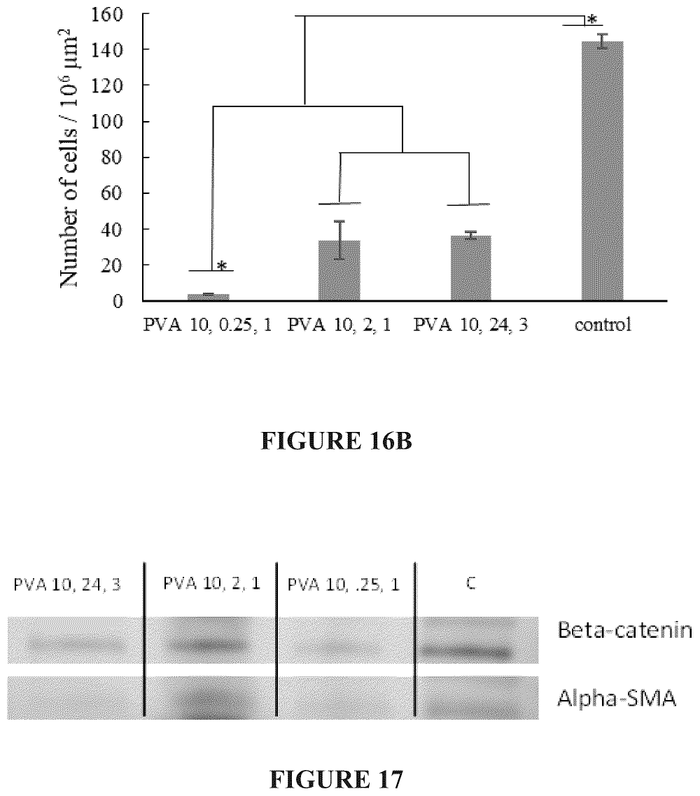

Cell attachment to viscoelastic polymers. Cell adhesion to a surface is multifunctional; several different factors could interfere with the cell's response to the surface. The surface's mechanical properties are critical factors that play a vital role in cell response. FIG. 16A (Panels A-D) shows that hLECs tend to attach on more elastic than viscous surfaces (e.g., surfaces with less dampening effects). PVA 10, 0.25, 1 sample (FIG. 16A, Panel A) with the lowest elasticity had the lowest number of attached hLECs, while PVA 10, 2, 1 (FIG. 16A, Panel B) and PVA 10, 24, 3 FIG. 16A, Panel C with higher moduli of elasticity have significantly higher cell attachment when compared to PVA 10, 0.25, 1 (p<0.05). Cell attachment on PVA 10, 2, 1 and PVA 10, 24, 3 was not significantly different; meanwhile, the control sample (cells on a fully elastic polymer well plate) had considerably higher cell attachment (p<0.05) than all viscoelastic samples evaluated. It appears there is a threshold for the cell response to the polymer viscoelasticity. It might be connected to the viscoelasticity of the human lens epithelial cells themselves or the human lens as a whole (anterior, posterior, cortex, and nucleus sections) that has a storage modulus (G′) in the range of 300-4000 Pa, which depends on the age and section of the lens.

Western Blot to investigate the effect of viscoelasticity on hLEC. Alpha-SMA and beta-catenin were selected as two markers for epithelial-mesenchymal transition (EMT), and a western blot assay was performed (FIG. 17). PVA 10, 2, 1 had higher alpha-SMA expression, which is a marker for EMT, than PVA 10, 0.25, 1 and PVA 10, 24, 3. More elastic polymers appear to promote alpha-SMA expression (PVA 10, 2, 1 vs. PVA 10, 0.25, 1). Surprisingly, cells on PVA 10, 24, 3, which had the highest elasticity, did not appear to follow this trend. These studies are currently being repeated to validate these findings.

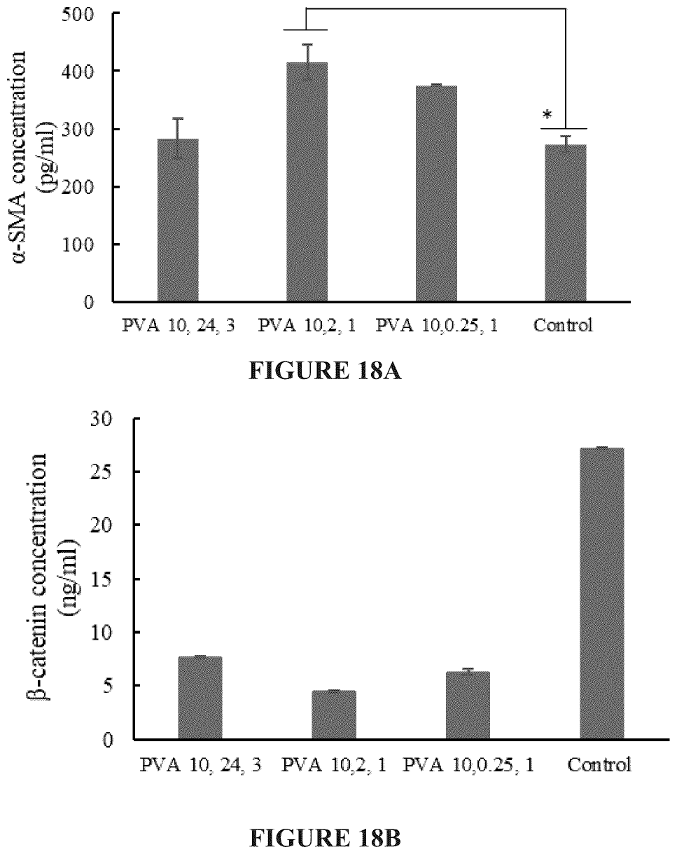

ELISA assay to investigate the effect of viscoelasticity on hLEC. As shown in FIGS. 18A-18B, expression of both Alpha-SMA and beta-catenin was influenced by substrate viscoelasticity. Higher concentration of α-SMA could be an indication of EMT, which was seen for the PVA 10,2, 1 sample. This was significant compared to the control. All viscoelastic surfaces had significantly lower beta-catenin expression than the tissue culture plate control, which is purely elastic.

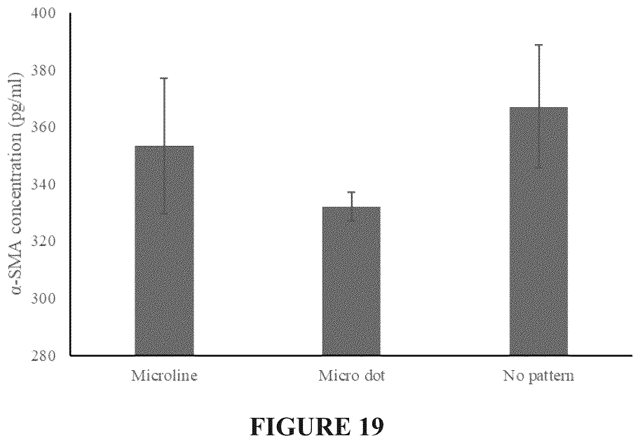

ELISA assay to investigate the effect of micropatterns on hLEC. As shown in FIG. 19, α-SMA expression from cells seeded on microlines was higher than on microdot patterns. Higher concentration of α-SMA could be an indication of EMT. The noted changes in cell morphology were also in good agreement with these ELISA results. As presented in FIG. 15B, cells on microline patterns were more elongated and had higher aspect ratios that can be indicative of EMT. However, the concentration of α-SMA was not significantly higher than the unpatterned (control) sample. On the other hand, microdot patterning had the lowest signaling of α-SMA.

### Conclusion

While studies are ongoing, particularly quantitative biological marker expression, preliminary results indicate that hLECs respond not only to substrate chemistry and stiffness, but also substrate viscoelasticity and surface topography. Viscoelasticity, the combination of viscous forces with elastic forces (or viscosity with elastic moduli), has remained understudied in cell response, particularly in the eye. Modulating IOL viscoelasticity may be one potential approach to mitigating hLEC response and preventing development of PCO. As injectable gels or accommodating lens refills undergo further research and development, viscoelasticity will be an important consideration. Further, our preliminary results indicate that micropatterning the edges of an IOL may enable control over residual hLEC attachment, potentially preventing migration to the visual axis of the IOL.

### REFERENCES

[1] Saika, S., et al., TGFβ-Smad signalling in postoperative human lens epithelial cells. British journal of ophthalmology, 2002. 86(12): p. 1428-1433.
[2] Meacock, W., D. Spalton, and M. Stanford, Role of cytokines in the pathogenesis of posterior capsule opacification. British journal of ophthalmology, 2000. 84(3): p. 332-336.
[3] Hales, A. M., C. G. Chamberlain, and J. W. McAvoy, Susceptibility to TGFβ2-induced cataract increases with aging in the rat. Investigative ophthalmology & visual science, 2000. 41(11): p. 3544-3551.
[4] Budo, C., et al., Effect of ophthalmic viscosurgical devices on lens epithelial cells: a morphological study. Journal of Cataract & Refractive Surgery, 2003. 29(12): p. 2411-2418.
[5] Ursell, P. G., et al., 5 year incidence of YAG capsulotomy and PCO after cataract surgery with single-piece monofocal intraocular lenses: a real-world evidence study of 20,763 eyes. Eye, 2020. 34(5): p. 960-968.
[6] Ghosh, K., et al., Cell adaptation to a physiologically relevant ECM mimic with different viscoelastic properties. Biomaterials, 2007. 28(4): p. 671-679.
[7] Gupta, S., T. J. Webster, and A. Sinha, Evolution of PVA gels prepared without crosslinking agents as a cell adhesive surface. Journal of Materials Science: Materials in Medicine, 2011. 22: p. 1763-1772.
[8] Xu, J., et al., Carbazole-grafted silicone hydrogel with a high refractive index for intraocular lens. RSC advances, 2015. 5(89): p. 72736-72744.
[9] Riehle, N., et al., Influence of PDMS molecular weight on transparency and mechanical properties of soft polysiloxane-urea-elastomers for intraocular lens application. European polymer journal, 2018. 101: p. 190-201.
[10] de Groot, J. H., et al., Hydrogels for an accommodating intraocular lens. An explorative study. Biomacromolecules, 2003. 4(3): p. 608-616.
[11] Shiblee, M. N. I., et al., 3D printing of shape memory hydrogels with tunable mechanical properties. Soft matter, 2018. 14(38): p. 7809-7817.
[12] Fisher, R., The elastic constants of the human lens. The Journal of physiology, 1971. 212(1): p. 147-180.
[13] Karayilan, M., L. Clamen, and M. L. Becker, Polymeric materials for eye surface and intraocular applications. Biomacromolecules, 2021. 22(2): p. 223-261.
[14] Moffat, K. L. and K. G. Marra, Biodegradable poly (ethylene glycol) hydrogels crosslinked with genipin for tissue engineering applications. Journal of Biomedical Materials Research Part B: Applied Biomaterials: An Official Journal of The Society for Biomaterials, The Japanese Society for Biomaterials, and The Australian Society for Biomaterials and the Korean Society for Biomaterials, 2004. 71(1): p. 181-187.
[15] Rudra, R., V. Kumar, and P. P. Kundu, Acid catalysed cross-linking of poly vinyl alcohol (PVA) by glutaraldehyde: effect of crosslink density on the characteristics of PVA membranes used in single chambered microbialfuel cells. RSC advances, 2015. 5(101): p. 83436-83447.
[16] Miranda, T. M. R., A. R. Gongalves, and M. T. P. Amorim, Ultraviolet-induced crosslinking of poly (vinyl alcohol) evaluated by principal component analysis of FTIR spectra. Polymer international, 2001. 50(10): p. 1068-1072.
[17] Hou, Y., et al., Preparation of PVA hydrogel with high-transparence and investigations of its transparent mechanism. RSC Advances, 2015. 5(31): p. 24023-24030.
[18] Hassan, C. M. and N. A. Peppas, Structure and morphology of freeze/thawed PVA hydrogels. Macromolecules, 2000. 33(7): p. 2472-2479.
[19] Fukumori, T. and T. Nakaoki, Significant improvement of mechanical properties for polyvinyl alcohol film prepared from freeze/thaw cycled gel. Open Journal of Organic Polymer Materials, 2013. 2013.
[20] Gupta, S., S. Goswami, and A. Sinha, A combined effect of freeze-thaw cycles and polymer concentration on the structure and mechanical properties of transparent PVA gels. Biomedical Materials, 2012. 7(1): p. 015006.
[21] Zha, X.-J., et al., Nanofibrillar poly (vinyl alcohol) ionic organohydrogels for smart contact lens and human-interactive sensing. ACS applied materials & interfaces, 2020. 12(20): p. 23514-23522.
[22] Kharaghani, D., et al., Active loading graphite/hydroxyapatite into the stable hydroxyethyl cellulose scaffold nanofibers for artificial cornea application. Cellulose, 2020. 27: p. 3319-3334.
[23] Hyon, S.-H., et al., Poly (vinyl alcohol) hydrogels as soft contact lens material. Journal of Biomaterials Science, Polymer Edition, 1994. 5(5): p. 397-406.
[24] Tummala, G. K., R. Rojas, and A. Mihranyan, Poly (vinyl alcohol) hydrogels reinforced with nanocellulose for ophthalmic applications: general characteristics and optical properties. The Journal of Physical Chemistry B, 2016. 120(51): p. 13094-13101.
[25] Anselme, K. and M. Bigerelle, Role of materials surface topography on mammalian cell response. International Materials Reviews, 2011. 56(4): p. 243-266.
[26] Fraser, S. A., et al., Sub-micron and nanoscale feature depth modulates alignment of stromal fibroblasts and corneal epithelial cells in serum-rich and serum-free media. Journal of biomedical materials research Part A, 2008. 86(3): p. 725-735.
[27] Zinger, O., et al., Time-dependent morphology and adhesion of osteoblastic cells on titanium model surfaces featuring scale-resolved topography. Biomaterials, 2004. 25(14): p. 2695-2711.
[28] van Gaal, R. C., et al., Renal epithelial cell responses to supramolecular thermoplastic elastomeric concave and convex structures. Advanced Materials Interfaces, 2021. 8(1): p. 2001490.
[29] Théry, M., Micropatterning as a tool to decipher cell morphogenesis and functions. Journal of cell science, 2010. 123(24): p. 4201-4213.
[30] Zareidoost, A., et al., The relationship of surface roughness and cell response of chemical surface modification of titanium. Journal of Materials Science: Materials in Medicine, 2012. 23: p. 1479-1488.
[31] Mandal, K., et al., Opposite responses of normal hepatocytes and hepatocellular carcinoma cells to substrate viscoelasticity. Biomaterials science, 2020. 8(5): p. 1316-1328.
[32] Engler, A. J., et al., Matrix elasticity directs stem cell lineage specification. Cell, 2006. 126(4): p. 677-689.
[33] Krag, S. and T. T. Andreassen, Mechanical properties of the human lens capsule. Progress in retinal and eye research, 2003. 22(6): p. 749-767.

The materials, devices, and methods of the appended claims are not limited in scope by the specific materials, devices, and methods described herein, which are intended as illustrations of a few aspects of the claims. Any materials, devices, and methods that are functionally equivalent are intended to fall within the scope of the claims. Various modifications of the materials, devices, and methods in addition to those shown and described herein are intended to fall within the scope of the appended claims. Further, while only certain representative materials, devices, and methods disclosed herein are specifically described, other combinations of the materials and devices also are intended to fall within the scope of the appended claims, even if not specifically recited. Thus, a combination of steps, elements, components, or constituents may be explicitly mentioned herein or less, however, other combinations of elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Unless defined otherwise, all technical and scientific terms used herein have the same meanings as commonly understood by one of skill in the art to which the disclosed invention belongs. Publications cited herein and the materials for which they are cited are specifically incorporated by reference.

We claim:

1. A method for decreasing the rate of posterior capsular opacification (PCO) observed in a population of subjects following implantation of an intraocular lens, wherein the intraocular lens comprises an optic disposed about an optical axis comprising an anterior surface and an opposing posterior surface, the surfaces configured to focus light when implanted within a capsular bag of an eye, and a support structure coupled to the optic; the method comprising:
modifying an edge region of the optic to exhibit a surface chemistry, a mechanical property, a curvature, a micropattern, or a combination thereof that reduces the rate of lens epithelial cell (LEC) attachment, prevents LEC cells from undergoing an epithelial-mesenchymal transition (EMT), or a combination thereof following implantation in a subject in need thereof.

2. The method of claim 1, wherein modifying the edge region of the optic comprises increasing the hydrophobicity of the edge region of the optic.

3. The method of claim 2, wherein the edge region of the optic exhibits a water contact angle of from 80° to 110°.

4. The method of claim 1, wherein modifying the edge region of the optic comprises reducing the stiffness of the edge region of the optic.

5. The method of claim 4, wherein the edge region of the optic exhibits a Young's modulus of from 0.1 MPa to 6500 MPa.

6. The method of claim 1, wherein modifying the edge region of the optic comprises increasing the elasticity of the edge region of the optic.

7. The method of claim 6, wherein the edge region of the optic exhibits a storage modulus of from 10 Pa to 25,000 Pa.

8. The method of claim 1, wherein modifying the edge region of the optic comprises patterning the edge region of the optic to increase the root mean square (RMS) roughness of the edge region to from 0.1 nm to 50.0 nm.

9. The method of claim 1, wherein modifying the edge region of the optic comprises patterning the edge region of the optic to introduce a plurality of protrusions within the edge region.

10. The method of claim 9, wherein the plurality of protrusions comprise an array of microdots or micropillars.

11. The method of claim 10, wherein the microdots or micropillars have a largest average cross-sectional dimension of from 1 micron to 10 microns.

12. The method of claim 11, wherein the array of microdots or micropillars exhibits an average spacing of from 500 nm to 10 microns.

13. The method of claim 1, wherein modifying the edge region of the optic comprises modifying the curvature of the edge region of the optic.

14. The method of claim 13, wherein modifying the curvature of the edge region comprises introducing convex curvature within the edge region of the optic.

15. An intraocular lens, comprising:
an optic disposed about an optical axis comprising an anterior surface and an opposing posterior surface, the surfaces configured to focus light when implanted within a capsular bag of an eye; and
a support structure coupled to the optic;
wherein the optic comprises an edge region having a Young's modulus of from 0.1 MPa to 6500 MPa.

16. The lens of claim 15, wherein the edge region of the optic exhibits a water contact angle of from 80° to 110°.

17. The lens of claim 15, the edge region of the optic exhibits a storage modulus of from 10 Pa to 25,000 Pa.

18. The lens of claim 15, wherein the edge region exhibits a root mean square (RMS) roughness of the edge region to from 0.1 nm to 50.0 nm.

19. An intraocular lens, comprising:
an optic disposed about an optical axis comprising an anterior surface and an opposing posterior surface, the surfaces configured to focus light when implanted within a capsular bag of an eye; and
a support structure coupled to the optic;
wherein the optic comprises a micropatterned edge region comprising an array of microdots or micropillars.

20. The lens of claim 19, wherein the microdots or micropillars have a largest average cross-sectional dimension of from 1 micron to 10 microns.
